(19) United States
(12) Patent Application Publication
(10) Pub. No.: US 2014/0148454 A1
(43) Pub. Date: May 29, 2014

(54) SUBSTITUTED AMINO-ACRYLCARBOXAMIDES AS KCNQ2/3 MODULATORS

(71) Applicant: GRÜNENTHAL GMBH, Aachen (DE)

(72) Inventors: Simon LUCAS, Wolfsgraben (AT); Sven Kühnert, Duren (DE); Gregor Bahrenberg, Monschau-Konzen (DE); Wolfgang Schröder, Aachen (DE)

(73) Assignee: GRÜNENTHAL GMBH, Aachen (DE)

(21) Appl. No.: 14/091,378

(22) Filed: Nov. 27, 2013

Related U.S. Application Data
(60) Provisional application No. 61/730,532, filed on Nov. 28, 2012.

(30) Foreign Application Priority Data
Nov. 28, 2012 (EP) 12007991.8

(51) Int. Cl. A61K 31/5377; C07D 241/24; C07D 401/04; C07D 213/81; C07D 417/12; A61K 31/427; C07D 239/28

(52) U.S. Cl. CPC A61K 31/5377; A61K 31/427; C07D 241/24; C07D 239/28; C07D 213/81; C07D 417/12; C07D 401/04
USPC 514/235.8; 514/235.5; 514/370; 544/120; 544/122; 544/131; 548/194

(57) ABSTRACT

The invention relates to amino-arylcarboxamides, to pharmaceutical compositions containing these compounds and also to these compounds for use in the treatment and/or prophylaxis of pain and further diseases and/or disorders.

This application claims priority of U.S. Provisional Patent Application No. 61/730,532, filed on Nov. 28, 2012, and European Patent Application No. 12007991.8, filed on Nov. 28, 2012, the entire contents of which patent applications are incorporated herein by reference.

The invention relates to substituted amino-arylcarboxamides, to pharmaceutical compositions containing these compounds and also to these compounds for use in the treatment and/or prophylaxis of pain and further diseases and/or disorders.

The treatment of pain, in particular of neuropathic pain, is of great importance in medicine. There is a worldwide need for effective pain therapies. The urgent need for action for a target-orientated treatment of chronic and non-chronic states of pain appropriate for the patient, by which is to be understood the successful and satisfactory treatment of pain for the patient, is also documented in the large number of scientific works which have recently been published in the field of applied analgesics and of fundamental research into nociception.

A pathophysiological feature of chronic pain is the overexcitability of neurons. Neuronal excitability is influenced decisively by the activity of K⁺ channels, since these determine decisively the resting membrane potential of the cell and therefore the excitability threshold. Heteromeric K⁺ channels of the molecular subtype KCNQ2/3 (Kv7.2/7.3) are expressed in neurons of various regions of the central (hippocampus, amygdala) and peripheral (dorsal root ganglia) nervous system and regulate the excitability thereof. Activation of KCNQ2/3 K⁺ channels leads to a hyperpolarization of the cell membrane and, accompanying this, to a decrease in the electrical excitability of these neurons. KCNQ2/3-expressing neurons of the dorsal root ganglia are involved in the transmission of nociceptive stimuli from the periphery into the spinal marrow (Passmore et al., J. Neurosci. 2003; 23(18): 7227-36).

It has accordingly been possible to detect an analgesic activity in preclinical neuropathy and inflammatory pain models for the KCNQ2/3 agonist retigabine (Blackburn-Munro and Jensen, Eur J. Pharmacol. 2003; 460(2-3); 109-16; post et al., Naunyn Schmiedebergs Arch Pharmacol 2004; 369(4): 382-390).

The KCNQ2/3 K⁺ channel thus represents a suitable starting point for the treatment of pain; in particular of pain selected from the group consisting of chronic pain, acute pain, neuropathic pain, inflammatory pain, visceral pain and muscular pain (Nielsen et al., Eur J. Pharmacol. 2004; 487(1-3): 93-103), in particular of neuropathic and inflammatory pain.

Moreover, the KCNQ2/3 K⁺ channel is a suitable target for therapy of a large number of further diseases, such as, for example, migraine (US2002/0128277), cognitive diseases (Gribkoff, Expert Opin Ther Targets 2003; 7(6): 737-748), anxiety (Korsgaard et al., J Pharmacol Exp Ther. 2005, 14(1): 282-92), epilepsy (Wickenden et al., Expert Opin Ther Pat 2004; 14(4): 457-469; Gribkoff, Expert Opin Ther Targets 2008, 12(5): 565-81; Miceli et al., Curr Opin Pharmacol 2008, 8(1): 65-74), urinary incontinence (Streng et al., J Urol 2004; 172: 2054-2058), dependency (Hansen et al., Eur J Pharmacol 2007, 570(1-3): 77-88), mania/bipolar disorders (Dencker et al., Epilepsy Behav 2008, 12(1): 49-53) and dystonia-associated dyskinesias (Richter et al., Br J Pharmacol 2006, 149(6): 747-53).

Substituted compounds that have an affinity for the KCNQ2/3 K⁺ channel are e.g. known from the prior art (WO 2012/052167, WO 2008/046582, WO 2010/046108, WO 2010/102809 and WO 2002/066036).

There is a demand for further compounds having comparable or better properties, not only with regard to affinity to KCNQ2/3 K⁺ channels per se (potency, efficacy).

Thus, it may be advantageous to improve the metabolic stability, the solubility in aqueous media or the permeability of the compounds. These factors can have a beneficial effect on oral bioavailability or can alter the PK/PD (pharmacokinetic/pharmacodynamic) profile; this can lead to a more beneficial period of effectiveness, for example. A weak or non-existent interaction with transporter molecules, which are involved in the ingestion and the excretion of pharmaceutical compositions, is also to be regarded as an indication of improved bioavailability and at most low interactions of pharmaceutical compositions. Furthermore, the interactions with the enzymes involved in the decomposition and the excretion of pharmaceutical compositions should also be as low as possible, as such test results also suggest that at most low interactions, or no interactions at all, of pharmaceutical compositions are to be expected.

In addition, it may be advantageous if the compounds show a high selectivity towards other receptors of the KCNQ family (specificity), e.g. towards KCNQ1, KCNQ3/5 or KCNQ4. A high selectivity may have a positive effect on the side effects profile: for example it is known that compounds which (also) have an affinity to KCNQ1 are likely to have a potential for cardial side effects. Therefore, a high selectivity towards KCNQ1 may be desirable. However, it may also be advantageous for the compounds to show a high selectivity towards other receptors. For instance, it may be advantageous for the compounds to show a low affinity for the hERG ion channel or the L-type calcium ion channel (phenylalkylamine-, benzothiazepin-, dihydropyridine-binding site) since these receptors are known to possibly have a potential for cardial side effects. Further, an improved selectivity towards binding to other endogenic proteins (i.e. receptors or enzymes) may result in a better side effects profile and, consequently to an improved tolerance.

It was therefore an object of the invention to provide new compounds having advantages over the compounds of the prior art. These compounds should be suitable in particular as pharmacological active ingredients in pharmaceutical compositions, preferably in pharmaceutical compositions for the treatment and/or prophylaxis of disorders and/or diseases which are mediated, at least in part, by KCNQ2/3 K⁺ channels.

That object is achieved by the subject-matter described herein.

It has been found, surprisingly, that compounds of the general formula (I) given below are suitable for the treatment of pain. It has also been found, surprisingly, that substituted compounds of the general formula (I) given below also have an excellent affinity for the KCNQ2/3 K⁺ channel and are therefore suitable for the prophylaxis and/or treatment of disorders and/or diseases that are mediated at least in part by KCNQ2/3 K⁺ channels. The substituted compounds thereby act as modulators, i.e. agonists or antagonists, of the KCNQ2/3 K⁺ channel.

The present invention therefore relates to a compound of general formula (I),

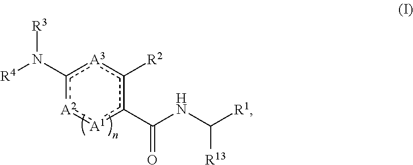

wherein
A¹ represents CR⁵ or N;
A² represents CR⁶, N, O, S or NR⁷;
A³ represents CR⁸ or N, and
n denotes 0 or 1,
   on the condition, that
   if n denotes 0, then A² represents O, S or NIR⁷, or
   if n denotes 1, then A² represents CR⁶ or N,
      wherein
R⁵ is selected from F, Cl, Br, CN, CH₃, CF₃, CHF₂, CH₂F, OCH₃, C₂H₅, SCH₃, OCF₃, OCHF₂ or OCH₂F;
R⁶ is selected from H, F, Cl, Br, CN, CH₃, CF₃, CHF₂, CH₂F, OCH₃, C₂H₅, SCH₃, OCF₃, OCHF₂ or OCH₂F;
R⁷ represents C₁₋₄-aliphatic residue or C₃₋₅-cycloaliphatic residue, in each case unsubstituted or mono- or polysubstituted;
R⁸ is selected from H, F, Cl, Br, CN, CH₃, CF₃, CHF₂, CH₂F, OCH₃, C₂H₅, SCH₃, OCF₃, OCHF₂ or OCH₂F;
   with the proviso, that,
      if n denotes 1, then at least one of A¹, A² and A³ denotes N,
   with the proviso, that
      if n denotes 1 and A³ denotes N, then A¹ and/or A² denotes N,
   and with the proviso, that
      if n denotes 1 and A² denotes N and A¹ denotes CR⁵ and A³ denotes CR⁸, then R⁵ denotes F, Cl, CH₃, CF₃, CHF₂ or CH₂F;
R¹³ represents H or C₁₋₄-aliphatic residue,
R¹ represents
   C₁₋₁₀-aliphatic residue, unsubstituted or mono- or polysubstituted; or
   C₃₋₁₀-cycloaliphatic residue or 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted;
   or
   aryl or heteroaryl, in each case unsubstituted or mono- or polysubstituted and in each case optionally linked via a C₁₋₄-aliphatic group, which in turn may be unsubstituted or mono- or polysubstituted;
R² represents C₁₋₆-aliphatic residue, unsubstituted or mono- or polysubstituted; a C₃₋₆-cycloaliphatic residue or a 3 to 6 membered heterocycloaliphatic residue, in each case unsubstituted or mono- or polysubstituted,
   or
   denotes S—R⁹, O—R¹⁰ or N(R¹¹R¹²),
      wherein
      R⁹ and R¹⁰ in each case represent C₁₋₆-aliphatic residue, unsubstituted or mono- or polysubstituted; C₃₋₆-cycloaliphatic residue or 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted;
         with the proviso, that if R⁹ or R¹⁰ denote a 3 to 7 membered heterocycloaliphatic residue, than the 3 to 7 membered heterocycloaliphatic residue is linked via a carbon atom,
      R¹¹ represents C₁₋₆-aliphatic residue, unsubstituted or mono- or polysubstituted; C₃₋₆-cycloaliphatic residue or a 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted;
         with the proviso that if R¹¹ denotes a 3 to 7 membered heterocycloaliphatic residue, the 3 to 7 membered heterocycloaliphatic residue is linked via a carbon atom; and
      R¹² denotes C₁₋₆-aliphatic residue, unsubstituted or mono- or polysubstituted;
      or
      R¹¹ and R¹² form together with the nitrogen atom connecting them a 3 to 7 membered heterocycloaliphatic residue, unsubstituted or mono- or polysubstituted;
R³ represents C₁₋₁₀-aliphatic residue, unsubstituted or mono- or polysubstituted; or
   C₃₋₁₀-cycloaliphatic residue or 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted;
   or
   aryl or heteroaryl, in each case unsubstituted or mono- or polysubstituted and in each case optionally bridged via a C₁₋₈-aliphatic group, which in turn may be unsubstituted or mono- or polysubstituted;
      on the condition that if R³ denotes a 3 to 10 membered heterocycloaliphatic residue or a heteroaryl, the 3 to 10 membered heterocycloaliphatic residue or the heteroaryl is linked via a carbon atom;
and
R⁴ denotes H or C₁₋₁₀-aliphatic residue, unsubstituted or mono- or polysubstituted;
   or
R³ and R⁴ form together with the nitrogen atom connecting them a 3 to 10 membered heterocycloaliphatic residue, unsubstituted or mono- or polysubstituted;
   in which an “aliphatic group” and “aliphatic residue” may in each case be branched or unbranched, saturated or unsaturated,
   in which a “cycloaliphatic residue” and a “heterocycloaliphatic residue” may in each case be saturated or unsaturated,
   in which “mono- or polysubstituted” with respect to “aliphatic group”, “aliphatic residue”, “cycloaliphatic residue” and “heterocycloaliphatic residue” relates, with respect to the corresponding residues or groups, to the substitution of one or more hydrogen atoms each independently of one another by at least one substituent selected from the group consisting of F, Cl, Br, I, NO₂, NH₂, NH(C₁₋₄-aliphatic residue), N(C₁₋₄-aliphatic residue)₂, NH—C(═O)—C₁₋₄-aliphatic residue, N(C₁₋₄ aliphatic residue)-C(═O)—C₁₋₄-aliphatic residue, NH—S(═O)₂—C₁₋₄-aliphatic residue, N(C₁₋₄-aliphatic residue)-S(═O)₂—C₁₋₄ aliphatic residue, ═O, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂—C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, CHO, COOH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, C(═O)NH₂, a C(═O)—NH(C₁₋₄-aliphatic residue) and C(═O)—N(C₁₋₄-aliphatic residue)₂;
   in which “mono- or polysubstituted” with respect to “aryl” and “heteroaryl” relates, with respect to the corresponding residues, to the substitution of one or more hydrogen atoms each independently of one another by at least one substituent selected from the group consisting of F, Cl, Br, I, NO₂, NH₂,

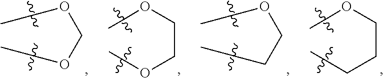

NH(C₁₋₄-aliphatic residue), N(C₁₋₄-aliphatic residue)₂, NH—C(═O)—C₁₋₄-aliphatic residue, N(C₁₋₄ aliphatic residue)-C(═O)—C₁₋₄ aliphatic residue, NH—S(═O)₂—C₁₋₄ aliphatic residue, N(C₁₋₄ aliphatic residue)-S(═O)₂—C₁₋₄ aliphatic residue, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂—C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, C(═O)H, C(═O)OH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, C(═O)—NH(C₁₋₄-aliphatic residue) and C(═O)—N(C₁₋₄-aliphatic residue)₂;
in the form of an individual single stereoisomer or a mixture of the stereoisomers in any mixing ratio, and/or in the form of a free compound, a solvate and/or a physiologically acceptable salt.

Within the scope of this invention, the terms “aliphatic residue” or “aliphatic group” include acyclic saturated or unsaturated aliphatic hydrocarbon radicals, which can be branched or unbranched as well as unsubstituted or mono- or poly-substituted, having from 1 to 10 or from 1 to 8 or from 1 to 6 or from 1 to 4 or from 1 to 2 or from 2 to 6 carbon atoms, that is to say C₁₋₁₀-alkanyls, C₂₋₁₀-alkenyls and C₂₋₁₀-alkynyls or C₁₋₈-alkanyls, C₂₋₈-alkenyls and C₂₋₈-alkynyls or C₁₋₆-alkanyls, C₂₋₆-alkenyls and C₂₋₆-alkynyls or C₁₋₄-alkanyls, C₂₋₄-alkenyls and C₂₋₄-alkynyls or C₁₋₂-alkanyls, C₂-alkenyls and C₂-alkynyls or C₂₋₆-alkanyls, C₂₋₆-alkenyls and C₂₋₆-alkynyls. Alkenyls contain at least one C—C double bond and alkynyls contain at least one C—C triple bond. Alkyl is preferably selected from the group comprising methyl, ethyl, n-propyl, 2-propyl, n-butyl, isobutyl, sec-butyl, tert-butyl, n-pentyl, isopentyl, neopentyl, n-hexyl, n-heptyl, n-octyl, n-nonyl, n-decyl, ethenyl (vinyl), ethynyl, propenyl (—CH₂CH═CH₂, —CH═CHCH₃, —C(═CH₂)CH₃), propynyl CH₂C≡CH, —C≡CCH₃), butenyl, butynyl, pentenyl, pentynyl, hexenyl and hexynyl, heptenyl, heptynyl, octenyl, octynyl, nonenyl, nonynyl, decenyl and decynyl.

For the purposes of this invention, the terms “cycloaliphatic residue” or “C₃₋₁₀-cycloaliphatic residue”, “C₃₋₈-cycloaliphatic residue” and “C₃₋₆-cycloaliphatic residue” denote cyclic aliphatic hydrocarbons having 3, 4, 5, 6, 7, 8, 9 or 10 carbon atoms or having 3, 4, 5, 6, 7 or 8 carbon atoms or having 3, 4, 5 or 6 carbon atoms, wherein the hydrocarbons can be saturated or unsaturated (but not aromatic), unsubstituted or mono- or poly-substituted. The bonding of the cycloaliphatic residue to the general structure of higher order can take place via any desired and possible ring member of the cycloalkyl radical. The cycloaliphatic residue can also be fused with further saturated, (partially) unsaturated, (hetero) cycloaliphatic, aromatic or heteroaromatic ring systems, that is to say with cycloaliphatic residue, heterocycloaliphatic residue, aryl or heteroaryl, which can themselves be unsubstituted or mono- or poly-substituted. The cycloaliphatic residue radicals can further be bridged one or more times, as, for example, in the case of adamantyl, bicyclo[2.2.1]heptyl or bicyclo[2.2.2]octyl. Cycloalkyl is preferably selected from the group comprising cyclopropyl, cyclobutyl, cyclopentyl, cyclopentenyl, cyclohexyl, cyclohexenyl, cycloheptyl, cycloheptenyl, cyclooctyl, cyclooctenyl cyclononyl, cyclodecyl, adamantyl as well as

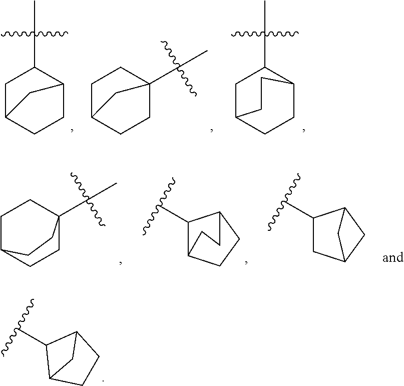

The term “3 to 10 membered heterocycloaliphatic residue” or “3 to 7 membered heterocycloaliphatic residue” or “heterocycloaliphatic residue” includes aliphatic saturated or unsaturated (but not aromatic) heterocycloaliphatic residues having preferenially from three to ten, that is to say 3, 4, 5, 6, 7, 8, 9 or 10, ring members or from three to seven, that is to say 3, 4, 5, 6 or 7, ring members in which at least one carbon atom, optionally also two or three carbon atoms, has been replaced by a heteroatom or heteroatom group in each case selected independently of one another from the group consisting of O, S, S(═O), S(═O)₂, N, NH and N(C₁₋₈alkyl), preferably N(CH₃), wherein the ring members can be unsubstituted or mono- or poly-substituted. The bonding of the heterocycloaliphatic residue to the general structure of higher order can take place via any desired and possible ring member of the heterocycloaliphatic residue. The heterocycloaliphatic residues can also be fused with further saturated, (partially) unsaturated (hetero)cycloaliphatic or aromatic or heteroaromatic ring systems, that is to say with cycloaliphatic residue, heterocycloaliphatic residue, aryl or heteroaryl, which can themselves be unsubstituted or mono- or poly-substituted. The term “fused” also optionally includes spirocycles, i.e. an at least bicyclic ring system, wherein the heterocycloaliphatic residue is connected through just one (spiro) atom with a further saturated or (partially) unsaturated (hetero)cycloaliphatic ring system. Example of such spirocycles are e.g.

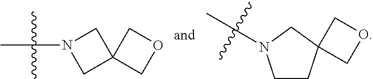 [SMILES: *N1CC2(COC2)C1 ; "and" ; *N1CCC2(COC2)C1]

The heterocycloaliphatic residues can furthermore optionally be singly or multiply bridged with a C₁ or C₂-aliphatic group such as, for example, in the case of

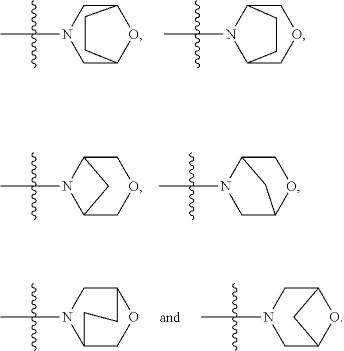

Preferred heterocycloaliphatic residues are selected from the group consisting of azetidinyl, aziridinyl, azepanyl, azocanyl, diazepanyl, dithiolanyl, dihydroquinolinyl, dihydropyrrolyl, dioxanyl, dioxolanyl, dioxepanyl, dihydroindenyl, dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, oxazepanyl, pyrrolidinyl, piperazinyl, 4-methylpiperazinyl, piperidinyl, pyrazolidinyl, pyranyl, tetrahydropyrrolyl, tetrahydropyranyl, tetrahydroquinolinyl, tetrahydroisoquinolinyl, tetrahydroindolinyl, tetrahydrofuranyl, tetrahydropyridinyl, tetrahydrothiophenyl, tetrahydropyridoindolyl, tetrahydronaphthyl, tetrahydrocarbolinyl, tetrahydro-isoxazolo-pyridinyl, thiazolidinyl, tetrahydroimidazo[1,2-a]pyrazinyl, octahydropyrrolo[1,2-a]pyrazinyl and thiomorpholinyl. More preferred heterocycloaliphatic residues are pyrrolidinyl, piperidinyl, oxazepanyl, azetidinyl, morpholinyl, piperazinyl, tetrahydroquinolinyl, tetrahydroisoquinolinyl, dihydroindolinyl, and dihydroisoindolyl. Most preferred heterocycloaliphatic residues are pyrrolidinyl, piperidinyl, oxazepanyl, azetidinyl, tetrahydroquinolinyl, tetrahydroisoquinolinyl, dihydroindolinyl, and dihydroisoindolyl.

Within the scope of this invention, the term “aryl” denotes aromatic hydrocarbons having up to 14 ring members, inter alia phenyls and naphthyls. Each aryl radical can be unsubstituted or mono- or poly-substituted, it being possible for the aryl substituents to be identical or different and to be in any desired and possible position of the aryl. The aryl can be bonded to the general structure of higher order via any desired and possible ring member of the aryl radical. The aryl radicals can also be fused with further saturated, (partially) unsaturated, (hetero)cycloaliphatic, aromatic or heteroaromatic ring systems, that is to say with cycloaliphatic residue, heterocycloaliphatic residue, aryl or heteroaryl, which can themselves be unsubstituted or mono- or poly-substituted. Examples of fused aryl radicals are benzodioxolanyl and benzodioxanyl. Aryl is preferably selected from the group containing phenyl, 1-naphthyl and 2-naphthyl, each of which can be unsubstituted or mono- or poly-substituted. A particularly preferred aryl is phenyl, unsubstituted or mono- or poly-substituted.

The term “heteroaryl” for the purpose of this invention represents a 5 or 6-membered cyclic aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted; in the case of substitution on the heteroaryl, the substituents can be the same or different and be in any desired and possible position of the heteroaryl. The binding to the superordinate general structure can be carried out via any desired and possible ring member of the heteroaryl residue. The heteroaryl can also be part of a bi- or polycyclic system having up to 14 ring members, wherein the ring system can be formed with further saturated, (partially) unsaturated, (hetero)cycloaliphatic or aromatic or heteroaromatic rings, i.e. with a cycloaliphatic, heterocycloaliphatic, aryl or heteroaryl residue, which can in turn be unsubstituted or mono- or polysubstituted. Preferably, the heteroaryl residue is selected from the group consisting of benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl. Furyl, pyridyl, oxazolyl, thiazolyl and thienyl are particularly preferred.

Within the scope of the invention, the expressions “linked via C₁₋₄-aliphatic group” in relation to aryl, heteroaryl, heterocycloaliphatic residue or cycloaliphatic residue is understood that C₁₋₄-aliphatic group and aryl or heteroaryl or heterocycloaliphatic residue or cycloaliphatic residue have the meanings defined above and the aryl or heteroaryl or heterocycloaliphatic residue or cycloaliphatic residue is bonded to the general structure of higher order via a C₁₋₄-aliphatic group. The aliphatic group can in all cases be saturated or unsaturated, branched or unbranched, unsubstituted or mono- or poly-substituted. The C₁₋₄-aliphatic group is preferably selected from C₁₋₄-alkyl groups, preferably from the group comprising of —CH₂—, —CH₂CH₂—, —CH(CH₃)—, —CH₂CH₂CH₂—, —CH(CH₃)CH₂—, —CH(CH₂CH₃)—, —CH₂(CH₂)₂CH₂—, —CH(CH₃)CH₂CH₂—, —CH₂CH(CH₃)CH₂—, —CH(CH₃CH(CH₃)—, —CH(CH₂CH₃)CH₂—, —C(CH₃)₂CH₂—, —CH(CH₂CH₂CH₃)—, —C(CH₃)(CH₂CH₃)—, —CH═CH—, —CH═CHCH₂—, —C(CH₃)═CH₂—, —CH═CHCH₂CH₂—, —CH₂CH═CHCH₂—, —CH═CHCH═CH—, —C(CH₃)═CHCH₂—, —CH═C(CH₃)CH₂—, —C(CH₃)═C(CH₃)—, —C(CH₂CH₃)═CH—, —C≡C—, —C≡CCH₂—, —C≡CCH₂CH₂—, —C≡CCH(CH₃)—, —CH₂C≡CCH₂— and —C≡CC(CH₃)₂—.

In relation with “aliphatic residue”, “aliphatic group”, “heterocycloaliphatic residue” and “cycloaliphatic residue”, the expression “mono- or poly-substituted” is understood as meaning within the scope of this invention the substitution of one or more hydrogen atoms one or more times, for example two, three or four times, in each case independently of one another, by substituents selected from the group comprising F, Cl, Br, I, NO₂, NH₂, NH(C₁₋₄-aliphatic residue), N(C₁₋₄-aliphatic residue)₂, NH—C(═O)—C₁₋₄ aliphatic residue, N(C₁₋₄-aliphatic residue)-C(═O)—C₁₋₄ aliphatic residue, NH—S(═O)₂—C₁₋₄-aliphatic residue, N(C₁₋₄-aliphatic residue)-S(═O)₂—C₁₋₄-aliphatic residue, ═O, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂—C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, CHO, COOH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, a C(═O)—NH(C₁₋₄-aliphatic residue) and C(═O)—N(C₁₋₄-aliphatic residue)₂; wherein polysubstituted radicals are to be understood as being radicals that are substituted several times, for example two, three or four times, either on different atoms or on the same atom, for example three times on the same carbon atom, as in the case of CF₃ or CH₂CF₃, or at different places, as in the case of CH(OH)—CH═CH—CHCl₂. A substituent can itself optionally be mono- or poly-substituted. Polysubstitution may take place with the same or with different substituents.

Preferred substituents of “aliphatic residue”, “aliphatic group”, “heterocycloaliphatic residue” or “cycloaliphatic residue” are selected from the group comprising F, Cl, Br, NH₂, NH(C₁₋₄-aliphatic residue), N(C₁₋₄-aliphatic residue)₂, NH—C(═O)—C₁₋₄-aliphatic residue, NH—S(═O)₂—C₁₋₄-aliphatic residue, ═O, OH, OCF₃, O—C₁₋₄-aliphatic residue, O—C(═O)—C₁₋₄-aliphatic residue, S(═O)₂—C₁₋₄-aliphatic residue, S(═O)₂—NH(C₁₋₄-aliphatic residue), S(═O)₂—N(C₁₋₄-aliphatic residue)₂, CN, CF₃, COOH, C₁₋₄-aliphatic residue, C(═O)—C₁₋₄-aliphatic residue, C₃₋₆-cycloaliphatic residue, 3 to 7 membered heterocycloaliphatic residue, C(═O)NH₂, C(═O)—NH(C₁₋₄-aliphatic residue) and C(═O)—N(C₁₋₄-aliphatic residue)₂.

In relation with “aryl” and “heteroaryl”, the term “mono- or poly-substituted” is understood within the scope of this invention as meaning the substitution of one or more hydrogen atoms of the ring system one or more times, for example two, three or four times, in each case independently of one another, by substituents selected from the group comprising F, Cl, Br, I, NO₂, NH₂,

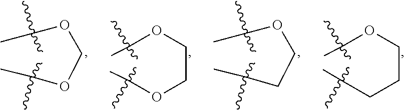 [SMILES: *OCO* ; *OCCO* ; *CCO* ; *CCCO*]

NH(C₁₋₄-aliphatic residue), N(C₁₋₄-aliphatic residue)₂, NH—C(═O)—C₁₋₄-aliphatic residue, N(C₁₋₄-aliphatic residue)-C(═O)—C₁₋₄-aliphatic residue, NH—S(═O)₂—C₁₋₄-aliphatic residue, N(C₁₋₄-aliphatic residue)-S(═O)₂—C₁₋₄-aliphatic residue, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, C(═O)H, C(═O)OH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, C(═O)—NH(C₁₋₄-aliphatic residue) and C(═O)—N(C₁₋₄-aliphatic residue)₂; on one atom or optionally on different atoms, wherein a substituent can itself optionally be mono- or poly-substituted. Polysubstitution is carried out with the same or with different substituents.

Preferred “aryl” and “heteroaryl” substituents are F; Cl; Br; CF₃; CN; C₁₋₄-aliphatic residue; phenyl; naphthyl; pyridyl; thienyl; furyl; C₃₋₆-cycloaliphatic residue; 3 to 7 membered heterocycloaliphatic residue; C(═O)—C₁₋₄-aliphatic residue; CO₂H; C(═O)—O—C₁₋₄-aliphatic residue; CONH₂; C(═O)—NH(C₁₋₄-aliphatic residue); C(═O)—N(C₁₋₄-aliphatic residue)₂; OH; O—C₁₋₄-aliphatic residue; OCF₃; O—C(═O)—C₁₋₄-aliphatic residue; NH₂; NH(C₁₋₄-aliphatic residue); N(C₁₋₄-aliphatic residue)₂; N(H)O(═O)—C₁₋₄-aliphatic residue; S—C₁₋₈-alkyl; SCF₃; S(═O)₂C₁₋₄-aliphatic residue; S(═O)₂—N(H)C₁₋₄-aliphatic residue.

The compounds according to the invention are defined by substituents, for example by R^{A}, R^{B} and R^{C} (1st generation substituents), which are themselves optionally substituted (2nd generation substituents). Depending on the definition, these substituents of the substituents can in turn themselves be substituted (3rd generation substituents). If, for example, R^{A}=aryl (1st generation substituent), aryl can itself be substituted, for example by C₁₋₄-aliphatic residue (2nd generation substituent). This yields the functional group aryl-C₁₋₄-aliphatic residue. C₁₋₄-aliphatic residue can then in turn itself be substituted, for example by Cl (3rd generation substituent). Overall, this then yields the functional group aryl-C₁₋₄-aliphatic residue-Cl.

In a preferred embodiment, however, the 3rd generation substituents cannot themselves be substituted, that is to say there are no 4th generation substituents. In another preferred embodiment, the 2^{nd} generation substituents may not be resubstituted, i.e. there are then not even any 3^{rd} generation substituents. In other words, in this embodiment, in the case of general formula (I), for example, the functional groups for R¹ to R¹³ can each if appropriate be substituted; however, the respective substituents may then for their part not be resubstituted.

In some cases, the compounds according to the invention are defined by substituents which are or carry an aryl or heteroaryl residue, respectively unsubstituted or mono- or polysubstituted, or which form together with the carbon atom(s) or heteroatom(s) connecting them, as the ring member or as the ring members, a ring, for example an aryl or heteroaryl, in each case unsubstituted or mono- or polysubstituted. Both these aryl or heteroaryl residues and the (hetero)aromatic ring systems formed in this way can if appropriate be condensed with a cycloaliphatic, preferably a C₃₋₆ cycloaliphatic residue, or heterocycloaliphatic residue, preferably a 3 to 7 membered heterocycloaliphatic residue, or with aryl or heteroaryl, e.g. with a C₃₋₆ cycloaliphatic residue such as cyclopentyl, or a 3 to 7 membered heterocycloaliphatic residue such as morpholinyl, or an aryl such as phenyl, or a heteroaryl such as pyridyl, wherein the cycloaliphatic or heterocycloaliphatic residues, aryl or heteroaryl residues condensed in this way can for their part be respectively unsubstituted or mono- or polysubstituted.

In some cases, the compounds according to the invention are defined by substituents which are or carry a heterocycloaliphatic residue or heterocycloaliphatic residue, in each case unsubstituted or mono- or poly-substituted, or which, together with the carbon atom(s) or heteroatom(s) joining them as ring member(s), form a ring, for example a cycloaliphatic residue or heterocycloaliphatic residue, in each case unsubstituted or mono- or poly-substituted. Both these cycloaliphatic or heterocycloaliphatic residue and the aliphatic ring systems formed can optionally be fused with aryl or heteroaryl, that is to say with an aryl such as phenyl or with a heteroaryl such as pyridyl, it being possible for the aryl or heteroaryl radicals so fused to be unsubstituted or mono- or poly-substituted.

Within the scope of the present invention, the symbol

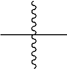

used in the formulae denotes a link of a corresponding residue to the respective superordinate general structure.

The expression “salt formed with a physiologically acceptable acid” is understood within the scope of this invention as meaning salts of the active ingredient in question with inorganic or organic acids that are physiologically acceptable—in particular when used in humans and/or mammals. The hydrochloride is particularly preferred.

Physiologically acceptable salts with cations or bases are salts of the compound in question—in the form of the anion with at least one, preferably inorganic cation—that are physiologically acceptable—in particular when used in humans and/or mammals.

The present invention further relates to a compound of general formula (I),

wherein
A¹ represents CR⁵ or N;
A² represents CR⁶, N, O, S or NR⁷;
A³ represents CR⁸ or N, and
n denotes 0 or 1,
   on the condition, that
   if n denotes 0, then A² represents O, S or NR⁷, or
   if n denotes 1, then A² represents CR⁶ or N,
      wherein
R⁵ is selected from F, Cl, Br, CN, CH₃, CF₃, CHF₂, CH₂F, OCH₃, C₂H₅, SCH₃, OCF₃, OCHF₂ or OCH₂F;
R⁶ is selected from H, F, Cl, Br, CN, CH₃, CF₃, CHF₂, CH₂F, OCH₃, C₂H₅, SCH₃, OCF₃, OCHF₂ or OCH₂F;
R⁷ represents C₁₋₄-aliphatic residue or C₃₋₅-cycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
R⁸ is selected from H, F, Cl, Br, CN, CH₃, CF₃, CHF₂, CH₂F, OCH₃, C₂H₅, SCH₃, OCF₃, OCHF₂ or OCH₂F;
   with the proviso, that,
      if n denotes 1, then at least one of A¹, A² and A³ denotes N,
   with the proviso, that
      if n denotes 1 and A³ denotes N, then A¹ and/or A² denotes N,
   and with the proviso, that
      if n denotes 1 and A² denotes N and A¹ denotes CR⁵ and A³ denotes CR⁸, then R⁵ denotes F, Cl, CH₃, CF₃, CHF₂ or CH₂F;
R¹³ represents H or C₁₋₄-aliphatic residue,
R¹ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH,
   and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄ aliphatic residue, OCF₃, SH, SCF₃, S—C₁₋₄-aliphatic residue, CF₃, CN, C₁₋₄-aliphatic residue and C(═O)OH,
   or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, C₃₋₆-cycloaliphatic residue, a 3 to 7 membered heterocycloaliphatic residue,

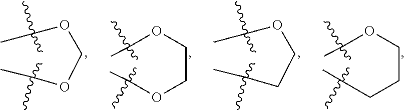 [SMILES: *OCO* ; *OCCO* ; *CCO* ; *CCCO*]

benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, a C₁₋₄-aliphatic residue, C(═O)OH, C(═O)CH₃, C(═O)C₂H₅, C(═O)—OCH₃ and C(═O)—OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, an NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O)OH,
R² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄aliphatic residue,
   or
   denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   or
   denotes S—R⁹, O—R¹⁰ or N(R¹¹R¹²);
   wherein
   R⁹ and R¹⁰ in each case represent C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
      wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or in each case represent C₃₋₆-cycloaliphatic residue or 3 to 7 membered heterocyclo-aliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
      wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
      wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   on the condition that if R⁹ or R¹⁰ denote a 3 to 7 membered heterocycloaliphatic residue, the 3 to 7 membered heterocycloaliphatic residue is linked via a carbon atom,
R¹¹ denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue, and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C(═O)—O—C₁₋₄-aliphatic residue, C₃₋₆-cycloaliphatic residue, and a 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH, and
   wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O)OH,
   on the condition that if R¹¹ denotes a 3 to 7 membered heterocycloaliphatic residue, the 3 to 7 membered heterocycloaliphatic residue is linked via a carbon atom,
and
R¹² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or
R¹¹ and R¹² form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
   wherein the 3 to 10 membered heterocycloaliphatic residue formed by R¹¹ and R¹² together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)—OH, C(═O)—CH₃, C(═O)—C₂H₅, C(═O)—OCH₃ and C(═O)—OC₂H₅, C₃₋₆-cycloaliphatic residue, 3 to 7 membered heterocycloaliphatic residue,

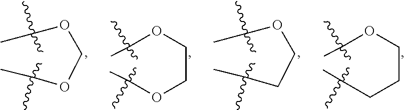 [SMILES: *OCO* ; *OCCO* ; *CCO* ; *CCCO*]

benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
R³ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C(═O)—O—C₁₋₄-aliphatic residue, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O)OH, on the condition that if R³ denotes a 3 to 10 membered heterocycloaliphatic residue, the 3 to 10 membered heterocycloaliphatic residue is linked via a carbon atom,
   or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

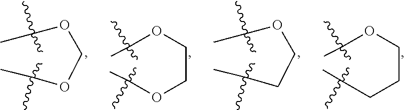 [SMILES: *OCO* ; *OCCO* ; *CCO* ; *CCCO*]

benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O)OH,
R⁴ denotes H or C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄aliphatic residue,
or
R³ and R⁴ form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, ═O, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
   wherein the 3 to 10 membered heterocycloaliphatic residue formed by R³ and R⁴ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

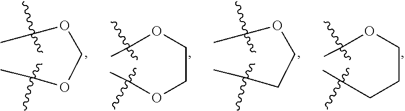 [SMILES: *OCO* ; *OCCO* ; *CCO* ; *CCCO*]

benzyl, phenyl, thienyl, pyridyl, furyl, thiazolyl and oxazolyl,
and wherein the 3 to 10 membered heterocycloaliphatic residue formed by R³ and R⁴ together with the nitrogen atom connecting them may optionally be condensed with a C₃₋₁₀-cycloaliphatic residue or a 3 to 10 membered heterocycloaliphatic residue,
wherein the C₃₋₁₀-cycloaliphatic residue or the 3 to 10 membered heterocycloaliphatic residue condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, ═O, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH.

Within the scope of the present invention, the central structural element of general formula (I)

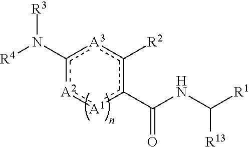

represents a 5-membered or a 6-membered heteroaryl residue. The residue is aromatic as depicted by the dashed bond presentation.

If n represents 1, then central structural element in general formula (I) represents a 6-membered heteroaryl residue (Ia):

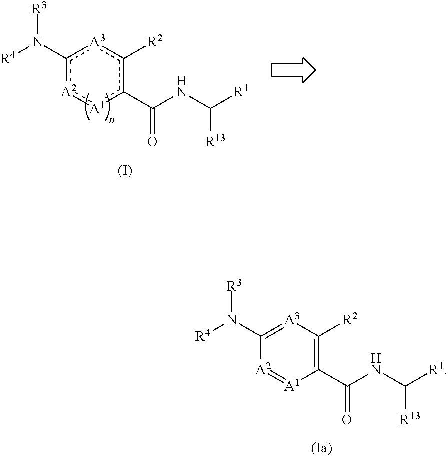

If n represents 0, then the partial structure in general formula (I) represents a 5-membered heteroaryl residue (Ib) or (Ic):

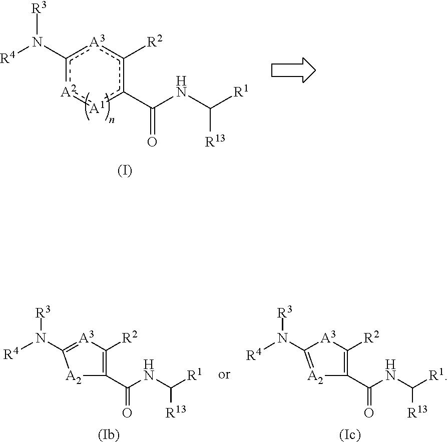

In one embodiment of the present invention, the compound according to general formula (I) is characterized in that

n denotes 0 and the compound is represented by general formula (Ib).

In one preferred embodiment of the present invention, the compound according to general formula (I) is characterized in that
n denotes 0 and the compound is represented by general formula (Ib),
wherein
A² represents O and A³ represents CR⁸ (Ib-1)

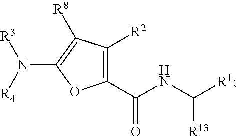

or
A² represents S and A³ represents CR⁸ (Ib-2)

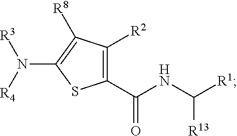

or
A² represents NR⁷ and A³ represents CR⁸ (Ib-3)

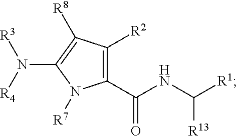

or
A² represents O and A³ represents N (Ib-4)

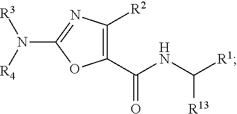

or
A² represents S and A³ represents N (Ib-5)

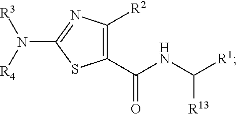

or
A² represents NR⁷ and A³ represents N (Ib-6)

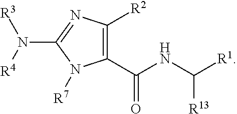

In one particularly preferred embodiment, the compound according to general formula (I) is characterized in that
n denotes 0 and the compound is represented by general formula,
   wherein
A² represents O and A³ represents N (Ib-4);
   or
A² represents S and A³ represents N (Ib-5);
   or
A² represents NR⁷ and A³ represents N (Ib-6).

In another embodiment of the present invention, the compound according to general formula (I) is characterized in that
n denotes 1 and the compound is represented by general formula (Ia), wherein
A¹ represents N and A² represents CR⁶ and A³ represents CIR⁸ (Ia-1)

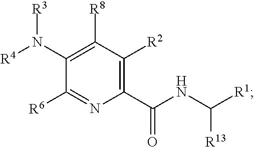

or
A¹ represents CR⁵ and A² represents N and A³ represents CR⁸ (Ia-2)

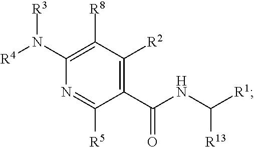

or
A¹ represents N and A² represents N and A³ represents CIR⁸ (Ia-3)

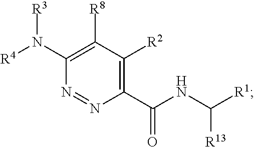

or
A¹ represents N and A² represents CR⁶ and A³ represents N (Ia-4)

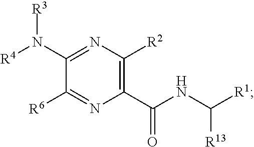

or
A¹ represents CR⁵ and A² represents N and A³ represents N (Ia-5)

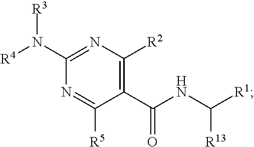

or
A¹ represents N and A² represents N and A³ represents N (Ia-6)

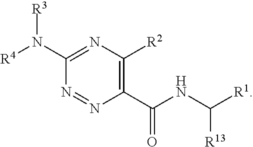

In a preferred embodiment, the compound according to general formula (I) is characterized in that
n denotes 1 and the compound is represented by general formula (Ia), wherein
A¹ represents N and A² represents CR⁶ and A³ represents CR⁸ (Ia-1); or
A¹ represents CR⁵ and A² represents N and A³ represents CR⁸ (Ia-2); or
A¹ represents N and A² represents CR⁶ and A³ represents N (Ia-4); or
A¹ represents CR⁵ and A² represents N and A³ represents N (Ia-5).

Furthermore, the residues R⁵, R⁶, R⁷ and R⁸ are particularly selected.

In another embodiment of the invention, R⁵ denotes F, Cl, CH₃, OCH₃ or CH₂CH₃.

In yet another embodiment of the invention, R⁶ denotes H.

In yet another embodiment of the invention, R⁷ denotes CH₃, CH₂CH₃ or cyclopropyl.

In yet another embodiment of the invention, R⁸ denotes H.

In a preferred embodiment, the compound according to general formulas (I) is characterized in that
R⁵ denotes F, Cl, CH₃, OCH₃ or CH₂CH₃; and/or
R⁶ denotes H; and/or
R⁷ denotes CH₃, CH₂CH₃ or cyclopropyl; and/or
R⁸ denotes H.

In one embodiment of the compound according to general formula (I),
R¹³ represents H or C₁₋₄-aliphatic residue.

In a preferred embodiment of the compound according to general formula (I), R¹³ represents H or CH₃.

In a preferred embodiment of the compound according to general formula (I),
R¹ denotes C₁₋₁₀-aliphatic residue, preferably C₁₋₈-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, S(═O)₂—C₁₋₄-aliphatic residue, CF₃, CN, C₁₋₄-aliphatic residue and C(═O)—OH,
   preferably denotes C₁₋₁₀-aliphatic residue, more preferably a C₁₋₈-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₆ cycloaliphatic residue, and 3 to 7 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
      wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, an NH(C₁₋₄-aliphatic residue), an N(C₁₋₄-aliphatic residue)₂, OH, ═O, an O—C₁₋₄-aliphatic residue, OCF₃, SH, SCF₃, S—C₁₋₄-aliphatic residue, CF₃, CN, C₁₋₄-aliphatic residue and C(═O)OH,
   and wherein the C₃₋₁₀-cycloaliphatic residue or the 3 to 10 membered heterocycloaliphatic residue may in each case optionally linked via a C₁₋₄ aliphatic group, preferably a C₁₋₄ aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, ON, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

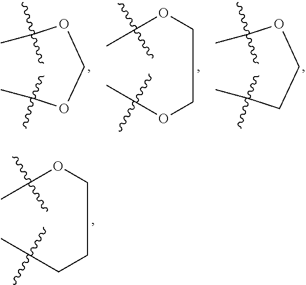 [SMILES: *OCO* ; *OCCO* ; *CCO* ; *CCCO*]

benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄ aliphatic residue, OCF₃, OCH₂CH₂OH, OCH₂OCH₃, SH, SCF₃, S—C₁₋₄-aliphatic residue, CF₃, ON, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O)OH.

In a further embodiment of the invention, the compound according to general formula (I), the residue

R¹ represents the partial structure (T1)

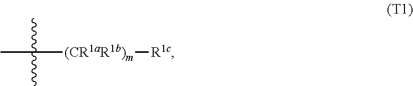

wherein
m denotes 0, 1, 2, 3 or 4, preferably denotes 0, 1, 2 or 3, more preferably denotes 0, 1, or 2,
R^{1a} and R^{1b} each independently of one another represent H, F, Cl, Br, I, NO₂, NH₂, NH(C₁₋₄ aliphatic residue), N(C₁₋₄-aliphatic residue)₂, OH, O—C₁₋₄-aliphatic residue, OCF₃, SH, SCF₃, S—C₁₋₄ aliphatic residue, CF₃, CN, C₁₋₄-aliphatic residue or C(═O)—OH, or together denote ═O,
   preferably each independently of one another represent H, F, Cl, Br, I, NH₂, NH(C₁₋₄-aliphatic residue), N(C₁₋₄-aliphatic residue)₂, OH, O—C₁₋₄-aliphatic residue or C₁₋₄-aliphatic residue, or together denote ═O,
   more preferably each independently of one another represent H, F, Cl, Br, I, OH, O—C₁₋₄-aliphatic residue or C₁₋₄-aliphatic residue, or together denote ═O, even more preferably each independently of one another represent H, F, OH, O—C₁₋₄-aliphatic residue or C₁₋₄-aliphatic residue, or together denote ═O, and
R^{1c} denotes C₁₋₄-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, S(═O)₂—C₁₋₄-aliphatic residue, CF₃, CN, C₁₋₄-aliphatic residue and C(═O)—OH, preferably denotes C₁₋₄-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue, preferably when m is ≠0,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH,
   or denotes
   —preferably when m is 0 or 2, more preferably when m is 0—
   aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

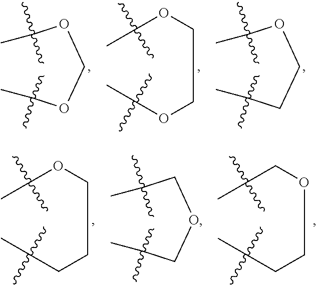 [SMILES: *OCO* ; *OCCO* ; *CCO* ; *CCCO* ; *COC* ; *CCOC*]

benzyl, phenyl, thienyl, pyridyl, furyl, thiazolyl and oxazolyl,
preferably when m is =0,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH.

Preferably,
R¹ represents the partial structure (T1),
wherein
m denotes 0, 1, or 2,
R^{1a} and R^{1b} each independently of one another represent H, F, Cl, Br, I, O—C₁₋₄-aliphatic residue or C₁₋₄-aliphatic residue,
and
R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue,
   wherein the C₁₋₄-aliphatic residue in each case may be unsubstituted or mono- or polysubstituted with at least one substituent selected from the group consisting of F, Cl, CF₃ and O—C₁₋₄-aliphatic residue,
   or
   denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
   wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue,
   or
   denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
   wherein benzyl, phenyl, thienyl and pyridyl may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃C₁₋₄-aliphatic residue and C(═O)OH.

In a further preferred embodiment of the compound according to general formula (I), the residue
R¹ represents the partial structure (T1),
wherein
m is 0, 1 or 2, preferably 0 or 2, more preferably 2, and
R^{1a} and R^{1b} each independently of one another represent H, F, OH, O—C₁₋₄-aliphatic residue or C₁₋₄-aliphatic residue, preferably H, F, OH, CH₃ or OCH₃;
R^{1c} denotes C₁₋₄-aliphatic residue, unsubstituted or mono- or polysubstituted with at least one substituent selected from the group consisting of F, Cl, Br, I, CN, OH, unsubstituted O—C₁₋₄-aliphatic residue, CF₃ and unsubstituted C₁₋₄-aliphatic residue, preferably denotes C₁₋₄-aliphatic residue, unsubstituted or mono- or polysubstituted with at least one substituent selected from the group consisting of F, Cl, Br, I, unsubstituted O—C₁₋₄-aliphatic residue, CF₃, and unsubstituted C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, unsubstituted O—C₁₋₄-aliphatic residue, CF₃, and unsubstituted C₁₋₄-aliphatic residue,
or
wherein
m is 0 or 2, more preferably 0, and
R^{1a} and R^{1b} each independently of one another represent H, F, OH, O—C₁₋₄-aliphatic residue or C₁₋₄-aliphatic residue, preferably H, F, OH, CH₃ or OCH₃; and
R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, OCF₂H, SCF₃, NO₂, N(C₁₋₄-aliphatic residue)₂,

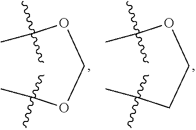 [SMILES: *OCO* ; *CCO*]

CF₃, CN, C₁₋₄-aliphatic residue, C(═O)CH₃, C(═O)C₂H₅, C(═O)OCH₃, C(═O)OC₂H₅ and phenyl,
preferably denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, preferably with at least one substituent selected from the group consisting of F, Cl, CH₃, OCH₃, CF₃ and OCF₃.

Preferably,
R¹ represents the partial structure (T1),
wherein
m denotes 1 or 2,
R^{1a} and R^{1b} represent H,
R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue,
   or
   denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
or
m denotes 0 and
R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
   wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃C₁₋₄-aliphatic residue and C(═O)OH.

In another embodiment of the present invention, the compound according to general formula (I) is characterized in that
R² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or
   denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   or
   denotes S—R⁹, O—R¹⁰ or N(R¹¹R¹²),
   wherein
   R⁹ and R¹⁰ in each case represent C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or in each case represent C₃₋₆-cycloaliphatic residue or 3 to 7 membered heterocyclo-aliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
   wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   on the condition that if R⁹ or R¹⁰ denote a 3 to 7 membered heterocycloaliphatic residue, the 3 to 7 membered heterocycloaliphatic residue is linked via a carbon atom,
   R¹¹ denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue, and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C(═O)—O—C₁₋₄-aliphatic residue, C₃₋₆-cycloaliphatic residue, and a 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH, and
   wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O)OH,
   on the condition that if R¹¹ denotes a 3 to 7 membered heterocycloaliphatic residue, the 3 to 7 membered heterocycloaliphatic residue is linked via a carbon atom,
   and
   R¹² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄aliphatic residue,
   or
   R¹¹ and R¹² form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
   wherein the 3 to 10 membered heterocycloaliphatic residue formed by R¹¹ and R¹² together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)—OH, C(═O)—CH₃, C(═O)—C₂H₅, C(═O)—OCH₃ and C(═O)—OC₂H₅, C₃₋₆-cycloaliphatic residue, 3 to 7 membered heterocycloaliphatic residue,

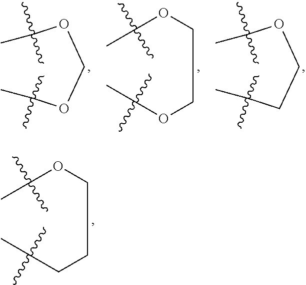 [SMILES: *OCO* ; *OCCO* ; *CCO* ; *CCCO*]

benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH.

Preferably,
R² denotes a C₁₋₆-aliphatic residue, unsubstituted or mono- or polysubstituted with at least one substituent selected from the group consisting of F, Cl, Br, I, NO₂, OH, ═O, O—C₁₋₄-aliphatic residue, OCF₃, SH, SCF₃, S—C₁₋₄-aliphatic residue, CF₃, CN and C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
   or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, OH, ═O, O—C₁₋₄ aliphatic residue, OCF₃, SH, SCF₃, S—C₁₋₄ aliphatic residue, CF₃, CN and C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
   wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, OH, ═O, O—C₁₋₄-aliphatic residue, OCF₃, SCF₃, CF₃CN, and C₁₋₄-aliphatic residue.
or
R² denotes S—R⁹ or O—R¹⁰,
   wherein
   R⁹ and R¹⁰ in each case represent C₁₋₆-aliphatic residue, unsubstituted or mono- or polysubstituted with at least one substituent selected from the group consisting of F, Cl, Br, I, OH, ═O, O—C₁₋₄-aliphatic residue, OCF₃, SH, SCF₃, S—C₁₋₄-aliphatic residue, NH(C₁₋₄-aliphatic residue), N(C₁₋₄-aliphatic residue)₂, CF₃, and C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and unsubstituted O—C₁₋₄-aliphatic residue,
   or in each case denote C₃₋₆-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue,
      wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue,
   and wherein the C₃₋₆-cycloaliphatic residue or the 3 to 7 membered heterocycloaliphatic residue may in each case optionally linked via a C₁₋₄-aliphatic group, preferably a C₁₋₄-aliphatic group, which in turn may be unsubstituted or mono- or polysubstituted with at least one substituent selected from the group consisting of F, Cl, OH, ═O, O—C₁₋₄-aliphatic residue, OCF₃, CF₃, CN, and C₁₋₄-aliphatic residue,
   on the condition that if R⁹ or R¹⁰ denotes a 3 to 7 membered heterocycloaliphatic residue, the 3 to 7 membered heterocycloaliphatic residue is linked via a carbon atom,
or
R² denotes N(R¹¹R¹²),
   wherein
   R¹¹ denotes C₁₋₆-aliphatic residue, unsubstituted or mono- or polysubstituted with at least one substituent selected from the group consisting of F, Cl, Br, I, OH, ═O, O—C₁₋₄-aliphatic residue, OCF₃, SCF₃, CF₃ and C₁₋₄-aliphatic residue,
      wherein the C₁₋₄-aliphatic residue in each case may be unsubstituted or mono- or polysubstituted with at least one substituent selected from the group consisting of F, Cl, OH, CF₃ and unsubstituted O—C₁₋₄-aliphatic residue,
   or denotes a C₃₋₆-cycloaliphatic residue or a 3 to 7 membered heterocycloaliphatic residue, in each case unsubstituted or mono- or polysubstituted with at least one substituent selected from the group consisting of F, Cl, OH, ═O, O—C₁₋₄-aliphatic residue, OCF₃, SCF₃, CF₃, and C₁₋₄-aliphatic residue,
      wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
   and wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, OH, ═O, O—C₁₋₄-aliphatic residue, OCF₃, SCF₃, CF₃, CN and C₁₋₄-aliphatic residue, on the condition that if R¹¹ denotes a 3 to 7 membered heterocycloaliphatic residue, the 3 to 7 membered heterocycloaliphatic residue is linked via a carbon atom,
   and wherein
   R¹² denotes C₁₋₆-aliphatic residue, unsubstituted or mono- or polysubstituted with at least one substituent selected from the group consisting of F, Cl, OH, ═O, O—C₁₋₄-aliphatic residue, OCF₃, SCF₃, CF₃, CN, and C₁₋₄-aliphatic residue,
      wherein the C₁₋₄-aliphatic residue in each case may be unsubstituted or mono- or polysubstituted with at least one substituent selected from the group consisting of F, Cl, OH, CF₃ and unsubstituted O—C₁₋₄-aliphatic residue,
   or
   R¹¹ and R¹² form together with the nitrogen atom connecting them a 3 to 10 membered heterocycloaliphatic residue, preferably a 3 to 7 membered heterocycloaliphatic residue, more preferably selected from the group consisting of morpholinyl, piperidinyl, pyrrolidinyl, azetidinyl and piperazinyl, unsubstituted or mono- or polysubstituted with at least one substituent selected from the group consisting of F, Cl, OH, ═O, O—C₁₋₄-aliphatic residue, OCF₃, SCF₃, CF₃, CN, and C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and unsubstituted O—C₁₋₄-aliphatic residue,
   and wherein the 3 to 10 membered heterocycloaliphatic residue formed by R¹¹ and R¹² together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, OH, O—C₁₋₄-aliphatic residue, OCF₃, SCF₃, CF₃, CN, C₁₋₄-aliphatic residue, C(═O)OH, C₃₋₆ cycloaliphatic residue, a 3 to 7 membered heterocycloaliphatic residue,

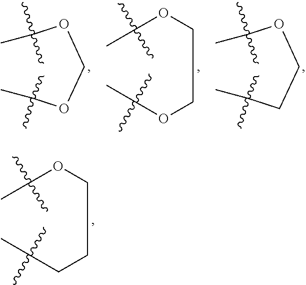 [SMILES: *OCO* ; *OCCO* ; *CCO* ; *CCCO*]

benzyl, phenyl, thienyl, and pyridyl,
wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
wherein benzyl, phenyl, thienyl, and pyridyl, may in each case may be unsubstituted or mono- or polysubstituted with at least one substituent selected from the group consisting of F, Cl, OH, O—C₁₋₄-aliphatic residue, OCF₃, OCH₂CH₂OH, OCH₂OCH₃, SCF₃, CF₃, CN, C₁₋₄-aliphatic residue, and C(═O)OH, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, OH, ═O, O—C₁₋₄-aliphatic residue, OCF₃, CF₃, CN, C₁₋₄-aliphatic residue and C(═O)OH.

More preferably,
R² denotes C₁₋₆-aliphatic residue, unsubstituted or mono- or polysubstituted with at least one substituent selected from the group consisting of F, Cl, OH, ═O, O—C₁₋₄-aliphatic residue, OCF₃, CF₃ and C₁₋₄-aliphatic residue,
   wherein the C₁₋₄-aliphatic residue in each case is unsubstituted,
   or denotes C₃₋₆-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄ aliphatic residue, OCF₃, CF₃ and C₁₋₄-aliphatic residue,
   wherein the C₁₋₄-aliphatic residue in each case may be unsubstituted or mono- or polysubstituted with OH or unsubstituted O—C₁₋₄-aliphatic residue.
   and wherein the C₃₋₆-cycloaliphatic residue or the 3 to 7 membered heterocycloaliphatic residue may in each case optionally linked via a unsubstituted C₁₋₄-aliphatic group,
or
R² denotes S—R⁹ or O—R¹⁰,
   wherein
   R⁹ and R¹⁰ in each case denote C₁₋₆-aliphatic residue, unsubstituted or mono- or polysubstituted with at least one substituent selected from the group consisting of F, Cl, OH, ═O, O—C₁₋₄-aliphatic residue, OCF₃, CF₃ and C₁₋₄-aliphatic residue,
      wherein the C₁₋₄-aliphatic residue in each case may be unsubstituted or mono- or polysubstituted with at least one substituent selected from the group consisting of F, Cl, OH, CF₃ and unsubstituted O—C₁₋₄-aliphatic residue,
   or in each case denote C₃₋₆-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄ aliphatic residue, OCF₃, CF₃, and C₁₋₄-aliphatic residue,
   wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
   wherein the C₃₋₁₀-cycloaliphatic residue or the 3 to 10 membered heterocycloaliphatic residue in each case may be linked, preferably is linked, via an unsubstituted C₁₋₄-aliphatic group, on the condition that if R⁹ or R¹⁰ denotes a 3 to 7 membered heterocycloaliphatic residue, the 3 to 7 membered heterocycloaliphatic residue is linked via a carbon atom,
or
R² denotes N(R¹¹R¹²),
   wherein
   R¹¹ denotes C₁₋₆-aliphatic residue, unsubstituted or mono- or polysubstituted with at least one substituent selected from the group consisting of F, Cl, OH, ═O, O—C₁₋₄-aliphatic residue, OCF₃, CF₃, and C₁₋₄-aliphatic residue
   wherein the C₁₋₄-aliphatic residue in each case may be unsubstituted or mono- or polysubstituted with at least one substituent selected from the group consisting of F, Cl, OH, CF₃ and unsubstituted O—C₁₋₄-aliphatic residue,
   or
   R¹¹ denotes C₃₋₇-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄ aliphatic residue, OCF₃, CF₃, and a C₁₋₄-aliphatic residue,
   wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
   wherein the C₃₋₁₀-cycloaliphatic residue or the 3 to 10 membered heterocycloaliphatic residue may be linked via an unsubstituted C₁₋₄-aliphatic group,
   on the condition that if R¹¹ denotes a 3 to 7 membered heterocycloaliphatic residue, the 3 to 7 membered heterocycloaliphatic residue is linked via a carbon atom,
   and
   R¹² denotes unsubstituted C₁₋₄-aliphatic residue,
   preferably selected from the group consisting of methyl, ethyl, n-propyl, 2-propyl, n-butyl, isobutyl, sec.-butyl and tert.-butyl, more preferably selected from the group consisting of methyl and ethyl,
   or
   R¹¹ and R¹² form together with the nitrogen atom connecting them a 3 to 7 membered heterocycloaliphatic residue, preferably selected from the group consisting of morpholinyl, piperidinyl, pyrrolidinyl, and azetidinyl, unsubstituted or mono- or polysubstituted with at least one substituent selected from the group consisting of F, Cl, OH, ═O, O—C₁₋₄-aliphatic residue, OCF₃, CF₃, CN, and C₁₋₄-aliphatic residue,
      wherein the C₁₋₄-aliphatic residue in each case may be unsubstituted or mono- or polysubstituted with at least one substituent selected from the group consisting of F, Cl, OH, CF₃ and unsubstituted O—C₁₋₄-aliphatic residue,
   and wherein the 3 to 7 membered heterocycloaliphatic residue formed by R¹¹ and R¹² together with the nitrogen atom connecting them may optionally be fused with phenyl or pyridyl, wherein the phenyl or pyridyl residues fused in this way can for their part be respectively unsubstituted or mono- or polysubstituted with at least one substituent selected from the group consisting of F, Cl, OH, O—C₁₋₄-aliphatic residue, OCF₃, CF₃, CN, C₁₋₄-aliphatic residue, benzyl, phenyl, and pyridyl,
      wherein the C₁₋₄-aliphatic residue in each case may be unsubstituted or mono- or polysubstituted with at least one substituent selected from the group consisting of F, Cl, OH, and unsubstituted O—C₁₋₄-aliphatic residue, and
      wherein benzyl, phenyl, and pyridyl, may in each case may be unsubstituted or mono- or polysubstituted with at least one substituent selected from the group consisting of F, Cl, OCH₃, OCF₃, CF₃, and C₁₋₄-aliphatic residue.

More preferably,
R² denotes methyl, ethyl, n-propyl, 2-propyl, n-butyl, isobutyl, sec.-butyl, tert.-butyl, n-pentyl, isopentyl, neopentyl, n-hexyl, ethenyl or propenyl,
   in each case unsubstituted or mono- or polysubstituted with at least one substituent selected from the group consisting of F, Cl, OH, O—C₁₋₄-aliphatic residue, CF₃ and C₁₋₄-aliphatic residue, preferably in each case unsubstituted or mono- or polysubstituted with at least one substituent selected from the group consisting of F, Cl, and an O—C₁₋₄-aliphatic residue, preferably OCH₃, more preferably in each case unsubstituted,
      wherein the C₁₋₄-aliphatic residue in each case is unsubstituted,
   or denotes cyclopropyl, cyclobutyl, cyclopentyl and cyclohexyl, oxetanyl, piperidinyl, tetrahydrofuranyl, or tetrahydropyranyl,
   preferably denotes cyclopropyl or tetrahydropyranyl, more preferably cyclopropyl,
   in each case unsubstituted or mono- or polysubstituted with at least one substituent selected from the group consisting of F, Cl, OH, O—C₁₋₄-aliphatic residue, CF₃ and C₁₋₄-aliphatic residue,
   preferably in each case unsubstituted or mono- or polysubstituted with at least one substituent selected from the group consisting of F, Cl and O—C₁₋₄-aliphatic residue, preferably OCH₃, more preferably in each case unsubstituted,
      wherein the C₁₋₄-aliphatic residue in each case is unsubstituted,
   and wherein cyclopropyl, cyclobutyl, cyclopentyl and cyclohexyl, oxetanyl, piperidinyl, tetrahydrofuranyl, and tetrahydropyranyl may in each case be optionally bridged via an unsubstituted C₁₋₄-aliphatic group, preferably via an unsubstituted C₁₋₂-aliphatic group,
or
R² denotes S—R⁹ or O—R¹⁰,
   wherein
   R⁹ and R¹⁰ in each case denote methyl, ethyl, n-propyl, 2-propyl, n-butyl, isobutyl, sec.-butyl, tert.-butyl, n-pentyl, isopentyl, neopentyl, n-hexyl, ethenyl and propenyl, in each case unsubstituted or mono- or polysubstituted with at least one substituent selected from the group consisting of F, Cl, OH, and O—C₁₋₄-aliphatic residue,
      wherein the C₁₋₄-aliphatic residue in each case is unsubstituted,
   or in each case denote cyclopropyl, cyclobutyl, cyclopentyl cyclohexyl, oxetanyl, piperidinyl, tetrahydrofuranyl, or tetrahydropyranyl, preferably cyclopropyl or oxetanyl, in each case unsubstituted or mono- or polysubstituted with at least one substituent selected from the group consisting of F, Cl, OH, O—C₁₋₄-aliphatic residue, CF₃ and C₁₋₄-aliphatic residue, preferably in each case unsubstituted or mono- or polysubstituted with at least one substituent selected from the group consisting of F, Cl and O—C₁₋₄-aliphatic residue, more preferably in each case unsubstituted,
      wherein the C₁₋₄-aliphatic residue in each case is unsubstituted,
   and wherein cyclopropyl, cyclobutyl, cyclopentyl and cyclohexyl, oxetanyl, piperidinyl, tetrahydrofuranyl, and tetrahydropyranyl may in each case be optionally linked via an unsubstituted C₁₋₄-aliphatic group,
   on the condition that if R⁹ or R¹⁰ denotes piperidinyl, oxetanyl, tetrahydrofuranyl, or tetrahydropyranyl, each of these residues is linked via a carbon atom,
or
R² denotes N(R¹¹R¹²),
   wherein
   R¹¹ denotes C₁₋₆-aliphatic residue,
   unsubstituted or mono- or polysubstituted with at least one substituent selected from the group consisting of F, Cl, ═O, OH and OCH₃,
   preferably unsubstituted or mono- or polysubstituted with at least one substituent selected from the group consisting of F, Cl and OCH₃,
   more preferably unsubstituted or mono- or polysubstituted with at least one substituent selected from the group consisting of F and OCH₃,
   preferably denotes unsubstituted C₁₋₆-aliphatic residue,
   more preferably selected from the group consisting of methyl, ethyl, n-propyl, 2-propyl, n-butyl, isobutyl, sec.-butyl, tert.-butyl, n-pentyl, isopentyl, neopentyl, and n-hexyl,
   and
   R¹² denotes methyl, ethyl, n-propyl, 2-propyl, n-butyl, isobutyl, sec.-butyl or tert.-butyl, more preferably methyl or ethyl,
   or
   R¹¹ and R¹² form together with the nitrogen atom connecting them a morpholinyl, piperidinyl, pyrrolidinyl, or azetidinyl,
   in each case unsubstituted or mono- or polysubstituted with at least one substituent selected from the group consisting of F, Cl, OH, O—C₁₋₄ aliphatic residue and C₁₋₄-aliphatic residue,
   more preferably unsubstituted or mono- or polysubstituted with at least one substituent selected from the group consisting of F, Cl and O—C₁₋₄ aliphatic residue,
   preferably form together with the nitrogen atom connecting them a morpholinyl, piperidinyl, pyrrolidinyl, or azetidinyl, in each case unsubstituted.

In a particular preferred embodiment of the present invention, the compound according to general formula (I) is characterized in that
R² is selected from the group consisting of
   CH₃, C₂H₅, CH₂CH₂CH₃, CH(CH₃)₂, CH₂CH₂CH₂CH₃, CH₂CH(CH₃)₂, CH(CH₃)CH₂CH₃, C(CH₃)₃, CH₂-cyclopropyl, OCH₃, OC₂H₅, OCH₂CH₂CH₃, OCH(CH₃)₂, O-cyclopropyl, SCH₃, SC₂H₅, SCH₂CH₂CH₃, SCH(CH₃)₂, S-cyclopropyl, cyclopropyl, cyclobutyl, cyclopentyl, cyclohexyl, N(CH₃)₂, N(CH₃)C₂H₅, N(CH₃)CH₂CH₂CH₃, N(CH₃)CH(CH₃)₂, N(CH₃)-cyclopropyl, N(C₂H₅)₂, N(C₂H₅)CH₂CH₂CH₃, N(C₂H₅)CH(CH₃)₂, N(C₂H₅)-cyclopropyl, N-aziridinyl, N-azetidinyl, N-pyrrolidinyl, N-piperidinyl or N-morpholinyl,
      in each case unsubstituted or mono- or polysubstituted with F, OH and/or OCH₃.

In a further embodiment of the present invention, the compound according to general formula (I) is characterized in that
R³ denotes a C₁₋₁₀-aliphatic residue, preferably a C₁₋₈-aliphatic residue, unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, SCF₃, CF₃, CN, C₁₋₄-aliphatic residue, C(═O)OC₁₋₄-aliphatic residue and C(═O)OH,
   wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, CN, C₁₋₄-aliphatic residue, C(═O)OH, C(═O)O—C₁₋₄-aliphatic residue, C₃₋₆-cycloaliphatic residue and 3 to 7 membered heterocycloaliphatic residue,
      wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
      wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, ON, C₁₋₄-aliphatic residue and C(═O)OH,
      and
      wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, CF₃, ON, C₁₋₄-aliphatic residue and C(═O)OH,
   on the condition that if R³ denotes a 3 to 10 membered heterocycloaliphatic residue, the 3 to 10 membered heterocycloaliphatic residue is linked via a carbon atom,
   or denotes aryl or heteroaryl, in each case unsubstituted or mono- or polysubstituted with at least one substituent selected from the group consisting of F, Cl, NH₂, an NH(C₁₋₄-aliphatic residue), N(C₁₋₄-aliphatic residue)₂, OH, O—C₁₋₄-aliphatic residue, OCF₃, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

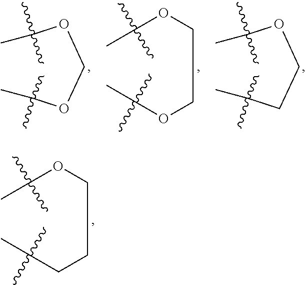 [SMILES: *OCO* ; *OCCO* ; *CCO* ; *CCCO*]

benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, NH₂, NH(C₁₋₄-aliphatic residue), N(C₁₋₄-aliphatic residue)₂, OH, O—C₁₋₄-aliphatic residue, OCF₃, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆ cycloaliphatic residue and the 3 to 7 membered heterocycloaliphatic residue may in each case may be unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, ON, C₁₋₄-aliphatic residue and C(═O)OH,
and wherein the aryl or the heteroaryl residue may in each case be optionally linked, preferably in each case is linked, via a C₁₋₄-aliphatic group, which in turn may be unsubstituted or mono- or polysubstituted with at least one substituent selected from the group consisting of F, Cl, OH, ═O, O—C₁₋₄-aliphatic residue, OCF₃, CF₃, CN and C(═O)OH,
and
R⁴ denotes H or C₁₋₁₀-aliphatic residue, preferably C₁₋₄-aliphatic residue, unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, CN, C₁₋₄-aliphatic residue and C(═O)OH,
   wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue,
or
R³ and R⁴ form together with the nitrogen atom connecting them a 3 to 10 membered heterocyclo-aliphatic residue, preferably a 4 to 7 membered heterocycloaliphatic residue, unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, CN, C₁₋₄-aliphatic residue, C(═O)OH, C₃₋₆-cycloaliphatic residue and 3 to 7 membered heterocycloaliphatic residue,
   wherein the C₁₋₄-aliphatic residue in each case may be unsubstituted or mono- or polysubstituted with at least one substituent selected from the group consisting of F, Cl, OH, ═O, OCF₃, CF₃ and unsubstituted O—C₁₋₄-aliphatic residue, and
   wherein the C₃₋₆ cycloaliphatic residue and the 3 to 7 membered heterocycloaliphatic residue may in each case may be unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, CN, C₁₋₄-aliphatic residue and C(═O)OH,
   and wherein the 3 to 10 membered heterocycloaliphatic residue formed by R³ and R⁴ together with the nitrogen atom connecting them may optionally be condensed with aryl or heteroaryl, preferably with phenyl, pyridyl or thienyl, wherein the aryl or heteroaryl residues fused in this way can for their part be respectively unsubstituted or mono- or polysubstituted with at least one substituent selected from the group consisting of F, Cl, NH₂, NH(C₁₋₄-aliphatic residue), N(C₁₋₄-aliphatic residue)₂, OH, O—C₁₋₄-aliphatic residue, OCF₃, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

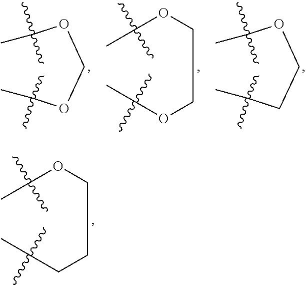 [SMILES: *OCO* ; *OCCO* ; *CCO* ; *CCCO*]

benzyl, phenyl, thienyl, pyridyl, furyl, thiazolyl and oxazolyl,
and wherein the 3 to 10 membered heterocycloaliphatic residue formed by R³ and R⁴ together with the nitrogen atom connecting them may optionally be condensed with a C₃₋₁₀-cycloaliphatic residue or a 3 to 10 membered heterocycloaliphatic residue, wherein the C₃₋₁₀-cycloaliphatic residue or a 3 to 10 membered heterocycloaliphatic residue condensed in this way can for their part be respectively unsubstituted or mono- or polysubstituted with at least one substituent selected from the group consisting of F, Cl, NH₂, NH(C₁₋₄-aliphatic residue), N(C₁₋₄-aliphatic residue)₂, ═O, OH, O—C₁₋₄-aliphatic residue, OCF₃, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, NH₂, NH(C₁₋₄-aliphatic residue), N(C₁₋₄-aliphatic residue)₂, OH, O—C₁₋₄-aliphatic residue, OCF₃, CF₃, CN, C₁₋₄-aliphatic residue, C(═O)OH, C(═O)CH₃, C(═O)C₂H₅, C(═O)OCH₃ and C(═O)OC₂H₅, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, NH₂, NH(C₁₋₄-aliphatic residue), N(C₁₋₄-aliphatic residue)₂, OH, ═O, O—C₁₋₄-aliphatic residue, OCF₃, CF₃, CN, C₁₋₄-aliphatic residue and C(═O)OH.

In a further preferred embodiment of the compound according to general formula (I),
R³ represents the partial structure (T2),

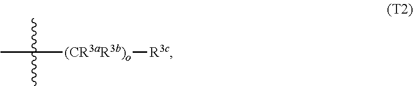

wherein
o denotes 0, 1, 2 or 3,
R^{3a} and R^{3b} each independently of one another represent H, F, Cl, Br, I, O—C₁₋₄-aliphatic residue or C₁₋₄-aliphatic residue or together denote ═O, and
R^{3c} denotes C₁₋₄-aliphatic residue, unsubstituted or mono- or polysubstituted with at least one substituent selected from the group consisting of F, Cl, Br, I, ═O, O—C₁₋₄-aliphatic residue, CF₃ and C₁₋₄-aliphatic residue,
   wherein the C₁₋₄-aliphatic residue in each case may be unsubstituted or mono- or polysubstituted with at least one substituent selected from the group consisting of F, Cl, CF₃ and O—C₁₋₄-aliphatic residue,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
   wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue,
   or denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
   wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, preferably with at least one substituent selected from the group consisting of F, Cl, CH₃, OCH₃, CF₃ and OCF₃, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃, C₁₋₄-aliphatic residue and C(═O)—OH,
and
R⁴ denotes H or unsubstituted C₁₋₄-aliphatic residue or C₁₋₄-aliphatic residue, monosubstituted with OCH₃,
or
R³ and R⁴ form together with the nitrogen atom connecting them a 3 to 10 membered heterocycloaliphatic residue, unsubstituted or mono- or polysubstituted with at least one substituent selected from the group consisting of F, Cl, Br, I, OH, ═O, C(═O)OH, O—C₁₋₄-aliphatic residue, OCF₃, SCF₃, S—C₁₋₄-aliphatic residue, CF₃, C₁₋₄-aliphatic residue, cyclopropyl, cyclobutyl and cyclopentyl,
   wherein the C₁₋₄-aliphatic residue is in each case unsubstituted or mono- or polysubstituted with at least one substituent selected from the group consisting of F, Cl, OH, ═O, CF₃ and O—C₁₋₄-aliphatic residue,
   and wherein the 3 to 10 membered heterocycloaliphatic residue formed by R³ and R⁴ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, O—C₁₋₄-aliphatic residue, OCF₃, SCF₃, S—C₁₋₄-aliphatic residue, CF₃, C₁₋₄-aliphatic residue, C(═O)OH and C₃₋₆-cycloaliphatic residue,
   wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
   wherein the C₃₋₆-cycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, SCF₃, S—C₁₋₄-aliphatic residue, CF₃, C₁₋₄-aliphatic residue and C(═O)OH,
   and wherein the 3 to 10 membered heterocycloaliphatic residue formed by R³ and R⁴ together with the nitrogen atom connecting them may optionally be condensed with a C₃₋₆-cycloaliphatic residue, preferably cyclopropyl, cyclobutyl or cyclopentyl, or a 3 to 7 membered heterocycloaliphatic residue, preferably oxetanyl or oxiranyl, wherein the C₃₋₆-cycloaliphatic residue or the 3 to 7 membered heterocycloaliphatic residue condensed in this way can for their part be respectively unsubstituted or mono- or polysubstituted with at least one substituent selected from the group consisting of F, Cl, Br, I, ═O, OH, O—C₁₋₄-aliphatic residue, OCF₃, SCF₃, CF₃, CN, C₁₋₄-aliphatic residue, C(═O)OH, C(═O)CH₃, C(═O)C₂H₅, C(═O)OCH₃ and C(═O)OC₂H₅.

More preferably,
R³ represents the partial structure (T2),
wherein
o denotes 0, 1, 2 or 3, preferably denotes 1 or 2, more preferably denotes 1,
R^{3a} and R^{3b} each independently of one another represent H, F, Cl, O—C₁₋₄-aliphatic residue or C₁₋₄-aliphatic residue or together denote ═O,
   preferably each independently of one another represent H, F, O—C₁₋₄-aliphatic residue or C₁₋₄-aliphatic residue or together denote ═O, and
R^{3c} denotes C₁₋₄-aliphatic residue, unsubstituted or mono- or polysubstituted with at least one substituent selected from the group consisting of F, Cl, ═O, O—C₁₋₄-aliphatic residue, CF₃, and C₁₋₄-aliphatic residue,
   wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue,
   or denotes C₃₋₁₀-cycloaliphatic residue or 3 to 10 membered heterocycloaliphatic residue, in each case unsubstituted or mono- or polysubstituted with at least one substituent selected from the group consisting of F, Cl, O—C₁₋₄-aliphatic residue, CF₃, and C₁₋₄-aliphatic residue,
   wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue,
   or denotes an aryl or heteroaryl, in each case unsubstituted or mono- or polysubstituted with at least one substituent selected from the group consisting of F, Cl, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₆, C₃₋₆-cycloaliphatic residue, 3 to 7 membered heterocycloaliphatic residue, benzyl, phenyl, thienyl or pyridyl,
      wherein benzyl, phenyl, thienyl and pyridyl, may in each case may be unsubstituted or mono- or polysubstituted, preferably unsubstituted or mono- or disubstituted with at least one substituent selected from the group consisting of F, Cl, OH, O—C₁₋₄-aliphatic residue, OCF₃, CF₃, CN, C₁₋₄-aliphatic residue, C(═O)CH₃, C(═O)C₂H₆, C(═O)OCH₃ and C(═O)OC₂H₆, preferably with at least one substituent selected from the group consisting of F, Cl, CH₃, O—CH₃, CF₃ and OCF₃, and
      wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, OH, ═O, O—C₁₋₄-aliphatic residue, OCF₃, CF₃, C₁₋₄-aliphatic residue and C(═O)OH,
and
R⁴ denotes H or unsubstituted C₁₋₄-aliphatic residue or C₁₋₄-aliphatic residue monosubstituted with OCH₃,
   wherein the C₁₋₄-aliphatic residue is in each case preferably selected from the group consisting of methyl, ethyl, n-propyl, 2-propyl, n-butyl, isobutyl, sec.-butyl and tert.-butyl, more preferably selected from the group consisting of methyl and ethyl,
or
   R³ and R⁴ form together with the nitrogen atom connecting them a 3 to 10 membered heterocycloaliphatic residue, more preferably selected from the group consisting of morpholinyl, piperidinyl, pyrrolidinyl, azetidinyl, piperazinyl, 4-methylpiperazinyl, oxazepanyl, thiomorpholinyl, azepanyl,

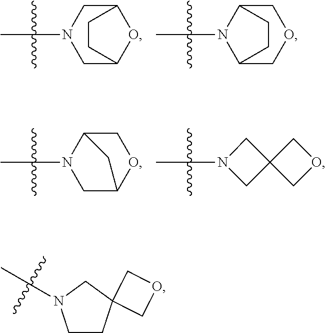 [SMILES: *N1CC2CCC(C1)O2 ; *N1C2CCC1COC2 ; *N1CC2CC1CO2 ; *N1CC2(COC2)C1 ; *N1CCC2(COC2)C1]

in each case unsubstituted or mono- or polysubstituted with at least one substituent selected from the group consisting of F, Cl, OH, ═O, C(═O)OH, O—C₁₋₄-aliphatic residue, OCF₃, CF₃, C₁₋₄-aliphatic residue, cyclopropyl, cyclobutyl and cyclopentyl,
wherein the C₁₋₄-aliphatic residue is in each case unsubstituted or mono- or polysubstituted with at least one substituent selected from the group consisting of F, Cl, OH, ═O, CF₃ and unsubstituted O—C₁₋₄-aliphatic residue, preferably is in each case unsubstituted,
and wherein the 3 to 10 membered heterocycloaliphatic residue formed by R³ and R⁴ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, OH, O—C₁₋₄-aliphatic residue, OCF₃, CF₃, C₁₋₄-aliphatic residue, C(═O)OH and C₃₋₆-cycloaliphatic residue,
wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
wherein the C₃₋₆-cycloaliphatic residue may in each case may be unsubstituted or mono- or polysubstituted with at least one substituent selected from the group consisting of F, Cl, OH, ═O, O—C₁₄ aliphatic residue, OCF₃, CF₃, C₁₋₄-aliphatic residue and C(═O)OH,
and wherein the 3 to 10 membered heterocycloaliphatic residue formed by R³ and R⁴ together with the nitrogen atom connecting them may optionally be condensed with a C₃₋₆-cycloaliphatic residue, preferably cyclopropyl, cyclobutyl or cycclopentyl, or a 4 to 7 membered heterocycloaliphatic residue, preferably oxetanyl or oxiranyl,
wherein the C₃₋₆-cycloaliphatic residue or the 4 to 7 membered heterocycloaliphatic residue condensed in this way can for their part be respectively unsubstituted or mono- or polysubstituted with at least one substituent selected from the group consisting of F, Cl, ═O, OH, O—C₁₋₄-aliphatic residue, OCF₃, CF₃, CN, C₁₋₄-aliphatic residue, C(═O)OH, C(═O)CH₃, C(═O)C₂H₅, C(═O)OCH₃ and C(═O)OC₂H₆.

In a further preferred embodiment of the compound according to general formula (I),
R³ represents the partial structure (T2),
wherein
o denotes 0, 1, 2 or 3,
R^{3a} and R^{3b} each independently of one another represent H, F, CH₃ or OCH₃, or together denote ═O,
R^{3c} denotes C₁₋₄-aliphatic residue, unsubstituted or mono- or polysubstituted with at least one substituent selected from the group consisting of F, Cl, Br, I, ═O, O—C₁₋₄-aliphatic residueand CF₃, or denotes cyclopropyl, cyclobutyl, cyclopentyl or cyclohexyl, pyrrolidinyl, morpholinyl, piperazinyl, piperidinyl or tetrahydropyranyl, in each case unsubstituted or mono- or polysubstituted with at least one substituent selected from the group consisting of F, Cl, Br, I, O—C₁₋₄-aliphatic residue, CF₃ and C₁₋₄-aliphatic residue,
   or denotes aryl or heteroaryl, in each case unsubstituted or mono- or polysubstituted with at least one substituent selected from the group consisting of F, Cl, Br, OH, O—C₁₋₄-aliphatic residue, OCF₃, CF₃, CN, and C₁₋₄-aliphatic residue,
and
R⁴ denotes H, CH₃, CH₂CH₃, CH₂CH₂OCH₃ or CH₂CH₂CH₂OCH₃,
or
R³ and R⁴ form together with the nitrogen atom connecting them a heteroaliphatic residue, selected from the group consisting of morpholinyl, piperidinyl, pyrrolidinyl, azetidinyl, oxazepanyl, tetrahydroquinolinyl, tetrahydroisoquinolinyl, thiomorpholinyl, azepanyl, tetrahydroimidazo[1,2-a]pyrazinyl, octahydropyrrolo[1,2-a]pyrazinyl,

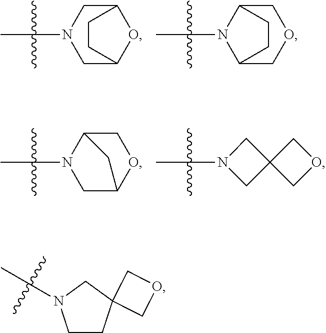 [SMILES: *N1CC2CCC(C1)O2 ; *N1C2CCC1COC2 ; *N1CC2CC1CO2 ; *N1CC2(COC2)C1 ; *N1CCC2(COC2)C1]

dihydroindolinyl, or dihydroisoindolyl, in each case unsubstituted or mono- or polysubstituted with at least one substituent selected from the group consisting of F, Cl, OH, ═O, C(═O)OH, OCH₃, OCH₂CH₃, OCF₃, SCF₃, CF₃, C(═O)CH₃, C(═O)OCH₃, CH₂CF₃, CH₂OH, CH₂OCH₃, CH₂CH₂OCH₃, CH₃, CH₂CH₃, CH₂CH₂CH₃, CH(CH₃)₂ and cyclopropyl.

In a particularly preferred embodiment of the present invention, the compound according to general formula (I) is characterized in that
A¹ represents CR⁵, N;
A² represents CR⁶, N, O, S or NR⁷;
A³ represents CR⁸ or N, and
n denotes 0 or 1,
on the condition, that
   if n denotes 0, then A² represents O, S or NR⁷, or
   if n denotes 1, then A² represents CR⁶ or N,
wherein
   R⁵ denotes F, Cl, CH₃, OCH₃ or CH₂CH₃; and/or
   R⁶ denotes H; and/or
   R⁷ denotes CH₃, CH₂CH₃ or cyclopropyl; and/or
   R⁸ denotes H;
with the proviso, that,
   if n denotes 1, then at least one of A¹, A² and A³ denotes N,
with the proviso, that
   if n denotes 1 and A³ denotes N, then A¹ and/or A² denotes N,
and with the proviso, that
   if n denotes 1 and A² denotes N and A¹ denotes CR⁵ and A³ denotes CR⁸, then R⁵ denotes F, Cl, CH₃, CF₃, CHF₂ or CH₂F;
R¹³ represents H or CH₃;
R¹ represents the partial structure (T1),

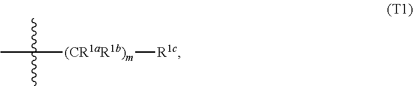

wherein
m denotes 1 or 2,
R^{1a} and R^{1b} represent H and
R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue,
   or
   denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
or wherein
m denotes 0 and
R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)—CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
   wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and
   wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, an O—C₁₋₄-aliphatic residue, OCF₃, CF₃C₁₋₄-aliphatic residue and C(═O)OH;
R² is selected from the group consisting of CH₃, C₂H₅, CH₂CH₂CH₃, CH(CH₃)₂, CH₂CH₂CH₂CH₃, CH₂CH(CH₃)₂, CH(CH₃)CH₂CH₃, C(CH₃)₃, CH₂-cyclopropyl, OCH₃, OC₂H₅, OCH₂CH₂CH₃, OCH(CH₃)₂, O-cyclopropyl, SCH₃, SC₂H₅, SCH₂CH₂CH₃, SCH(CH₃)₂, S-cyclopropyl, cyclopropyl, cyclobutyl, cyclopentyl, cyclohexyl, N(CH₃)₂, N(CH₃)C₂H₅, N(CH₃)CH₂CH₂CH₃, N(CH₃)CH(CH₃)₂, N(CH₃)-cyclopropyl, N(C₂H₅)₂, N(C₂H₅)CH₂CH₂CH₃, N(C₂H₅)CH(CH₃)₂, N(C₂H₅)-cyclopropyl, N-aziridinyl, N-azetidinyl, N-pyrrolidinyl, N-piperidinyl or N-morpholinyl,
   in each case unsubstituted or mono- or polysubstituted with F, OH and/or OCH₃;
R³ represents the partial structure (T2)

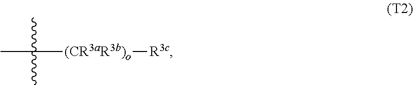

wherein
o denotes 0, 1, 2 or 3,
R^{3a} and R^{3b} each independently of one another represent H, F, CH₃ or OCH₃, or together denote ═O,
R^{3b} denotes C₁₋₄-aliphatic residue, unsubstituted or mono- or polysubstituted with at least one substituent selected from the group consisting of F, Cl, Br, I, ═O, O—C₁₋₄-aliphatic residue and CF₃, or denotes cyclopropyl, cyclobutyl, cyclopentyl or cyclohexyl, pyrrolidinyl, morpholinyl, piperazinyl, piperidinyl or tetrahydropyranyl,
   in each case unsubstituted or mono- or polysubstituted with at least one substituent selected from the group consisting of F, Cl, Br, I, O—C₁₋₄-aliphatic residue, CF₃ and C₁₋₄-aliphatic residue,
   or denotes aryl or heteroaryl, in each case unsubstituted or mono- or polysubstituted with at least one substituent selected from the group consisting of F, Cl, Br, OH, O—C₁₋₄-aliphatic residue, OCF₃, CF₃, CN, and C₁₋₄-aliphatic residue,
and
R⁴ denotes H, CH₃, CH₂CH₃, CH₂CH₂OCH₃ or CH₂CH₂CH₂OCH₃,
or
R³ and R⁴ form together with the nitrogen atom connecting them a morpholinyl, piperidinyl, pyrrolidinyl, azetidinyl, oxazepanyl, tetrahydroquinolinyl, tetrahydroisoquinolinyl, thiomorpholinyl, azepanyl, tetrahydroimidazo[1,2-a]pyrazinyl, octahydropyrrolo[1,2-a]pyrazinyl,

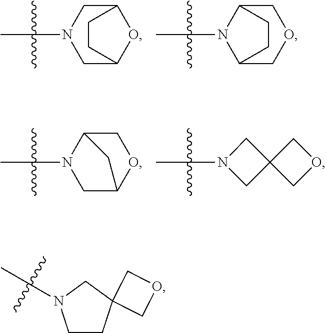 [SMILES: *N1CC2CCC(C1)O2 ; *N1C2CCC1COC2 ; *N1CC2CC1CO2 ; *N1CC2(COC2)C1 ; *N1CCC2(COC2)C1]

dihydroindolinyl, or dihydroisoindolyl, in each case unsubstituted or mono- or polysubstituted with at least one substituent selected from the group consisting of F, Cl, OH, ═O, C(═O)OH, OCH₃, OCH₂CH₃, OCF₃, SCF₃, CF₃, C(═O)CH₃, C(═O)OCH₃, CH₂CF₃, CH₂OH, CH₂OCH₃, CH₂CH₂OCH₃, CH₃, CH₂CH₃, CH₂CH₂CH₃, CH(CH₃)₂ and cyclopropyl.

In another particularly preferred embodiment of the invention, the compound according to general formula (I) is characterized in that
A¹ represents CR⁵, N;
A² represents CR⁶, N, O, S or NR⁷;
A³ represents CR⁸ or N, and
n denotes 0 or 1,
on the condition, that
   if n denotes 0, then A² represents O, S or NR⁷, or
   if n denotes 1, then A² represents CR⁶ or N,
wherein
   R⁵ denotes F, Cl, CH₃, OCH₃ or CH₂CH₃; and/or
   R⁶ denotes H; and/or
   R⁷ denotes CH₃, CH₂CH₃ or cyclopropyl; and/or
   R⁸ denotes H;
with the proviso, that,
   if n denotes 1, then at least one of A¹, A² and A³ denotes N,
with the proviso, that
   if n denotes 1 and A³ denotes N, then A¹ and/or A² denotes N,
and with the proviso, that
   if n denotes 1 and A² denotes N and A¹ denotes CR⁵ and A³ denotes CR⁸, then R⁵ denotes F, Cl, CH₃, CF₃, CHF₂ or CH₂F;
R¹ represents phenyl or pyridyl, preferably phenyl, in each case unsubstituted or mono- or disubstituted with at least one substituent selected from the group consisting of F, Cl, Br, OH, OCH₃, OCF₃, CF₃, and CH₃,
R² denotes CH₂CH₃, CH(CH₃)₂, C(CH₃)₃, cyclopropyl, cyclobutyl or cyclopentyl or tetrahydropyranyl,
   or
   denotes S—R⁹ or O—R¹⁰, wherein R⁹ and R¹⁰ in each case denote CH₃, CH₂CH₃, CH(CH₃)₂ or C(CH₃)₃, or
   denotes N(R¹¹R¹²),
   wherein
   R¹¹ denotes CH₃, CH₂CH₃, CH(CH₃)₂ or C(CH₃)₃,
   R¹² denotes H, CH₃ or CH₂CH₃,
   or
   R¹¹ and R¹² form together with the nitrogen atom connecting them a morpholinyl, piperidinyl, pyrrolidinyl, or azetidinyl, in each case unsubstituted or mono- or polysubstituted with F, OH and/or OCH₃;
R³ denotes cyclopropyl, cyclobutyl, cyclopentyl or cyclohexyl, pyrrolidinyl, morpholinyl, piperazinyl, piperidinyl or tetrahydropyranyl,
   cyclopropylmethyl, cyclobutylmethyl, cyclopentylmethyl or cyclohexylmethyl, pyrrolidin-1-ylmethyl, morpholin-1-ylmethyl, piperazin-1-ylmethyl, piperidin-1-ylmethyl or tetrahydropyran-4-ylmethyl,
      in each case unsubstituted or mono- or polysubstituted with at least one substituent selected from the group consisting of F, C₁₋₁₀—C₁₋₄-aliphatic residue, CF₃ and C₁₋₄-aliphatic residue,
and R⁴ denotes CH₃,
or
R³ and R⁴ form together with the nitrogen atom connecting them heterocycloaliphatic residue, selected from morpholinyl, piperidinyl, pyrrolidinyl, azetidinyl, oxazepanyl, tetrahydroquinolinyl, tetrahydroisoquinolinyl, dihydroindolinyl, or dihydroisoindolyl, in each case unsubstituted or mono- or polysubstituted with F, OH, CH₃ and/or OCH₃.

Especially particularly preferred are compounds according to general formula (I) selected from the group comprising:
1 N-[(4-Chlorophenyl)-methyl]-4-ethylsulfanyl-6-methyl-2-morpholin-4-yl-pyrimidine-5-carboxylic acid amide;
2 N-[(4-Chlorophenyl)-methyl]-4,6-dimethoxy-2-morpholin-4-yl-pyrimidine-5-carboxylic acid amide;
3 N-[(4-Chlorophenyl)-methyl]-4-ethylsulfanyl-2-methyl-6-morpholin-4-yl-pyridine-3-carboxylic acid amide;
4 N-[(4-Chlorophenyl)-methyl]-2-methyl-6-morpholin-4-yl-4-propyl-pyridine-3-carboxylic acid amide;
5 N-(4,4-Dimethyl-pentyl)-4-ethoxy-2-methyl-6-morpholin-4-yl-pyridine-3-carboxylic acid amide;
6 N-(4,4-Dimethyl-pentyl)-4-[(3R)-3-fluoro-pyrrolidin-1-yl]-2-methyl-6-morpholin-4-yl-pyridine-3-carboxylic acid amide;
7 N-[(4-Chlorophenyl)-methyl]-3-isopropyl-5-morpholin-4-yl-pyrazine-2-carboxylic acid amide;
8 N-[(4-Chlorophenyl)-methyl]-3-isopropyl-5-morpholin-4-yl-pyridine-2-carboxylic acid amide;
9 4-Isopropyl-2-(methyl-tetrahydro-pyran-4-yl-amino)-N-[1-[3-(trifluoromethyloxy)-phenyl]-ethyl]-thiazole-5-carboxylic acid amide;
10 4-Ethylsulfanyl-N-[(3-fluorophenyl)-methyl]-6-methyl-2-morpholin-4-yl-pyrimidine-5-carboxylic acid amide;
11 4-Ethylsulfanyl-N-[(3-fluorophenyl)-methyl]-2-methyl-6-morpholin-4-yl-pyridine-3-carboxylic acid amide;
12 4-Ethylsulfanyl-N-[(4-fluorophenyl)-methyl]-2-methyl-6-morpholin-4-yl-pyridine-3-carboxylic acid amide;
13 N-(4,4-Dimethyl-pentyl)-4-ethylsulfanyl-2-methyl-6-morpholin-4-yl-pyridine-3-carboxylic acid amide;
14 N-[(4-Chlorophenyl)-methyl]-4-ethylsulfanyl-2-methyl-6-[(3R)-3-methyl-morpholin-4-yl]-pyridine-3-carboxylic acid amide;
15 N-(4,4-Dimethyl-pentyl)-4-ethylsulfanyl-2-methyl-6-[(3R)-3-methyl-morpholin-4-yl]-pyridine-3-carboxylic acid amide;
16 N-[(4-Chlorophenyl)-methyl]-4-cyclopropyl-2-methyl-6-morpholin-4-yl-pyridine-3-carboxylic acid amide;
17 N-[(4-Chlorophenyl)-methyl]-4-isopropyl-2-methyl-6-morpholin-4-yl-pyridine-3-carboxylic acid amide;
18 N-[(4-Chlorophenyl)-methyl]-4-ethoxy-2-methyl-6-morpholin-4-yl-pyridine-3-carboxylic acid amide;
19 N-[(4-Chlorophenyl)-methyl]-4-dimethylamino-2-methyl-6-morpholin-4-yl-pyridine-3-carboxylic acid amide;
20 N-[(4-Chlorophenyl)-methyl]-3-(1-methyl-propyl)-5-morpholin-4-yl-pyrazine-2-carboxylic acid amide;
21 N-(2-Cyclopentyl-ethyl)-3-(1-methyl-propyl)-5-morpholin-4-yl-pyrazine-2-carboxylic acid amide;
22 4-Isopropyl-2-[methyl-(tetrahydro-pyran-4-yl-methyl)-amino]-N-[1-[3-(trifluoromethyloxy)-phenyl]-ethyl]-thiazole-5-carboxylic acid amide;
23 N-[(4-Chlorophenyl)-methyl]-4-isopropyl-2-(methyl-tetrahydro-pyran-4-yl-amino)-thiazole-5-carboxylic acid amide;
24 N-[(4-Chlorophenyl)-methyl]-4-isopropyl-2-[methyl-(tetrahydro-pyran-4-yl-methyl)-amino]-thiazole-5-carboxylic acid amide;
25 N-[(4-Chlorophenyl)-methyl]-4-isopropyl-2-morpholin-4-yl-thiazole-5-carboxylic acid amide;
26 N-[(4-Chlorophenyl)-methyl]-4-isopropyl-2-piperidin-1-yl-thiazole-5-carboxylic acid amide;
27 N-(4,4-Dimethyl-pentyl)-4-isopropyl-2-piperidin-1-yl-thiazole-5-carboxylic acid amide;
28 N-[(4-Chlorophenyl)-methyl]-4-isopropyl-2-([1,4]oxazepan-4-yl)-thiazole-5-carboxylic acid amide;
29 N-(4,4-Dimethyl-pentyl)-4-isopropyl-2-([1,4]oxazepan-4-yl)-thiazole-5-carboxylic acid amide;
30 N-[(3,4-Difluoro-phenyl)-methyl]-2-morpholin-4-yl-4-propyl-thiazole-5-carboxylic acid amide;
31 N-[(4-Chlorophenyl)-methyl]-4-isopropyl-2-morpholin-4-yl-oxazole-5-carboxylic acid amide;
32 N-(4,4-Dimethyl-pentyl)-4-isopropyl-2-morpholin-4-yl-oxazole-5-carboxylic acid amide;
33 N-(4,4-Dimethyl-pentyl)-5-isopropyl-3-methyl-2-morpholin-4-yl-3H-imidazole-4-carboxylic acid amide;
34 N-[(4-Chlorophenyl)-methyl]-5-isopropyl-3-methyl-2-morpholin-4-yl-3H-imidazole-4-carboxylic acid amide;
35 2-(Methyl-tetrahydro-pyran-4-yl-amino)-4-(trifluoromethyl)-N-[1-[3-(trifluoromethyloxy)-phenyl]-ethyl]-thiazole-5-carboxylic acid amide;
36 2-(Methyl-tetrahydro-pyran-4-yl-amino)-4-(trifluoromethyl)-N-[[3-(trifluoromethyloxy)-phenyl]-methyl]-thiazole-5-carboxylic acid amide;
37 N-[1-(4-Chlorophenyl)-ethyl]-2-(methyl-tetrahydro-pyran-4-yl-amino)-4-(trifluoromethyl)-thiazole-5-carboxylic acid amide;
38 N-[(4-Chlorophenyl)-methyl]-2-(methyl-tetrahydro-pyran-4-yl-amino)-4-(trifluoromethyl)-thiazole-5-carboxylic acid amide;
39 N-[(4-Chlorophenyl)-methyl]-2-[methyl-(tetrahydro-pyran-4-yl-methyl)-amino]-4-(trifluoromethyl)-thiazole-5-carboxylic acid amide;
40 2-Morpholin-4-yl-4-(trifluoromethyl)-N-[1-[3-(trifluoromethyloxy)-phenyl]-ethyl]-thiazole-5-carboxylic acid amide;
2-Morpholin-4-yl-4-(trifluoromethyl)-N-[[3-(trifluoromethyloxy)-phenyl]methyl]-thiazole-5-carboxylic acid amide;
42 N-[1-(4-Chlorophenyl)-ethyl]-2-morpholin-4-yl-4-(trifluoromethyl)-thiazole-5-carboxylic acid amide;
43 N-[(4-Chlorophenyl)-methyl]-2-morpholin-4-yl-4-(trifluoromethyl)-thiazole-5-carboxylic acid amide;
44 2-Morpholin-4-yl-4-(trifluoromethyl)-N-[1-[3-(trifluoromethyl)phenyl]-ethyl]-thiazole-5-carboxylic acid amide;
   respectively in the form of the free compounds, in the form of the salts of physiologically acceptable acids or bases and/or in the form of solvates, and/or, if applicable as racemate; as individual enantiomers, as individual diastereomers, as mixtures of enantiomers in any mixing ratio or as diastereomers in any mixing ratio.

The compounds of the general formula (I) and corresponding stereoisomers and also the respective corresponding salts and solvates are toxicologically safe and are therefore suitable as pharmaceutical active ingredients in pharmaceutical compositions.

In another aspect, the present invention therefore further relates to a pharmaceutical composition containing at least one compound according to general formula (I), in each case if appropriate in the form of one of its pure stereoisomers, in particular enantiomers or diastereomers, its racemates or in the form of a mixture of stereoisomers, in particular the enantiomers and/or diastereomers, in any desired mixing ratio, or respectively in the form of a physiologically acceptable salt, or respectively in the form of a corresponding solvate, and also if appropriate one or more pharmaceutically acceptable auxiliaries.

These pharmaceutical compositions according to the invention are suitable in particular for the modulation of KCNQ2/3 K⁺ channels, preferably for KCNQ2/3 K⁺ channel inhibition and/or KCNQ2/3 K⁺ channel stimulation, i.e. they exert an agonistic or antagonistic effect.

Likewise, the pharmaceutical compositions according to the invention are preferably suitable for the prophylaxis and/or treatment of disorders and/or diseases which are mediated, at least in part, by KCNQ2/3 K⁺ channels. The pharmaceutical composition according to the invention is suitable for administration to adults and children, including toddlers and babies.

The pharmaceutical composition according to the invention may be prepared as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one substituted compound of general formula (I), if appropriate in the form of one of its pure stereoisomers, in particular enantiomers or diastereomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally may contain further physiologically acceptable pharmaceutical auxiliaries which, for example, can be selected from the group consisting of excipients, fillers, solvents, diluents, surface-active substances, dyes, preservatives, blasting agents, slip additives, lubricants, aromas and binders.

The selection of the physiologically acceptable auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragées, capsules, granules, pellets, drops, juices and syrups are preferably suitable for oral application; solutions, suspensions, easily reconstitutable dry preparations and also sprays are preferably suitable for parenteral, topical and inhalative application. The substituted compounds according to the invention used in the pharmaceutical composition according to the invention in a repository, in a dissolved form or in a plaster, and further agents promoting skin penetration being added if appropriate, are suitable percutaneous application preparations. Orally or percutaneously applicable preparation forms can release the respective substituted compound according to the invention also in a delayed manner.

The pharmaceutical compositions according to the invention can be prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula (I) may vary and is for example dependent on the patient's weight or age and also on the type of application, the indication and the severity of the disorder. Conventionally, 0.001 to 100 mg/kg, preferably 0.05 to 75 mg/kg, particularly preferably 0.05 to 50 mg of at least one compound according to the invention are applied per kg of the patient's body weight.

The pharmaceutical composition according to the invention is preferably suitable for the prophylaxis and/or treatment of disorders and/or diseases which are mediated, at least in part, by KCNQ2/3 K⁺ channels. The pharmaceutical composition according to the invention is more preferably suitable for the treatment and/or prophylaxis of one or more diseases and/or disorders selected from the group consisting of pain, in particular pain selected from the group consisting of acute pain, chronic pain, neuropathic pain, muscular pain, visceral pain and inflammatory pain, epilepsy, urinary incontinence, anxiety, dependency, mania, bipolar disorders, migraine, cognitive diseases and dystonia-associated dyskinesias.

The pharmaceutical composition according to the invention is suitable particularly preferably for the treatment of pain, more particularly preferably of acute pain, chronic pain, neuropathic pain, visceral pain, inflammatory pain and muscular pain, and most particularly for the treatment of neuropathic pain.

The pharmaceutical composition according to the invention is also preferably suitable for the treatment and/or prophylaxis of epilepsy.

In a further aspect of the present invention, the invention therefore relates to at least one compound according to general formula (I) and also if appropriate of one or more pharmaceutically acceptable auxiliaries for the prophylaxis and/or treatment of disorders and/or diseases which are mediated, at least in part, by KCNQ2/3 K⁺ channels.

Preference is given to at least one compound according to general formula (I) and optionally one or more pharmaceutically acceptable auxiliaries for the prophylaxis and/or treatment of disorders and/or diseases selected from the group consisting of pain, especially pain selected from the group consisting of acute pain, chronic pain, neuropathic pain, muscular pain, visceral pain and inflammatory pain, epilepsy, urinary incontinence, anxiety, dependency, mania, bipolar disorders, migraine, cognitive diseases and dystonia-associated dyskinesias.

Particular preference is given to at least one compound according to general formula (I) and optionally one or more pharmaceutically acceptable auxiliaries for the prophylaxis and/or treatment of disorders and/or diseases selected from the group consisting of pain, in particular pain selected from the group consisting of acute pain, chronic pain, neuropathic pain, muscular pain, visceral pain and inflammatory pain, most particularly neuropathic pain.

Particular preference is also given to at least one compound according to general formula (I) and optionally one or more pharmaceutically acceptable auxiliaries for the prophylaxis and/or treatment of epilepsy.

In another aspect of the invention, the present invention further relates to at least one compound according to general formula (I) and also if appropriate of one or more pharmaceutically acceptable auxiliaries for use in the preparation of a medicament for prophylaxis and/or treatment of disorders and/or diseases which are mediated, at least in part, by KCNQ2/3 K⁺ channels.

Preference is given to at least one compound according to general formula (I) and optionally one or more pharmaceutically acceptable auxiliaries for use in the preparation of a medicament for the prophylaxis and/or treatment of disorders and/or diseases selected from the group consisting of pain, in particular pain selected from the group consisting of acute pain, chronic pain, neuropathic pain, muscular pain, visceral pain and inflammatory pain, epilepsy, urinary incontinence, anxiety, dependency, mania, bipolar disorders, migraine, cognitive diseases and dystonia-associated dyskinesias.

Particular preference is given to at least one compound according to general formula (I) and optionally one or more pharmaceutically acceptable auxiliaries for use in the preparation of a medicament for the prophylaxis and/or treatment of disorders and/or diseases selected from the group consisting of pain, in particular pain selected from the group consisting of acute pain, chronic pain, neuropathic pain, muscular pain, visceral pain and inflammatory pain, most particularly neuropathic pain.

Particular preference is also given to at least one compound according to general formula (I) and optionally one or more pharmaceutically acceptable auxiliaries for use in the preparation of a medicament for the prophylaxis and/or treatment of epilepsy.

Another aspect of the present invention is a method of treatment and/or prophylaxis of disorders and/or diseases, which are mediated, at least in part, by KCNQ2/3 K⁺ channels, in a mammal, preferably of disorders and/or diseases selected from the group consisting of pain, preferably pain selected from the group consisting of acute pain, chronic pain, neuropathic pain, muscular pain, visceral pain and inflammatory pain, epilepsy, urinary incontinence, anxiety, dependency, mania, bipolar disorders, migraine, cognitive diseases and dystonia-associated dyskinesias, which comprises administering an effective amount of at least one compound of general formula (I) to the mammal.

The effectiveness against pain can be shown, for example, in the Bennett or Chung model (Bennett, G. J. and Xie, Y. K., A peripheral mononeuropathy in rat that produces disorders of pain sensation like those seen in man, Pain 1988, 33(1), 87-107; Kim, S. H. and Chung, J. M., An experimental model for peripheral neuropathy produced by segmental spinal nerve ligation in the rat, Pain 1992, 50(3), 355-363), by tail flick experiments (e.g. according to D'Amour and Smith (J. Pharm. Exp. Ther. 72, 74 79 (1941)) or by the formalin test (e.g. according to D. Dubuisson et al., Pain 1977, 4, 161-174). The effectiveness against epilepsy can be demonstrated, for example, in the DBA/2 mouse model (De Sarro et al., Naunyn-Schmiedeberg's Arch. Pharmacol. 2001, 363, 330-336).

The compounds according to the invention preferably have a EC₅₀ value of not more than 10000 nM or not more than 8000 nM, more preferably not more than 7000 nM or not more than 6000 nM, yet more preferably not more than 5000 nM or not more than 3000 nM, even more preferably not more than 2000 nM or not more than 1000 nM, yet even more preferably not more than 800 nM or not more than 700 nM, still more preferably not more than 600 nM or not more than 500 nM, yet still more preferably not more than 400 nM or not more than 300 nM, most preferably not more than 200 nM or not more than 150 nM and especially not more than 120 nM or not more than 100 nM. Methods for determining the EC₅₀ value are known to the person skilled in the art. The EC₅₀ value is preferably determined by fluorimetry, particularly preferably as described below under “pharmacological experiments”.

The invention further provides processes for the preparation of the substituted compounds according to the invention.

The chemicals and reaction components used in the reactions and schemes described below are available commercially or in each case can be prepared by conventional methods known to the person skilled in the art.

The reactions described can each be carried out under the conventional conditions with which the person skilled in the art is familiar, for example with regard to pressure or the order in which the components are added. If appropriate, the person skilled in the art can determine the optimum procedure under the respective conditions by carrying out simple preliminary tests. The intermediate and end products obtained using the reactions described hereinbefore can each be purified and/or isolated, if desired and/or required, using conventional methods known to the person skilled in the art. Suitable purifying processes are for example extraction processes and chromatographic processes such as column chromatography or preparative chromatography. All of the process steps described below, as well as the respective purification and/or isolation of intermediate or end products, can be carried out partly or completely under an inert gas atmosphere, preferably under a nitrogen atmosphere.

If the compounds according to general formula (I) are obtained, after preparation thereof, in the form of a mixture of their stereoisomers, preferably in the form of their racemates or other mixtures of their various enantiomers and/or diastereomers, they can be separated and if appropriate isolated using conventional processes known to the person skilled in the art. Examples include chromatographic separating processes, in particular liquid chromatography processes under normal pressure or under elevated pressure, preferably MPLC and HPLC processes, and also fractional crystallisation processes. These processes allow individual enantiomers, for example diastereomeric salts formed by means of chiral stationary phase HPLC or by means of crystallisation with chiral acids, for example (+)-tartaric acid, (−)-tartaric acid or (+)-10-camphorsulphonic acid, to be separated from one another.

The various, and in particular the preferred, embodiments of the first aspect of the present invention apply in analogous manner-mutatis mutandis- to the other aspects of the present invention.

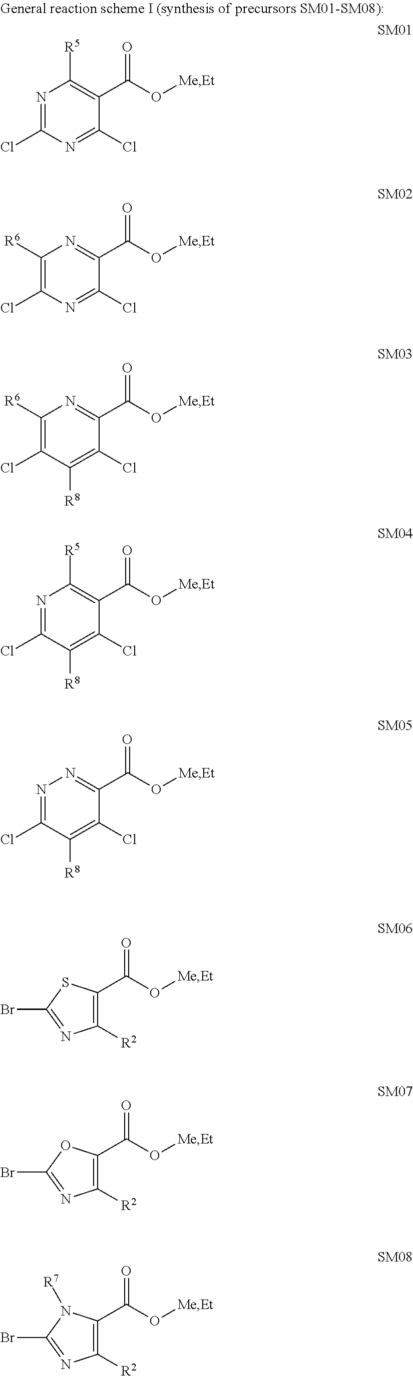

A plurality of syntheses of and synthesis paths to compounds of the general formulae SM01 to SM08 and structurally related precursors with a very broad substitution pattern for residues R⁵, R⁶, R⁷, R⁸ and R² are known in the current specialist literature. Previously unknown intermediates of the general formulae SM01 to SM08 with similar substitution patterns for residues R⁵, R⁶, R⁷, R⁸ and R² as outlined thereafter and whose syntheses are not described in greater detail can be produced by the person skilled in the art according to these known methods or by combination of the known methods.

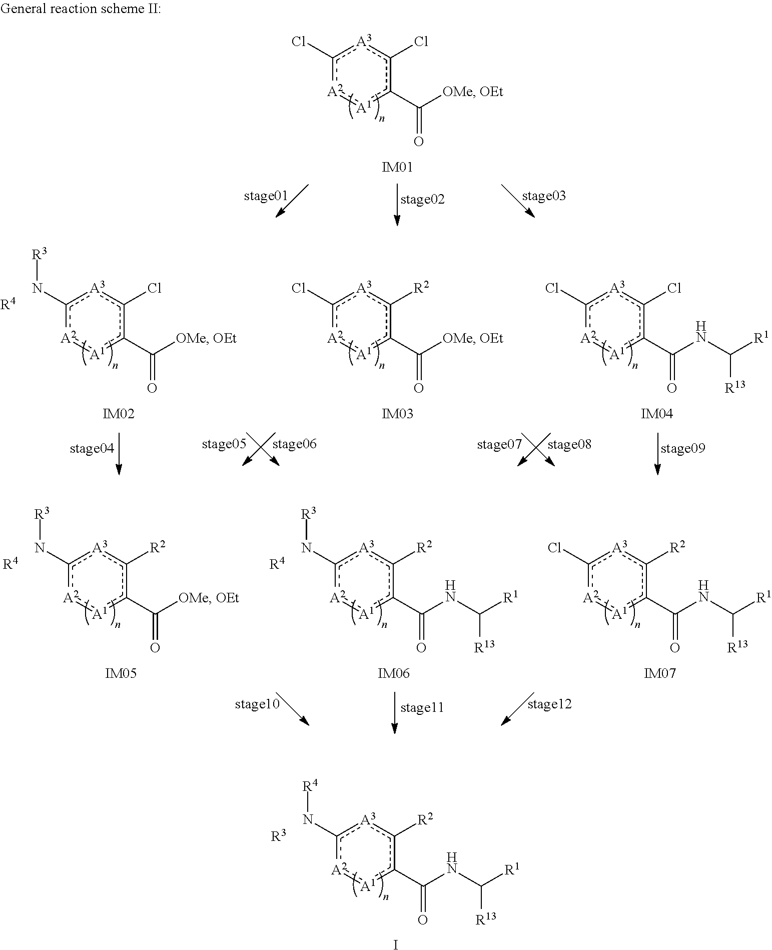

In stage01, stage06, stage08 and stage12, chloro-heteroarenes of the general formulae IM01, IM03, IM04 and IM07 respectively, can be transformed into the corresponding amino-heteroarenes of the general formulae IM02, IM05, IM06 and I respectively, with amines of the general formula HNR²R³ according to methods known to the person skilled in the art, for example by conventional or microwave heating, neat or in solution, for example in acetonitrile, dimethylformamide, dioxane, N-methyl-2-pyrrolidone or tetrahydrofuran, optionally in the presence of a suitable base, for example triethylamine, N,N-diisopropylethylamine, potassium carbonate, caesium carbonate, sodium tert-butoxide or potassium tert-butoxide, optionally by addition of a suitable coupling reagent, for example tetrakis(triphenylphosphin)-palladium, bis(dibenzylideneacetone)-palladium(0), or tris(dibenzylideneacetone)-dipalladium(0), optionally in presence of an additional ligand, for example (2-biphenyl)di-tert-butylphosphine or 2′-bis(diphenylphosphino)-1,1′-binaphthyl.

In stage02, stage04, stage09, and stage11, chloro-heteroarenes of the general formulae IM01, IM02, IM04, and IM06 respectively, can be transformed into the corresponding R²-substituted-heteroarenes of the general formulae IM03, IM05, IM07 and I respectively, with compounds of the general formula Y—R², where Y denotes hydrogen, a metal or organometallic residue, for example sodium, magnesium bromide, magnesium chloride, tributyltin or boronic acid, or a residue to form an organometallic reagent, according to methods known to the person skilled in the art, for example by conventional or microwave heating, neat or in solution, for example in acetonitrile, dimethylformamide, dioxane, N-methyl-2-pyrrolidone, tetrahydrofuran, methanol or ethanol, optionally in the presence of a suitable base, for example triethylamine, N,N-diisopropylethylamine, potassium carbonate, caesium carbonate, sodium tert-butoxide or potassium tert-butoxide, optionally by addition of a suitable coupling reagent, for example tetrakis(triphenylphosphin)-palladium, bis(dibenzylideneacetone)-palladium(0), tris(dibenzylideneacetone)-dipalladium(0), [1,3-bis(diphenylphosphino)propane]-dichloronickel(II) or iron(III) acetylacetonate, optionally in presence of an additional ligand, for example (2-biphenyl)di-tert-butylphosphine or 2′-bis(diphenylphosphino)-1,1′-binaphthyl.

In stage03, stage05, stage07 and stage10, esters of the general formulae IM01, IM02, IM03 and IM05, respectively, can be transformed into amides of the general formulae IM04, IM06, IM07 and I respectively, with amines of the general formula R¹—C(H)(R¹³)—NH₂ according to methods known to the person skilled in the art, for example by the addition of trimethyl aluminium, or by ester hydrolysis to yield the corresponding carboxylic acid followed by reaction with amines of the general formula R¹—C(H)(R¹³)—NH₂ according to methods known to the person skilled in the art, for example using a suitable coupling reagent, for example O-(7-azabenzotriazol-1-yl)-N,N,N′,N′-tetramethyluronium hexafluorophosphate.

Thus obtained compounds of the general formula I can be further transformed to introduce and/or exchange one or more of the substituents R¹, R², R³, R⁴, R⁵, R⁶ and R⁸ by simple derivatization reactions known to the person skilled in the art, for example esterification, ester formation, amide formation, etherification, ether cleavage, oxidation, reduction, hydrogenation, substitution or cross-coupling reactions.

### EXAMPLES

The indication M″ are indications of concentration in mol/I, “MS” means mass spectrometry, “RT” means room temperature (23±7° C.), “TLC” means thin layer chromatography.

### Further Abbreviations:

AcOH acetic acid;
d days
brine saturated aqueous sodium chloride solution
DCM dichloromethane
ether diethyl ether
EtOAc ethyl acetate
EtOH ethanol
h hour(s)
m/z mass-to-charge ratio
MeOH methanol
min minutes
THF tetrahydrofuran
v/v volume in volume
w/w weight in weight

The yields of the compounds prepared were not optimized. All temperatures are uncorrected. All starting materials which are not explicitly described were either commercially available (the details of suppliers such as for example Acros, Avocado, Aldrich, Bachem, Fluke, Lancaster, Maybridge, Merck, Sigma, TCl, Oakwood, etc. can be found in the Symyx® Available Chemicals Database of MDL, San Ramon, US or the SciFinder® Database of the ACS, Washington D.C., US, respectively, for example) or the synthesis thereof has already been described precisely in the specialist literature (experimental guidelines can be found in the Reaxys® Database of Elsevier, Amsterdam, NL or the SciFinder® Database of the ACS, Washington D.C., US, respectively, for example) or can be prepared using the conventional methods known to the person skilled in the art. The mixing ratios of solvents or eluents for chromatography are specified in v/v. All the intermediate products and exemplary compounds were analytically characterised by means of ¹H-NMR spectroscopy. In addition, mass spectrometry tests (MS, m/z for [M+H]⁺) were carried out for all the exemplary compounds and selected intermediate products.

### Synthesis of Exemplary Compounds

### Synthesis of Example 1

### N-[(4-Chlorophenyl)-methyl]-4-ethylsulfanyl-6-methyl-2-morpholin-4-yl-pyrimidine-5-carboxylic acid amide

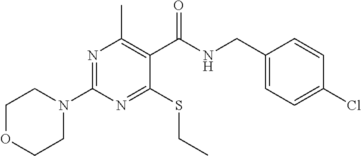 [SMILES: CCSc1nc(N2CCOCC2)nc(C)c1C(=O)NCc1ccc(Cl)cc1]

### a) Synthesis of 3-amino-2-(ethoxycarbonyl-carbamoyl)-but-2-enoic acid methyl ester

To a solution of 3-amino-but-2-enoic acid methyl ester (1.0 g, 8.67 mmol) in dry ether (25 ml) is added N-(oxo-methylene)-carbamic acid ethyl ester (1 g, 8.67 mmol) at 0° C. and the reaction mixture is stirred at 0-5° C. for 4 h. The solid is collected by filtration, rinsed with ether and dried under reduced pressure at RT to get the 3-amino-2-(ethoxycarbonyl-carbamoyl)-but-2-enoic acid methyl ester (0.71 g, 3.08 mmol, 36%).

### b) Synthesis of 4-methyl-2,6-dioxo-3H-pyrimidine-5-carboxylic acid methyl ester

A mixture of 3-amino-2-(ethoxycarbonyl-carbamoyl)-but-2-enoic acid methyl ester (0.71 g, 3.08 mmol) and triethylamine (30% aqueous solution, 0.56 ml) is stirred at 50° C. for 18 h. Another portion of triethylamine (30% aqueous solution, 0.14 ml) is added and the reaction mixture is further at 60° C. for 3 h. The reaction mixture is evaporated and then acidified with AcOH. The resulting solid is collected by filtration and dried under vacuum to get 4-methyl-2,6-dioxo-3H-pyrimidine-5-carboxylic acid methyl ester (0.33 g, 1.79 mmol, 58%), which is directly used for the next step.

### c) Synthesis of 2,4-dichloro-6-methyl-pyrimidine-5-carboxylic acid methyl ester

4-Methyl-2,6-dioxo-3H-pyrimidine-5-carboxylic acid methyl ester (0.64 g, 3.48 mmol) is dissolved in phosphoryl chloride (6.5 ml) and to it is added tributylamine (1.75 ml). The resulting mixture is heated at 95° C. for 3 h. Excess phosphoryl chloride is distilled of and ice-water is added to the reaction mixture and extracted with EtOAc (3×30 ml). The combined organic layers are washed with water (30 ml), brine (30 ml), evaporated to dryness to get the crude product, which is purified by column chromatography (silica gel, 5% EtOAc/hexane) affording 2,4-dichloro-6-methyl-pyrimidine-5-carboxylic acid methyl ester (0.45 g, 2.04 mmol, 59%).

### d) Synthesis of 2-chloro-4-ethylsulfanyl-6-methyl-pyrimidine-5-carboxylic acid methyl ester

To a solution of 2,4-dichloro-6-methyl-pyrimidine-5-carboxylic acid methyl ester (0.18 g, 0.82 mmol) in dimethylformamide (3.5 ml) is added potassium carbonate (0.17 g, 1.23 mmol) at RT, followed by the addition of thioethanol (0.076 ml, 1.03 mmol). The resulting mixture is stirred at RT for 2 h. After completion of reaction, the mixture is diluted with ice water and extracted with EtOAc (3×20 ml). The combined organic layers are washed with brine (20 ml), dried over anhydrous sodium sulfate and evaporated in vacuo to get the crude product, which is purified by column chromatography (silica gel, 5% EtOAc/hexane) to yield 2-chloro-4-ethylsulfanyl-6-methyl-pyrimidine-5-carboxylic acid methyl ester, along with other mono and bis-substituted products. No further attempt is made for purification.

### e) Synthesis of 4-ethylsulfanyl-6-methyl-2-morpholin-4-yl-pyrimidine-5-carboxylic acid methyl ester

The mixture obtained from the previous step (0.16 g, −0.65 mmol) is dissolved in acetonitrile (2.5 ml) and to it are added triethylamine (0.27 ml, 1.95 mmol) and morpholine (114 mg, 1.3 mmol) and stirred at RT for 30 min. After completion of the reaction, the solvent is evaporated and the solid is taken up in ethyl acetate and washed with water. The organic layer is dried over sodium sulfate and evaporated to get the crude which is purified by column chromatography (silica gel, 10% EtOAc/hexane), affording 4-ethylsulfanyl-6-methyl-2-morpholin-4-yl-pyrimidine-5-carboxylic acid methyl ester (0.06 g, 0.2 mmol, 31%).

### f) Synthesis of 4-ethylsulfanyl-6-methyl-2-morpholin-4-yl-pyrimidine-5-carboxylic acid

To a solution of 4-ethylsulfanyl-6-methyl-2-morpholin-4-yl-pyrimidine-5-carboxylic acid methyl ester (0.14 g, 0.47 mmol) in dioxane (3.5 ml) is added 1M aqueous sodium hydroxide (3.5 ml) and heated at 100° C. for 4 h. The reaction mixture is evaporated, diluted with water (10 ml) and acidified with 1M hydrochloric acid to pH 3. The aqueous layer is extracted with EtOAc (5×20 ml) and washed with water (20 ml), brine (20 ml) and dried over sodium sulfate. Evaporation of the solvent affords 4-ethylsulfanyl-6-methyl-2-morpholin-4-yl-pyrimidine-5-carboxylic acid (0.09 g, 0.32 mmol, 68%).

### g) Synthesis of N-[(4-chlorophenyl)-methyl]-4-ethylsulfanyl-6-methyl-2-morpholin-4-yl-pyrimidine-5-carboxylic acid amide

To a stirred solution of 4-ethylsulfanyl-6-methyl-2-morpholin-4-yl-pyrimidine-5-carboxylic acid (0.09 g, 0.32 mmol) in dichloromethane (1.5 ml) are added O-(7-azabenzotriazol-1-yl)-N,N,N′,N′-tetramethyluronium hexafluorophosphate (145 mg, 0.38 mmol) and diisopropylethylamine (0.16 ml, 0.95 mmol) at 0° C. followed by the addition of 4-chlorobenzyl amine (47 mg, 0.38 mmol). The reaction mixture is stirred for 2 h at RT. After completion of the reaction, the mixture is diluted with ice water (10 ml) and extracted with DCM(3×20 ml). The combined organic layers are washed with water (20 ml), brine (20 ml) and dried over anhydrous sodium sulfate. Evaporation under reduced pressure affords the crude product, which is purified by column chromatography (silica gel, 10% acetone/hexane) to yield N-[(4-chlorophenyl)-methyl]-4-ethylsulfanyl-6-methyl-2-morpholin-4-yl-pyrimidine-5-carboxylic acid amide (example 1) (85 mg, 0.21 mmol, 66%). [M+H]⁺407.1.

### Synthesis of Example 2

### N-[(4-Chlorophenyl)-methyl]-4,6-dimethoxy-2-morpholin-4-yl-pyrimidine-5-carboxylic acid amide

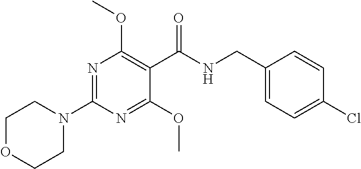 [SMILES: COc1nc(N2CCOCC2)nc(OC)c1C(=O)NCc1ccc(Cl)cc1]

### a) Synthesis of 2-chloro-4,6-dimethoxy-pyrimidine-5-carboxylic acid

2-Chloro-4,6-dimethoxy-pyrimidine (5.0 g, 28.6 mmol) is dissolved in THF (100 ml) and n-butyllithium (15% in hexane) (20.0 ml, 32.0 mmol) is added at −70° C. over a period of 30 min. The mixture is allowed to stir for 30 min at −10° C. before cooling to −70° C. and subsequent addition of solid carbon dioxide (3.78 g, 85.9 mmol). The mixture is stirred for 30 min at −70° C. and then additional 1 h at 0° C. Water is added and the mixture washed with EtOAc (50 ml) and acidified with 2M hydrochloric acid to pH 3. The precipitate is filtered off and dried in a vacuum to yield 2-chloro-4,6-dimethoxy-pyrimidine-5-carboxylic acid (1.85 g, 8.45 mmol, 30%).

### b) Synthesis of 2-chloro-N-[(4-chlorophenyl)-methyl]-4,6-dimethoxy-pyrimidine-5-carboxylic acid amide

To a stirred solution of 2-chloro-4,6-dimethoxy-pyrimidine-5-carboxylic acid (0.66 g, 3.0 mmol) in THF (25 ml) are added O-(7-azabenzotriazol-1-yl)-N,N,N′,N′-tetramethyluronium hexafluorophosphate (1.14 g, 3.0 mmol) and triethylamine (0.16 ml, 0.95 mmol) at followed by the addition of 4-chlorobenzyl amine (0.53 g, 3.75 mmol). The reaction mixture is stirred for 18 h at 55° C. After completion of the reaction, the solvent is distilled off and the solid is taken up in EtOAc (250 ml). The organic layer is washed with water (100 ml), brine (100 ml) and dried over anhydrous sodium sulfate. Evaporation under reduced pressure affords the crude product, which is used without further purification.

### c) Synthesis of N-[(4-chlorophenyl)-methyl]-4,6-dimethoxy-2-morpholin-4-yl-pyrimidine-5-carboxylic acid amide

A mixture of 2-chloro-N-[(4-chlorophenyl)-methyl]-4,6-dimethoxy-pyrimidine-5-carboxylic acid amide (0.51 g, 1.48 mmol), morpholine (0.16 g, 1.85 mmol) and potassium carbonate (0.26 g, 1.85 mmol) in dimethylformamide (6 ml) are heated at 90° C. for 45 min. Water is added and the mixture is extracted with EtOAc (2×30 ml). The combined organic layers are washed with water (100 ml), brine (100 ml) and dried over anhydrous sodium sulfate. Evaporation under reduced pressure affords the crude product, which is crystallized from EtOAc (6 ml) to yield N-[(4-chlorophenyl)-methyl]-4,6-dimethoxy-2-morpholin-4-yl-pyrimidine-5-carboxylic acid amide (example 2) (199 mg, 0.51 mmol, 34%). [M+H]⁺ 393.1.

### Synthesis of Example 3

### N-[(4-Chlorophenyl)-methyl]-4-ethylsulfanyl-2-methyl-6-morpholin-4-yl-pyridine-3-carboxylic acid amide

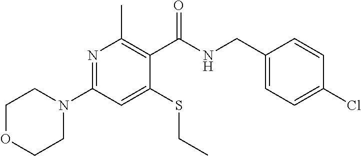 [SMILES: CCSc1cc(N2CCOCC2)nc(C)c1C(=O)NCc1ccc(Cl)cc1]

### a) Synthesis of 4-ethylsulfanyl-2-methyl-6-morpholin-4-yl-pyridine-3-carboxylic acid ethyl ester

To a stirred solution of 4-chloro-2-methyl-6-morpholin-4-yl-pyridine-3-carboxylic acid ethyl ester (1.90 g, 6.69 mmol) in dimethylformamide (30 ml) are added thioethanol (7.5 ml, 100 mmol) and potassium carbonate (2.77 g, 20.1 mmol) at RT. The reaction mixture is heated at 80° C. for 20 h followed by dilution with water (30 ml) and extraction with EtOAc (3×40 ml). The combined organic layers are washed with brine (30 ml), dried over sodium sulfate and evaporated to dryness yielding the crude product, which is purified by column chromatography (silica gel, 15% EtOAc/hexane) to yield 4-ethylsulfanyl-2-methyl-6-morpholin-4-yl-pyridine-3-carboxylic acid ethyl ester (1.6 g, 5.16 mmol, 77%).

### b) Synthesis of 4-ethylsulfanyl-2-methyl-6-morpholin-4-yl-pyridine-3-carboxylic acid

To a solution of 4-ethylsulfanyl-2-methyl-6-morpholin-4-yl-pyridine-3-carboxylic acid ethyl ester (1.14 g, 3.68 mmol) in EtOH (10 ml) are added water (20 ml) and potassium hydroxide (2.06 g, 36.8 mmol) at RT. The reaction mixture is stirred at 100° C. for 8 h. After completion of reaction, the reaction mixture is concentrated and the residue is diluted with water (5 ml). The reaction mixture is washed with EtOAc (15 ml) and the aqueous layer is acidified with 3M hydrochloric acid to pH 3. The aqueous layer is extracted with EtOAc (3×40 ml) and 10% isopropanol/DCM (2×40 ml). The combined organic layers are dried over sodium sulfate and concentrated in vacuo to yield 4-ethylsulfanyl-2-methyl-6-morpholin-4-yl-pyridine-3-carboxylic acid (0.7 g, 2.48 mmol, 68%), which used in to next step without any further purification.

### c) Synthesis of N-[(4-chlorophenyl)-methyl]-4-ethylsulfanyl-2-methyl-6-morpholin-4-yl-pyridine-3-carboxylic acid amide

To a stirred solution of 4-ethylsulfanyl-2-methyl-6-morpholin-4-yl-pyridine-3-carboxylic acid (0.20 g, 0.71 mmol) in dichloromethane (4 ml) are added O-(7-azabenzotriazol-1-yl)-N,N,N′,N′-tetramethyluronium hexafluorophosphate (323 mg, 0.85 mmol) and diisopropylethylamine (0.50 ml, 2.84 mmol) at RT followed by the addition of 4-chlorobenzyl amine (0.12 g, 0.85 mmol). The reaction mixture is stirred for 4 h at RT. After completion of the reaction, the mixture is diluted with ice water (10 ml) and extracted with DCM (3×20 ml). The combined organic layers are washed with water (20 ml), brine (20 ml) and dried over anhydrous sodium sulfate. Evaporation under reduced pressure affords the crude product, which is purified by column chromatography (silica gel, 15% acetone/hexane) to yield N-[(4-chlorophenyl)-methyl]-4-ethylsulfanyl-2-methyl-6-morpholin-4-yl-pyridine-3-carboxylic acid amide (example 3) (0.20 g, 0.49 mmol, 69%). [M+H]⁺406.1.

### Synthesis of Example 4

### N-[(4-Chlorophenyl)-methyl]-2-methyl-6-morpholin-4-yl-4-propyl-pyridine-3-carboxylic acid amide

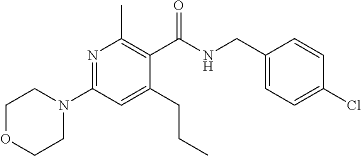 [SMILES: CCCc1cc(N2CCOCC2)nc(C)c1C(=O)NCc1ccc(Cl)cc1]

### a) Synthesis of 2-methyl-6-morpholin-4-yl-4-[(E)-prop-1-enyl]-pyridine-3-carboxylic acid ethyl ester

To a solution of 4-chloro-2-methyl-6-morpholin-4-yl-pyridine-3-carboxylic acid ethyl ester (0.5 g, 1.76 mmol) in toluene (25 ml) are added EtOH (2.5 ml), (1E)-1-propenylboronic acid (0.38 g, 4.40 mmol) and cesium carbonate (2.0 g, 6.16 mmol). The reaction mixture is degassed and flushed with argon for 30 min, followed by the addition of tetrakis(triphenylphosphin)-palladium (223 mg, 0.19 mmol). The reaction mixture is stirred at 120° C. for 24 h and then filtered through a pad of celite and the filtrate is concentrated in vacuo to get the crude product, which is purified by column chromatography (silica gel, 5% EtOAc/hexane) to afford 2-methyl-6-morpholin-4-yl-4-[(E)-prop-1-enyl]-pyridine-3-carboxylic acid ethyl ester (0.17 g, 0.59 mmol, 33%).

### b) Synthesis of 2-methyl-6-morpholin-4-yl-4-propyl-pyridine-3-carboxylic acid ethyl ester

A solution of 2-methyl-6-morpholin-4-yl-4-[(E)-prop-1-enyl]-pyridine-3-carboxylic acid ethyl ester (0.38 g, 1.3 mmol) in MeOH (12 ml) is degassed and flushed with argon for 30 min followed by the addition of 10% palladium on carbon (0.10 g). The reaction mixture is stirred under an atmosphere of hydrogen for 48 h. After completion of reaction, the reaction mixture is filtered through a pad of celite. The filtrate is concentrated in vacuo to get the crude product, which is purified by column chromatography (silica gel, 5% EtOAc/hexane) to afford 2-methyl-6-morpholin-4-yl-4-propyl-pyridine-3-carboxylic acid ethyl ester (0.3 g, 1.03 mmol, 79%).

### c) Synthesis of N-[(4-chlorophenyl)-methyl]-2-methyl-6-morpholin-4-yl-4-propyl-pyridine-3-carboxylic acid amide

To a solution 4-chlorobenzyl amine (0.42 ml, 3.43 mmol) in dry toluene (6 ml) is added trimethylaluminium (2M solution in toluene) (1.7 ml, 3.43 mmol) at 0° C. The reaction mixture is stirred at 0° C. for 1 h followed by the addition of 2-methyl-6-morpholin-4-yl-4-propyl-pyridine-3-carboxylic acid ethyl ester (0.25 g, 0.86 mmol) in dry toluene (4 ml). The reaction mixture is stirred at 120° C. for 16 h. After completion of the reaction, the mixture is poured onto water (15 ml) and extracted with EtOAc (3×15 ml). The combined organic layers are washed with water (15 ml) and brine (15 ml), dried over sodium sulfate and concentrated in vacuo to yield the crude product, which is purified by column chromatography (silica gel, 15% acetone/hexane) to afford N-[(4-chlorophenyl)-methyl]-2-methyl-6-morpholin-4-yl-4-propyl-pyridine-3-carboxylic acid amide (example 4) (0.16 g, 0.41 mmol, 48%). [M+H]⁺388.2.

### Synthesis of Example 5

### N-(4,4-Dimethyl-pentyl)-4-ethoxy-2-methyl-6-morpholin-4-yl-pyridine-3-carboxylic acid amide

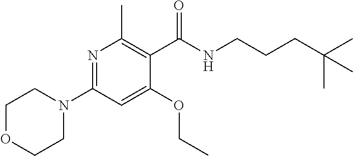 [SMILES: CCOc1cc(N2CCOCC2)nc(C)c1C(=O)NCCCC(C)(C)C]

### a) Synthesis of 4-chloro-2-methyl-6-morpholin-4-yl-pyridine-3-carboxylic acid

A solution of 4-chloro-2-methyl-6-morpholin-4-yl-pyridine-3-carboxylic acid ethyl ester (2.0 g, 7.02 mmol) in EtOH (15 ml) is treated with a solution of lithium hydroxide (0.59 g, 24.6 mmol) in water (15 ml) for 2 h at 80° C. and then for 18 h at RT. The solvent is distilled off, water is added and the mixture is acidified with 2M hydrochloric acid to pH 2 and extracted with EtOAc (4×50 ml). The combined organic layers are dried over magnesium sulphate and evaporated in vacuo to 4-chloro-2-methyl-6-morpholin-4-yl-pyridine-3-carboxylic acid (1.67 g, 6.51 mmol, 93%), which is used in the next step without further purification.

### b) Synthesis of 4-chloro-N-(4,4-dimethyl-pentyl)-2-methyl-6-morpholin-4-yl-pyridine-3-carboxylic acid amide

To a stirred solution of 4-chloro-2-methyl-6-morpholin-4-yl-pyridine-3-carboxylic acid (0.50 g, 1.95 mmol) in THF (10 ml) are added O-(7-azabenzotriazol-1-yl)-N,N,N′,N′-tetramethyluronium hexafluorophosphate (0.74 mg, 1.95 mmol) and triethylamine (1.1 ml, 7.79 mmol) at RT followed by the addition of 4,4-dimethyl-pentan-1-amine (0.33 g, 2.14 mmol). The reaction mixture is stirred for 18 h at RT. After completion of the reaction, the mixture is diluted with 10% ammonium chloride solution (10 ml). The organic layer is washed with saturated sodium hydrogen carbonate (20 ml), brine (20 ml) and dried over anhydrous magnesium sulfate. Evaporation under reduced pressure affords the crude product, which is purified by column chromatography (silica gel, 50% EtOAc/cyclohexane) to yield 4-chloro-N-(4,4-dimethyl-pentyl)-2-methyl-6-morpholin-4-yl-pyridine-3-carboxylic acid amide (0.51 g, 1.44 mmol, 74%).

### c) Synthesis of N-(4,4-dimethyl-pentyl)-4-ethoxy-2-methyl-6-morpholin-4-yl-pyridine-3-carboxylic acid amide

Sodium hydride (60% in mineral oil) (85 mg, 2.12 mmol) is slowly added to EtOH at RT before addition of 4-chloro-N-(4,4-dimethyl-pentyl)-2-methyl-6-morpholin-4-yl-pyridine-3-carboxylic acid amide (0.25 g, 0.71 mmol). The mixture is heated at 100° C. for 6 h. After completion of the reaction, the mixture is poured onto water (15 ml) and extracted with EtOAc (3×15 ml). The combined organic layers are washed with water (15 ml) and brine (15 ml), dried over magnesium sulfate and concentrated in vacuo to yield the crude product, which is purified by column chromatography (silica gel, 50% EtOAc/cyclohexane) to afford N-(4,4-dimethyl-pentyl)-4-ethoxy-2-methyl-6-morpholin-4-yl-pyridine-3-carboxylic acid amide (example 5) (0.18 g, 0.50 mmol, 70%). [M+H]⁺364.3.

### Synthesis of Example 6

### N-(4,4-Dimethyl-pentyl)-4-[(3R)-3-fluoro-pyrrolidin-1-yl]-2-methyl-6-morpholin-4-yl-pyridine-3-carboxylic acid amide

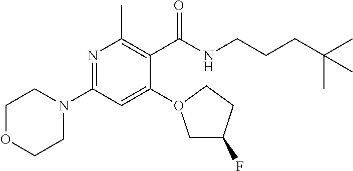

A mixture of 4-chloro-N-(4,4-dimethyl-pentyl)-2-methyl-6-morpholin-4-yl-pyridine-3-carboxylic acid amide (synthesized according to the methods described in sections a) and b) of example 5) (0.27 g, 0.76 mmol), (R)-3-fluoropyrrolidine hydrochloride (0.19 g, 1.53 mmol) and diisopropylethylamine (0.39 ml, 2.29 mmol) in 1-methyl-2-pyrrolidone (2.5 ml) is heated at 160° C. for 4 h. After completion of the reaction, the mixture is diluted with EtOAc and washed with 2M aqueous sodium hydroxide (15 ml), water (2×15 ml) and brine (15 ml), dried over magnesium sulfate and concentrated in vacuo to yield the crude product, which is purified by column chromatography (silica gel, 10% cyclohexane/EtOAc) to afford N-(4,4-dimethyl-pentyl)-4-[(3R)-3-fluoro-pyrrolidin-1-yl]-2-methyl-6-morpholin-4-yl-pyridine-3-carboxylic acid amide (example 6) (0.13 g, 0.32 mmol, 42%). [M+H]⁺407.3.

### Synthesis of Example 7

### N-[(4-Chlorophenyl)-methyl]-3-isopropyl-5-morpholin-4-yl-pyrazine-2-carboxylic acid amide

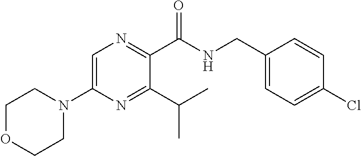 [SMILES: CC(C)c1nc(N2CCOCC2)cnc1C(=O)NCc1ccc(Cl)cc1]

### a) Synthesis of 3,5-dichloro-pyrazine-2-carboxylic acid methyl ester

To a solution of 3,5-dichloro-pyrazine-2-carboxylic acid (0.50 g, 2.60 mmol) in dimethylformamide (8 ml) is added potassium carbonate (0.54 g, 3.90 mmol) at RT followed by the addition of methyl iodide (0.8 ml, 13.0 mmol). The resulting mixture is stirred at RT for 1 h. After completion of the reaction, the mixture is diluted with water (15 ml) and extracted with EtOAc (3×20 ml). The combined organic layers are washed with water (20 ml), brine (20 ml), dried over anhydrous sodium sulfate and evaporated in vacuo to get 3,5-dichloro-pyrazine-2-carboxylic acid methyl ester (0.45 g, 2.18 mmol, 84%), which is used in the next step without further purification.

### b) Synthesis of 3-chloro-5-morpholin-4-yl-pyrazine-2-carboxylic acid methyl ester

To a solution of 3,5-dichloro-pyrazine-2-carboxylic acid methyl ester (1.20 g, 5.82 mmol) in dimethylformamide (12 ml) is added potassium carbonate (0.96 g, 6.98 mmol) at RT followed by the addition of morpholine (0.5 ml, 5.82 mmol). The resulting mixture is stirred at RT for 3 h. After completion of the reaction, the mixture is diluted with water (20 ml) and extracted with EtOAc (3×30 ml). The combined organic layers are washed with water (30 ml), brine (30 ml), dried over anhydrous sodium sulfate and evaporated in vacuo to get the crude product, which is purified by column chromatography (silica gel, 10% acetone/hexane) to yield 3-chloro-5-morpholin-4-yl-pyrazine-2-carboxylic acid methyl ester (1.10 g, 4.28 mmol, 73%).

### c) Synthesis of 3-isopropenyl-5-morpholin-4-yl-pyrazine-2-carboxylic acid methyl ester

To a solution of 3-chloro-5-morpholin-4-yl-pyrazine-2-carboxylic acid methyl ester (0.50 g, 1.94 mmol) in 1,4-dioxane (10 ml) is added tributyl-isopropenyl stannane (1.37 g, 4.13 mmol) at RT. The resulting mixture is degassed and flushed with argon for 30 min followed by the addition of bis(triphenylphosphine)palladiumchloride (0.20 g, 0.29 mmol). The reaction mixture is heated at 110° C. for 16 h. After completion of the reaction, the solvent is distilled off and the crude product is purified by column chromatography (5% potassium fluoride/silica gel, 30% acetone/hexane) to yield 3-isopropenyl-5-morpholin-4-yl-pyrazine-2-carboxylic acid methyl ester (0.32 g, 1.22 mmol, 62%).

### d) Synthesis of 3-isopropyl-5-morpholin-4-yl-pyrazine-2-carboxylic acid methyl ester

To a solution of 3-isopropenyl-5-morpholin-4-yl-pyrazine-2-carboxylic acid methyl ester (0.32 g, 1.22 mmol) in EtOAc (8 ml) is added 20% palladium(II) hydroxide (0.10 g) at RT. The resulting mixture is stirred at RT for 16 h under an atmosphere of hydrogen. After completion of the reaction, the catalyst is filtered off and washed with EtOAc. The filtrate is concentrated to yield 3-isopropyl-5-morpholin-4-yl-pyrazine-2-carboxylic acid methyl ester (0.31 g, 1.17 mmol, 95%), which is used in the next step without further purification.

### e) Synthesis of N-[(4-Chlorophenyl)-methyl]-3-isopropyl-5-morpholin-4-yl-pyrazine-2-carboxylic acid amide

To a solution 4-chlorobenzyl amine (0.57 ml, 4.68 mmol) in dry toluene (5 ml) is added trimethylaluminium (2M solution in toluene) (2.3 ml, 4.68 mmol) at 0° C. The reaction mixture is stirred at 0° C. for 45 min followed by the addition of 3-isopropyl-5-morpholin-4-yl-pyrazine-2-carboxylic acid methyl ester (0.31 g, 1.17 mmol) in dry toluene (3 ml). The reaction mixture is stirred at 110° C. for 1.5 h. After completion of the reaction, the mixture is poured onto saturated ammonium chloride solution (15 ml) and extracted with EtOAc (3×15 ml). The combined organic layers are washed with water (15 ml) and brine (15 ml), dried over sodium sulfate and concentrated in vacuo to yield the crude product, which is purified by column chromatography (silica gel, 15% acetone/hexane) to afford N-[(4-Chlorophenyl)-methyl]-3-isopropyl-5-morpholin-4-yl-pyrazine-2-carboxylic acid amide (example 7) (0.27 g, 0.72 mmol, 61%). [M+H]⁺375.2.

### Synthesis of Example 8

### N-[(4-Chlorophenyl)-methyl]-3-isopropyl-5-morpholin-4-yl-pyridine-2-carboxylic acid amide

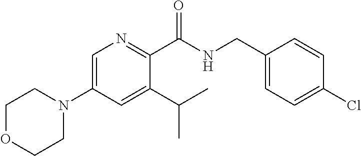 [SMILES: CC(C)c1cc(N2CCOCC2)cnc1C(=O)NCc1ccc(Cl)cc1]

### a) Synthesis of 5-morpholin-4-yl-3-nitro-pyridine-2-carbonitrile

5-Bromo-3-nitro-pyridine-2-carbonitrile (4.0 g, 17.5 mmol, 1 eq.) is dissolved in dimethylsulfoxid (25 ml) and morpholine (3.79 ml, 43.6 mmol) is added. The reaction mixture is stirred at RT for 5 h. After completion of reaction, the mixture is poured onto water (50 ml), extracted with EtOAc (2×75 ml) and the combined organic layers are dried over sodium sulfate, concentrated in vacuo and purified by column chromatography (silica gel, 25% EtOAc/hexane) affording 5-morpholin-4-yl-3-nitro-pyridine-2-carbonitrile (2.8 g, 11.9 mmol, 68%).

### b) Synthesis of 3-methoxy-5-morpholin-4-yl-pyridine-2-carbonitrile

5-Morpholin-4-yl-3-nitro-pyridine-2-carbonitrile (2.8 g, 11.9 mmol) is dissolved in methanol (40 ml) and a solution of solution of sodium methoxide (0.97 g, 17.9 mmol) in MeOH (20 ml) is added. The reaction mixture is heated at 75° C. for 16 h. MeOH is distilled off and the crude product is taken up in water and extracted with EtOAc (3×40 ml). The combined organic layers are dried over sodium sulfate, concentrated in vacuo to afford 3-methoxy-5-morpholin-4-yl-pyridine-2-carbonitrile (2.1 g, 9.58 mmol, 83%).

### c) Synthesis of 3-hydroxy-5-morpholin-4-yl-pyridine-2-carbonitrile

3-Methoxy-5-morpholin-4-yl-pyridine-2-carbonitrile (2.5 g, 11.41 mmol, 1 eq.) is dissolved in dimethylformamide (40 ml) and thioethanol (6.8 ml, 91.3 mmol) and potassium carbonate (6.3 g, 45.6 mmol) are added subsequently. The resulting mixture is heated at 90° C. for 16 h. The reaction mixture is diluted with water (40 ml) and the solution is acidified with 2M hydrochloric acid at 0° C. The solid precipitate thus obtained is filtered and dried. The crude product (2.7 g) is used in the next step without further purification.

### d) Synthesis of trifluoro-methanesulfonic acid (2-cyano-5-morpholin-4-yl-pyridin-3-yl) ester

To a solution of 3-hydroxy-5-morpholin-4-yl-pyridine-2-carbonitrile (2.7 g, ˜13.1 mmol) in DCM (30 ml) are added pyridine (1.5 ml, 19.7 mmol), trifluoromethanesulfonic anhydride (2.9 ml, 19.7 mmol) at 0° C. subsequently. The reaction mixture is warmed to RT and stirred for 3 h. The mixture is poured onto water and extracted with DCM (3×30 ml). The combined organic layers are washed with brine (30 ml), dried over sodium sulfate and concentrated in vacuo to get the crude product, which is purified by column chromatography (silica gel, 30% EtOAc/hexane) affording trifluoro-methanesulfonic acid (2-cyano-5-morpholin-4-yl-pyridin-3-yl) ester (1.8 g, 5.35 mmol, 47%, 2 steps).

### e) Synthesis of 3-isopropenyl-5-morpholin-4-yl-pyridine-2-carbonitrile

To a solution of trifluoro-methanesulfonic acid (2-cyano-5-morpholin-4-yl-pyridin-3-yl) ester (1.0 g, 2.96 mmol) in 1-methyl-2-pyrrolidone (20 ml) are added lithium chloride (0.38 g, 8.88 mmol), triphenylarsine (72 mg, 0.24 mmol) and the mixture is degassed and flushed with argon for 30 min before the addition of tris(dibenzylideneacetone)-dipalladium(0) (62 mg, 0.03 mmol) and copper(I) iodide (28 mg, 0.15 mmol). The mixture is stirred at RT 10 min and tributyl-isopropenyl stannane (1.28 g, 3.55 mmol) is added. The reaction mixture is heated at 120° C. for 16 h. After completion of the reaction, saturated potassium fluoride solution (50 ml) is added and stirring is continued for 30 min. The mixture is extracted with EtOAc (3×40 ml) and the combined organic layers are washed with brine (40 ml) and water (40 ml), dried over sodium sulfate and evaporated to dryness to get the crude product, which is purified by column chromatography (10% potassium fluoride/silica gel, 50% EtOAc/hexane) affording 3-isopropenyl-5-morpholin-4-yl-pyridine-2-carbonitrile (0.37 g, 1.61 mmol, 54%).

### f) Synthesis of 3-isopropyl-5-morpholin-4-yl-pyridine-2-carbonitrile

3-Isopropenyl-5-morpholin-4-yl-pyridine-2-carbonitrile (0.37 g, 1.61 mmol) is dissolved in EtOH (15 ml) and 10% palladium on carbon (85 mg) is added. The mixture is stirred under an atmosphere of hydrogen for 2 h. After completion of the reaction, the mixture is filtered through a pad of celite and concentrated in vacuo yielding 3-isopropyl-5-morpholin-4-yl-pyridine-2-carbonitrile (0.36 g, 1.60 mmol, 54%), which is used in the next step without further purification.

### g) Synthesis of 3-isopropyl-5-morpholin-4-yl-pyridine-2-carboxylic acid

To a solution of yielding 3-isopropyl-5-morpholin-4-yl-pyridine-2-carbonitrile (0.36 g, 1.56 mmol) in EtOH (6 ml) is added a 4M aqueous solution of sodium hydroxide (6 ml). The solution is herated at 110° C. for 16 h and then filtered. The filtrate is concentrated and acidified with 2M hydrochloric acid to pH 3. The aqueous layer is extracted with 10% MeOH/DCM, dried over sodium sulfate and evaporated in vacuo affording 3-isopropyl-5-morpholin-4-yl-pyridine-2-carboxylic acid (0.27 g, 1.08 mmol, 69%).

### h) Synthesis of N-[(4-chlorophenyl)-methyl]-3-isopropyl-5-morpholin-4-yl-pyridine-2-carboxylic acid amide

To a stirred solution of 3-isopropyl-5-morpholin-4-yl-pyridine-2-carboxylic acid (0.10 g, 0.39 mmol) in DCM (5 ml) are added O-(7-azabenzotriazol-1-yl)-N,N,N′,N′-tetramethyluronium hexafluorophosphate (176 mg, 0.47 mmol) and diisopropylethylamine (0.27 ml, 1.55 mmol) at 0° C. followed by the addition of 4-chlorobenzyl amine (0.14 ml, 0.47 mmol). The reaction mixture is stirred for 2 h at RT. After completion of the reaction, the mixture is diluted with ice water (10 ml) and extracted with DCM (3×20 ml). The combined organic layers are washed with water (20 ml), brine (20 ml) and dried over anhydrous sodium sulfate. Evaporation under reduced pressure affords the crude product, which is purified by column chromatography (silica gel, 10% acetone/hexane) to yield N-[(4-chlorophenyl)-methyl]-3-isopropyl-5-morpholin-4-yl-pyridine-2-carboxylic acid amide (example 8) (0.07 g, 0.19 mmol, 50%). [M+H]⁺374.2.

### Synthesis of Example 9

### 4-Isopropyl-2-(methyl-tetrahydro-pyran-4-yl-amino)-N-[1-[3-(trifluoromethyloxy)-phenyl]ethyl]-thiazole-5-carboxylic acid amide

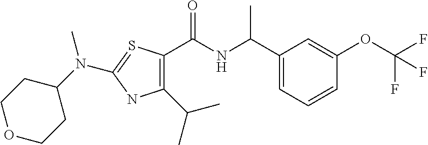

### a) Synthesis of 2-bromo-4-methyl-3-oxo-pentanoic acid ethyl ester

To a stirred mixture of 4-methyl-3-oxo-pentanoic acid ethyl ester (5.0 g, 31.5 mmol) in carbon tetrachloride/water (1:1) (30 ml) is added a solution of bromine (1.61 ml, 31.5 mmol) in carbon tetrachloride (15 ml) at 0° C. over a period of 1 h and the resulting reaction mixture is stirred at 0° C. for 1 h. After completion of the reaction, the organic layer is washed with water (100 ml), brine (50 ml), dried over sodium sulfate and evaporated in vacuo to yield 2-bromo-4-methyl-3-oxo-pentanoic acid ethyl ester (6.7 g, 28.37 mmol, 90%).

### b) Synthesis of 2-amino-4-isopropyl-thiazole-5-carboxylic acid ethyl ester

To a solution of 2-bromo-4-methyl-3-oxo-pentanoic acid ethyl ester (6.7 g, 28.4 mmol) in EtOH (25 ml) is added thiourea (2.15 g, 28.4 mmol) at RT and the reaction mixture is refluxed at 100° C. for 1 h. After completion of the reaction, the mixture is poured onto ice water (100 ml) and neutralized with 18M ammonia solution and extracted with EtOAc (3×100 ml). The combined organic layers are washed with water (100 ml), brine (50 ml), dried over sodium sulfate and concentrated in vacuo. The residue obtained is purified by column chromatography (silica gel, 20% EtOAc/hexane) affording 2-amino-4-isopropyl-thiazole-5-carboxylic acid ethyl ester (4.7 g, 21.9 mmol, 77%).

### c) Synthesis of 2-bromo-4-isopropyl-thiazole-5-carboxylic acid ethyl ester

A slurry of 2-amino-4-isopropyl-thiazole-5-carboxylic acid ethyl ester (4.65 g, 21.7 mmol) and sodium bromide (4.46 g, 43.4 mmol) in water (30 ml) is added to a stirred solution of copper(II) sulfate pentahydrate (6.50 g, 26.0 mmol) and sulfuric acid (70 ml) at −10° C. A solution of sodium nitrite (3.29 g, 47.7) in water (10 ml) is added to the reaction mixture over a period of 30 min and the reaction mixture is warmed to 10° C. over a period of 1 h. After completion of the reaction, the mixture is extracted with ether (200 ml). The organic layer is washed with water (100 ml), brine (50 ml), dried over sodium sulfate and evaporated to yield 2-bromo-4-isopropyl-thiazole-5-carboxylic acid ethyl ester (3.5 g, 12.6 mmol, 58%).

### d) Synthesis of 4-isopropyl-2-(methyl-tetrahydro-pyran-4-yl-amino)-thiazole-5-carboxylic acid ethyl ester

To a solution of 2-bromo-4-isopropyl-thiazole-5-carboxylic acid ethyl ester (0.50 g, 1.80 mmol) in 1-methyl-2-pyrrolidone (10 ml) are added potassium carbonate (0.37 g, 2.70 mmol) and N-methyltetrahydro-2H-pyran-4-amine (0.62 g, 5.40 mmol) at RT and the reaction mixture is heated at 150° C. for 16 h. After completion of the reaction, the mixture is diluted with methyl tert-butyl ether (20 ml) and washed with water (3×20 ml) and brine (3×20 ml). The organic layer is dried over anhydrous sodium sulfate and evaporated to get the crude product, which is purified by column chromatography (silica gel, 10% EtOAc/hexane) to yield 4-isopropyl-2-(methyl-tetrahydro-pyran-4-yl-amino)-thiazole-5-carboxylic acid ethyl ester (0.56 g, 1.92 mmol, 99%).

### e) Synthesis of 4-isopropyl-2-(methyl-tetrahydro-pyran-4-yl-amino)-thiazole-5-carboxylic acid

To a solution of 4-isopropyl-2-(methyl-tetrahydro-pyran-4-yl-amino)-thiazole-5-carboxylic acid ethyl ester (0.60 g, 1.92 mmol) in EtOH (1.5 ml) is added a solution of potassium hydroxide (0.27 g, 4.81 mmol) in water (1 ml) at RT and the reaction mixture is refluxed at 120° C. for 16 h. After completion of the reaction, the solvent is evaporated and the residue is diluted with water (10 ml) and acidified with 2M hydrochloric acid to pH 3. The aqueous layer is extracted with EtOAc (3×15 ml) followed by 10% isopropanol/chloroform (2×15 ml). The combined organic layers are washed with water (20 ml), brine (20 ml), dried over sodium sulfate and concentrated in vacuo. The crude product is washed with 10% ether/pentane to yield 4-isopropyl-2-(methyl-tetrahydro-pyran-4-yl-amino)-thiazole-5-carboxylic acid (0.40 g, 1.408 mmol, 73%).

### f) Synthesis of 4-isopropyl-2-(methyl-tetrahydro-pyran-4-yl-amino)-N-[1-[3-(trifluoromethyloxy)-phenyl]-ethyl]-thiazole-5-carboxylic acid amide

To a stirred solution of 4-isopropyl-2-(methyl-tetrahydro-pyran-4-yl-amino)-thiazole-5-carboxylic acid (0.15 g, 0.53 mmol) in dimethylformamide (3 ml) are added N-methylmorpholine (0.12 ml, 1.06 mmol), O-(benzotriazol-1-yl)-N,N,N′,N′-tetramethyluronium tetrafluoroborate (0.25 g, 0.79 mmol) at 0° C. The reaction mixture is stirred at 0° C. for 5 min before 1-[3-(trifluoromethyloxy)-phenyl]-ethyl-amine (0.13 g, 0.63 mmol) is added. The reaction mixture is then stirred at RT for 16 h. After completion of the reaction, the reaction mixture is diluted with EtOAc (10 ml). The organic layer is washed with saturated sodium hydrogen carbonate solution (15 ml), saturated ammonium chloride solution (15 ml), water (20 ml), and brine (20 ml), dried over sodium sulfate and evaporated to dryness to get the crude product, which is purified by column chromatography (silica gel, 5% acetone/hexane) followed by crystallization from acetone/pentane affording 4-isopropyl-2-(methyl-tetrahydro-pyran-4-yl-amino)-N-[1-[3-(trifluoromethyloxy)-phenyl]-ethyl]-thiazole-5-carboxylic acid amide (example 9) (0.15 g, 0.31 mmol, 58%). [M+H]⁺ 472.2.

### Synthesis of Further Examples

The synthesis of further examples was carried out according to the methods already described. Table 1 shows which compound were produced according to which method. It is evident to the person skilled in the art which educts and reagents were used in each case.

TABLE 1

| | | Preparation | |
|---|---|---|---|
| | | according to | MS m/z |
| Example | Chemical name | example | [M + H]⁺ |
| 10 | 4-Ethylsulfanyl-N-[(3-fluorophenyl)-methyl]-6-methyl-2- | 1 | 391.2 |
| | morpholin-4-yl-pyrimidine-5-carboxylic acid amide | | |
| 11 | 4-Ethylsulfanyl-N-[(3-fluorophenyl)-methyl]-2-methyl-6- | 3 | 390.2 |
| | morpholin-4-yl-pyridine-3-carboxylic acid amide | | |
| 12 | 4-Ethylsulfanyl-N-[(4-fluorophenyl)-methyl]-2-methyl-6- | 3 | 390.2 |
| | morpholin-4-yl-pyridine-3-carboxylic acid amide | | |
| 13 | N-(4,4-Dimethyl-pentyl)-4-ethylsulfanyl-2-methyl-6- | 3 | 380.4 |
| | morpholin-4-yl-pyridine-3-carboxylic acid amide | | |
| 14 | N-[(4-Chlorophenyl)-methyl]-4-ethylsulfanyl-2-methyl- | 3 | 420.2 |
| | 6-[(3R)-3-methyl-morpholin-4-yl]-pyridine-3-carboxylic | | |
| | acid amide | | |
| 15 | N-(4,4-Dimethyl-pentyl)-4-ethylsulfanyl-2-methyl-6- | 3 | 394.3 |
| | [(3R)-3-methyl-morpholin-4-yl]-pyridine-3-carboxylic | | |
| | acid amide | | |
| 16 | N-[(4-Chlorophenyl)-methyl]-4-cyclopropyl-2-methyl-6- | 4 | 386.2 |
| | morpholin-4-yl-pyridine-3-carboxylic acid amide | | |
| 17 | N-[(4-Chlorophenyl)-methyl]-4-isopropyl-2-methyl-6- | 4 | 388.2 |
| | morpholin-4-yl-pyridine-3-carboxylic acid amide | | |
| 18 | N-[(4-Chlorophenyl)-methyl]-4-ethoxy-2-methyl-6- | 5 | 390.2 |
| | morpholin-4-yl-pyridine-3-carboxylic acid amide | | |
| 19 | N-[(4-Chlorophenyl)-methyl]-4-dimethylamino-2- | 6 | 389.2 |
| | methyl-6-morpholin-4-yl-pyridine-3-carboxylic acid | | |
| | amide | | |
| 20 | N-[(4-Chlorophenyl)-methyl]-3-(1-methyl-propyl)-5- | 7 | 389.2 |
| | morpholin-4-yl-pyrazine-2-carboxylic acid amide | | |
| 21 | N-(2-Cyclopentyl-ethyl)-3-(1-methyl-propyl)-5- | 7 | 361.3 |
| | morpholin-4-yl-pyrazine-2-carboxylic acid amide | | |
| 22 | 4-Isopropyl-2-[methyl-(tetrahydro-pyran-4-yl-methyl)- | 9 | 486.2 |
| | amino]-N-[1-[3-(trifluoromethyloxy)-phenyl]-ethyl]- | | |
| | thiazole-5-carboxylic acid amide | | |
| 23 | N-[(4-Chlorophenyl)-methyl]-4-isopropyl-2-(methyl- | 9 | 408.2 |
| | tetrahydro-pyran-4-yl-amino)-thiazole-5-carboxylic acid | | |
| | amide | | |
| 24 | N-[(4-Chlorophenyl)-methyl]-4-isopropyl-2-[methyl- | 9 | 422.2 |
| | (tetrahydro-pyran-4-yl-methyl)-amino]thiazole-5- | | |
| | carboxylic acid amide | | |
| 25 | N-[(4-Chlorophenyl)-methyl]-4-isopropyl-2-morpholin-4- | 9 | 380.1 |
| | yl-thiazole-5-carboxylic acid amide | | |
| 26 | N-[(4-Chlorophenyl)-methyl]-4-isopropyl-2-piperidin-1- | 9 | 378.1 |
| | yl-thiazole-5-carboxylic acid amide | | |
| 27 | N-(4,4-Dimethyl-pentyl)-4-isopropyl-2-piperidin-1-yl- | 9 | 352.2 |
| | thiazole-5-carboxylic acid amide | | |
| 28 | N-[(4-Chlorophenyl)-methyl]-4-isopropyl-2- | 9 | 394.2 |
| | ([1,4]oxazepan-4-yl)-thiazole-5-carboxylic acid amide | | |
| 29 | N-(4,4-Dimethyl-pentyl)-4-isopropyl-2-([1,4]oxazepan- | 9 | 378.1 |
| | 4-yl)-thiazole-5-carboxylic acid amide | | |
| 30 | N-[(3,4-Difluoro-phenyl)-methyl]-2-morpholin-4-yl-4- | 9 | 382.1 |
| | propyl-thiazole-5-carboxylic acid amide | | |
| 31 | N-[(4-Chlorophenyl)-methyl]-4-isopropyl-2-morpholin-4- | 9 | 364.2 |
| | yl-oxazole-5-carboxylic acid amide | | |
| 32 | N-(4,4-Dimethyl-pentyl)-4-isopropyl-2-morpholin-4-yl- | 9 | 338.2 |
| | oxazole-5-carboxylic acid amide | | |
| 33 | N-(4,4-Dimethyl-pentyl)-5-isopropyl-3-methyl-2- | 9 | 351.3 |
| | morpholin-4-yl-3H-imidazole-4-carboxylic acid amide | | |
| 34 | N-[(4-Chlorophenyl)-methyl]-5-isopropyl-3-methyl-2- | 9 | 377.1 |
| | morpholin-4-yl-3H-imidazole-4-carboxylic acid amide | | |
| 35 | 2-(Methyl-tetrahydro-pyran-4-yl-amino)-4- | 9 | 498.1 |
| | (trifluoromethyl)-N-[1-[3-(trifluoromethyloxy)-phenyl]- | | |
| | ethyl]-thiazole-5-carboxylic acid amide | | |
| 36 | 2-(Methyl-tetrahydro-pyran-4-yl-amino)-4- | 9 | 484.1 |
| | (trifluoromethyl)-N-[[3-(trifluoromethyloxy)-phenyl]- | | |
| | methyl]-thiazole-5-carboxylic acid amide | | |
| 37 | N-[1-(4-Chlorophenyl)-ethyl]-2-(methyl-tetrahydro- | 9 | 448.1 |
| | pyran-4-yl-amino)-4-(trifluoromethyl)-thiazole-5- | | |
| | carboxylic acid amide | | |
| 38 | N-[(4-Chlorophenyl)-methyl]-2-(methyl-tetrahydro- | 9 | 434.1 |
| | pyran-4-yl-amino)-4-(trifluoromethyl)-thiazole-5- | | |
| | carboxylic acid amide | | |
| 39 | N-[(4-Chlorophenyl)-methyl]-2-[methyl-(tetrahydro- | 9 | 448.1 |
| | pyran-4-yl-methyl)-amino]-4-(trifluoromethyl)-thiazole- | | |
| | 5-carboxylic acid amide | | |
| 40 | 2-Morpholin-4-yl-4-(trifluoromethyl)-N-[1-[3- | 9 | 470.1 |
| | (trifluoromethyloxy)-phenyl]ethyl]-thiazole-5-carboxylic | | |
| | acid amide | | |
| 41 | 2-Morpholin-4-yl-4-(trifluoromethyl)-N-[[3- | 9 | 456.1 |
| | (trifluoromethyloxy)-phenyl]-methyl]-thiazole-5- | | |
| | carboxylic acid amide | | |
| 42 | N-[1-(4-Chlorophenyl)-ethyl]-2-morpholin-4-yl-4- | 9 | 420.1 |
| | (trifluoromethyl)-thiazole-5-carboxylic acid amide | | |
| 43 | N-[(4-Chlorophenyl)-methyl]-2-morpholin-4-yl-4- | 9 | 406.0 |
| | (trifluoromethyl)-thiazole-5-carboxylic acid amide | | |
| 44 | 2-Morpholin-4-yl-4-(trifluoromethyl)-N-[1-[3- | 9 | 454.1 |
| | (trifluoromethyl)phenyl]-ethyl]-thiazole-5-carboxylic | | |
| | acid amide | | |

### Pharmacological Experiments

### Method I. Fluorescence Assay Using a Voltage Sensitive Dye (Fluorimetry)

Human CHO-K1 cells expressing KCNQ2/3 channels are cultivated adherently at 37° C., 5% CO₂ and 95% humidity in cell culture bottles (e.g. 80 cm² TC flasks, Nunc) with DMEM-high glucose (Sigma Aldrich, D7777) including 10% FCS (PAN Biotech, e.g. 3302-P270521) or alternatively MEM Alpha Medium (1×, liquid, Invitrogen, #22571), 10% fetal calf serum (FCS) (Invitrogen, #10270-106, heat-inactivated) and the necessary selection antibiotics.

Before being sown out for the measurements, the cells are washed with 1×DPBS buffer Ca²⁺/Mg²⁺-free (e.g. Invitrogen, #14190-094) and detached from the bottom of the culture vessel by using Accutase (PAA Laboratories, #L11-007) (incubation with Accutase for 15 min at 37° C.). The cell number is determined using a CASY™ cell counter (TCC, Scharfe System). Depending on the optimal density for each individual cell line, 20,000-30,000 cells/well/100 μl are seeded onto 96-well Corning™ CeIIBIND™ assay plates (Flat Clear Bottom Black Polystyrene Microplates, #3340). Freshly seeded cells are then left to settle for one hour at room temperature, followed by incubation for 24 hours at 37° C., 5% CO₂ and 95% humidity.

The voltage-sensitive fluorescent dye from the Membrane Potential Assay Kit (Red™ Bulk format part R8123 for FLIPR, MDS Analytical Technologies™) is prepared by dissolving the contents of one vessel Membrane Potential Assay Kit Red Component A in 200 ml of extracellular buffer (ES buffer, 120 mM NaCl, 1 mM KCl, 10 mM HEPES, 2 mM CaCl₂, 2 mM MgCl₂, 10 mM glucose; pH 7.4). After removal of the nutrient medium, the cells are washed once with 200 μl of ES buffer, then loaded for 45 min at room temperature in 100 μl of dye solution in the dark.

Fluorescence measurements are carried out in a BMG Labtech FLUOstar™, BMG Labtech NOVOstar™ or BMG Labtech POLARstar™ instrument (525 nm excitation, 560 nm emission, Bottom Read mode). After incubation with the dye, 50 μl of the test substances in the desired concentrations, or 50 μl of ES buffer for control purposes, are applied to the wells of the assay plate and incubated for 30 min at room temperature while being shielded from light. The fluorescence intensity of the dye is then measured for 5 min and the fluorescence value F₁ of each well is thus determined at a given, constant time. 15 μl of a KCl solution are then added to each well (final concentration of potassium ions 92 mM). The change in fluorescence intensity is subsequently monitored until all the relevant values have been obtained (mainly 5-30 min). At a given time post KCl application, a fluorescence value F₂ is determined, in this case at the time of the fluorescence peak.

For calculation, the fluorescence intensity F₂ is corrected for the fluorescence intensity F₁, and the activity (ΔF/F) of the target compound on the potassium channel is determined as follows:

$$( \frac{{F}_{2} - {F}_{1}}{F - 1} ) \times 100 = \frac{Δ F}{F} ( \% )$$

In order to determine whether a substance has agonistic activity,

$$\frac{Δ F}{F}$$

can be related to

$${( \frac{Δ F}{F} )}_{K}$$

of control wells.

$${( \frac{Δ F}{F} )}_{K}$$

is determined by adding to the well only the buffer solution instead of the test substance, determining the value F_{1K} of the fluorescence intensity, adding the potassium ions as described above, and measuring a value F_{2K} of the fluorescence intensity. F_{2K} and F_{1K} are then calculated as follows:

$$( \frac{{F}_{2 K} - {F}_{1 K}}{{F}_{1 K}} ) \times 100 = {( \frac{Δ F}{F} )}_{K} ( \% )$$

A substance has an agonistic activity on the potassium channel if

$$\frac{Δ F}{F}$$

is greater than

$${( \frac{Δ F}{F} )}_{K} : \frac{Δ F}{F} > {( \frac{Δ F}{F} )}_{K}$$

Independently of the comparison of

$$\frac{Δ F}{F}$$

with

$${( \frac{Δ F}{F} )}_{K}$$

it is possible to conclude that a target compound has agonistic activity if

$$\frac{Δ F}{F}$$

increases dose dependently.

Calculations of EC₅₀ and IC₅₀ values are carried out with the aid of ‘Prism v4.0’ software (GraphPad Software™).

### Method II. Low-Intensity Tail Flick Test (Rat)

In the low-intensity tail flick test, the determination of the antinociceptive effect of the compounds according to the invention towards an acute noxious thermal stimulus is carried out by measuring the withdrawal reflex of the rat tail (tail flick) in response to a radiant heat beam (analgesia meter; model 2011 of the company Rhema Labortechnik, Hofheim, Germany) according to the method described by D'Amour and Smith (J. Pharm. Exp. Ther. 72, 74 79 (1941). To this end, the rats were placed in a plexiglas restrainer, and a low-intensity radiant heat beam (48° C.) was focused onto the dorsal surface of the tail root. The stimulus intensity was adjusted to result in a mean pre-drug control withdrawal latency of about 7 s, thus also allowing a supraspinal modulation of the spinally mediated acute nociceptive reflex. A cutoff time of 30 s was applied to avoid tissue damage. Male Sprague-Dawley rats (Janvier, Le Genest St. Isle, Frankreich) with weights of 200-250 g were used. 10 rats were used per group. Before administration of a compound according to the invention, the animals were pre-tested twice in the course of five minutes and the mean of these measurements was calculated as the pre-test mean. The antinociceptive effect was determined at 20, 40 and 60 min after peroral compound administration. The antinociceptive effect was calculated based on the increase in the tail withdrawal latency according to the following formula and is expressed as percentage of the maximum possible effect (MPE [%]):

MPE=[(T₁−T₀)/(T₂−T₀)]*100

In this, T₀ is the control latency time before and T₁ the latency time after administration of the compound, T₂ is the cutoff time and MPE is the maximum possible effect. Employing variant analysis (repeated measures ANOVA) allowed testing of statistically significant differences between the compounds according to the invention and the vehicle group. The significance level was set to p≦0.05.

### Pharmacological Data

The pharmacological effects of the compounds according to the invention were determined as described hereinbefore (pharmacological experiments, methods I and II respectively).

The corresponding pharmacological data are summarized in Table 2.

TABLE 2

| | Fluorimetry | Fluorimetry | Low intensity tail flick, |
|---|---|---|---|
| | % efficacy | EC₅₀/IC₅₀ | rat, peroral, MPE (dose) |
| Example | (retigabine = 100%) | [nM] | [mg/kg] |
| 1 | 137 | 136 | |
| 2 | 147 | 315 | |
| 3 | 171 | 564 | |
| 4 | 187 | 263 | 42 (10) |
| 5 | 218 | 1347 | |
| 7 | 109 | 482 | |
| 9 | 104 | 379 | |
| 10 | 165 | 140 | |
| 12 | 145 | 1466 | |
| 13 | 241 | 319 | |
| 14 | 176 | 194 | |
| 15 | 247 | 82 | |
| 16 | 178 | 1417 | |
| 17 | 180 | 499 | |
| 20 | 88 | 217 | |
| 21 | 132 | 1045 | |
| 22 | 78 | 459 | |
| 23 | 98 | 1138 | |
| 25 | 48 | 1260 | |
| 35 | 131 | 1265 | |
| 36 | 125 | 1104 | |
| 38 | 102 | 1255 | |
| 40 | 78 | 1284 | |

1. A compound of general formula (I) 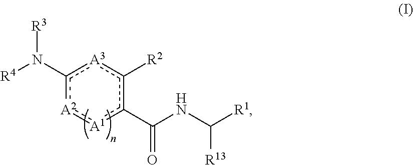
wherein
A¹ represents CR⁵ or N;
A² represents CR⁶, N, O, S or NR⁷;
A³ represents CR⁸ or N, and
n denotes 0 or 1,
on the condition, that
if n denotes 0, then A² represents O, S or NR⁷, or
if n denotes 1, then A² represents CR⁶ or N,
wherein
R⁵ is selected from F, Cl, Br, CN, CH₃, CF₃, CHF₂, CH₂F, OCH₃, C₂H₅, SCH₃, OCF₃, OCHF₂ or OCH₂F;
R⁶ is selected from H, F, Cl, Br, CN, CH₃, CF₃, CHF₂, CH₂F, OCH₃, C₂H₅, SCH₃, OCF₃, OCHF₂ or OCH₂F;
R⁷ represents C₁₋₄-aliphatic residue or C₃₋₅-cycloaliphatic residue, in each case unsubstituted or mono- or polysubstituted;
R⁸ is selected from H, F, Cl, Br, CN, CH₃, CF₃, CHF₂, CH₂F, OCH₃, C₂H₅, SCH₃, OCF₃, OCHF₂ or OCH₂F;
with the proviso, that,
if n denotes 1, then at least one of A¹, A² and A³ denotes N,
with the proviso, that
if n denotes 1 and A³ denotes N, then A¹ and/or A² denotes N,
and with the proviso, that
if n denotes 1 and A² denotes N and A¹ denotes CR⁵ and A³ denotes CR⁸, then R⁵ denotes F, Cl, CH₃, CF₃, CHF₂ or CH₂F;
R¹³ represents H or C₁₋₄-aliphatic residue,
R¹ represents C₁₋₁₀-aliphatic residue, unsubstituted or mono- or polysubstituted; or
C₃₋₁₀-cycloaliphatic residue or 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted;
or
aryl or heteroaryl, in each case unsubstituted or mono- or polysubstituted and in each case optionally linked via a C₁₋₄-aliphatic group, which in turn may be unsubstituted or mono- or polysubstituted;
R² represents a C₁₋₆-aliphatic residue, unsubstituted or mono- or polysubstituted; a C₃₋₆-cycloaliphatic residue or a 3 to 7 membered heterocycloaliphatic residue, in each case unsubstituted or mono- or polysubstituted,
or
denotes S—R⁹, O—R¹⁰ or N(R¹¹R¹²),
wherein
R⁹ and R¹⁰ in each case represent C₁₋₆-aliphatic residue, unsubstituted or mono- or polysubstituted; C₃₋₆-cycloaliphatic residue or 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted;
with the proviso, that if R⁹ or R¹⁰ denote a 3 to 7 membered heterocycloaliphatic residue, than the 3 to 7 membered heterocycloaliphatic residue is linked via a carbon atom,
R¹¹ represents C₁₋₆-aliphatic residue, unsubstituted or mono- or polysubstituted; C₃₋₆-cycloaliphatic residue or a 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted;
with the proviso that if R¹¹ denotes a 3 to 7 membered heterocycloaliphatic residue, the 3 to 7 membered heterocycloaliphatic residue is linked via a carbon atom; and
R¹² denotes C₁₋₆-aliphatic residue, unsubstituted or mono- or polysubstituted;
or
R¹¹ and R¹² form together with the nitrogen atom connecting them a 3 to 7 membered heterocycloaliphatic residue, unsubstituted or mono- or polysubstituted;
R³ represents C₁₋₁₀-aliphatic residue, unsubstituted or mono- or polysubstituted; or
C₃₋₁₀-cycloaliphatic residue or 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted;
or
aryl or heteroaryl, in each case unsubstituted or mono- or polysubstituted and in each case optionally linked via a C₁₋₄-aliphatic group, which in turn may be unsubstituted or mono- or polysubstituted;
on the condition that if R³ denotes a 3 to 10 membered heterocycloaliphatic residue or a heteroaryl, the 3 to 10 membered heterocycloaliphatic residue or the heteroaryl is linked via a carbon atom;
and
R⁴ denotes H or C₁₋₁₀-aliphatic residue, unsubstituted or mono- or polysubstituted;
or
R³ and R⁴ form together with the nitrogen atom connecting them a 3 to 10 membered heterocycloaliphatic residue, unsubstituted or mono- or polysubstituted;
in which an “aliphatic group” and “aliphatic residue” may in each case be branched or unbranched, saturated or unsaturated,
in which a “cycloaliphatic residue” and a “heterocycloaliphatic residue” may in each case be saturated or unsaturated,
in which “mono- or polysubstituted” with respect to an “aliphatic group”, an “aliphatic residue”, a “cycloaliphatic residue” and a “heterocycloaliphatic residue” relates, with respect to the corresponding residues or groups, to the substitution of one or more hydrogen atoms each independently of one another by at least one substituent selected from the group consisting of F, Cl, Br, I, NO₂, NH₂, NH(C₁₋₄-aliphatic residue), N(C₁₋₄-aliphatic residue)₂, NH—C(═O)—C₁₋₄-aliphatic residue, N(C₁₋₄ aliphatic residue)-C(═O)—C₁₋₄ aliphatic residue, NH—S(═O)₂—C₁₋₄ aliphatic residue, N(C₁₋₄ aliphatic residue)-S(═O)₂—C₁₋₄ aliphatic residue, ═O, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂—C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, CHO, COOH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, C(═O)NH₂, a C(═O)—NH(C₁₋₄-aliphatic residue) and C(═O)—N(C₁₋₄-aliphatic residue)₂;
in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂, 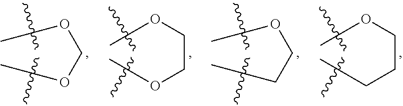 [SMILES: *OCO* ; *OCCO* ; *CCO* ; *CCCO*]
NH(C₁₋₄-aliphatic residue), N(C₁₋₄-aliphatic residue)₂, NH—C(═O)—C₁₋₄-aliphatic residue, N(C₁₋₄ aliphatic residue)-O(═O)—C₁₋₄ aliphatic residue, NH—S(═O)₂—C₁₋₄ aliphatic residue, N(C₁₋₄ aliphatic residue)-S(═O)₂—C₁₋₄ aliphatic residue, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂—C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, C(═O)H, C(═O)OH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, C(═O)—NH(C₁₋₄-aliphatic residue) and C(═O)—N(C₁₋₄-aliphatic residue)₂;
in the form of an individual single stereoisomer or a mixture of the stereoisomers in any mixing ratio, and/or in the form of a free compound, a solvate and/or a physiologically acceptable salt.

2. The compound according to claim 1, wherein
A¹ represents CR⁵ or N;
A² represents CR⁶, N, O, S or NR⁷;
A³ represents CR⁸ or N, and
n denotes 0 or 1,
on the condition, that
if n denotes 0, then A² represents O, S or NR⁷, or
if n denotes 1, then A² represents CR⁶ or N,
wherein
R⁵ is selected from F, Cl, Br, CN, CH₃, CF₃, CHF₂, CH₂F, OCH₃, C₂H₅, SCH₃, OCF₃, OCHF₂ or OCH₂F;
R⁶ is selected from H, F, Cl, Br, CN, CH₃, CF₃, CHF₂, CH₂F, OCH₃, C₂H₅, SCH₃, OCF₃, OCHF₂ or OCH₂F;
R⁷ represents C₁₋₄-aliphatic residue or C₃₋₅-cycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
R⁸ is selected from H, F, Cl, Br, CN, CH₃, CF₃, CHF₂, CH₂F, OCH₃, C₂H₅, SCH₃, OCF₃, OCHF₂ or OCH₂F;
with the proviso, that,
if n denotes 1, then at least one of A¹, A² and A³ denotes N,
with the proviso, that
if n denotes 1 and A³ denotes N, then A¹ and/or A² denotes N,
and with the proviso, that
if n denotes 1 and A² denotes N and A¹ denotes CR⁵ and A³ denotes CR⁸, then R⁵ denotes F, Cl, CH₃, CF₃, CHF₂ or CH₂F;
R¹³ represents H or C₁₋₄-aliphatic residue,
R¹ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH,
and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄ aliphatic residue, OCF₃, SH, SCF₃, S—C₁₋₄-aliphatic residue, CF₃, CN, C₁₋₄-aliphatic residue and C(═O)OH,
or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, 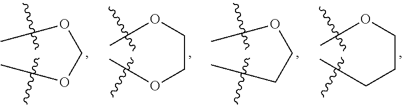 [SMILES: *OCO* ; *OCCO* ; *CCO* ; *CCCO*]
benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, a C₁₋₄-aliphatic residue, C(═O)OH, C(═O)CH₃, C(═O)C₂H₅, C(═O)—OCH₃ and C(═O)—OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, an NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O)OH,
R² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or
denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
or
denotes S—R⁹, O—R¹⁰ or N(R¹¹R¹²);
wherein
R⁹ and R¹⁰ in each case represent C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or in each case represent C₃₋₆-cycloaliphatic residue or 3 to 7 membered heterocyclo-aliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
on the condition that if R⁹ or R¹⁰ denote a 3 to 7 membered heterocycloaliphatic residue, the 3 to 7 membered heterocycloaliphatic residue is linked via a carbon atom, R¹¹ denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue, and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C(═O)—O—C₁₋₄-aliphatic residue, C₃₋₆-cycloaliphatic residue, and a 3 to 7 membered heterocycloaliphatic residue,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH, and
wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O)OH,
on the condition that if R¹¹ denotes a 3 to 7 membered heterocycloaliphatic residue, the 3 to 7 membered heterocycloaliphatic residue is linked via a carbon atom,
and
R¹² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄aliphatic residue,
or
R¹¹ and R¹² form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₆-cycloaliphatic residue and 3 to 7 membered heterocycloaliphatic residue,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
wherein the 3 to 10 membered heterocycloaliphatic residue formed by R¹¹ and R¹² together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)—OH, C(═O)—CH₃, C(═O)—C₂H₅, C(═O)—OCH₃ and C(═O)—OC₂H₅, C₃₋₆-cycloaliphatic residue, 3 to 7 membered heterocycloaliphatic residue, 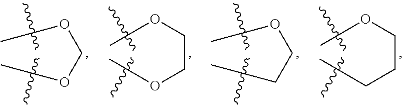 [SMILES: *OCO* ; *OCCO* ; *CCO* ; *CCCO*]
benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
R³ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C(═O)—O—C₁₋₄-aliphatic residue, C₃₋₆-cycloaliphatic residue and 3 to 7 membered heterocycloaliphatic residue,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O)OH,
on the condition that if R³ denotes a 3 to 10 membered heterocycloaliphatic residue, the 3 to 10 membered heterocycloaliphatic residue is linked via a carbon atom,
or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, 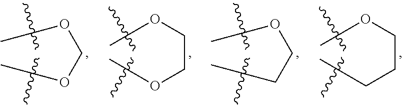 [SMILES: *OCO* ; *OCCO* ; *CCO* ; *CCCO*]
benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O)OH,
R⁴ denotes H or C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue,
or
R³ and R⁴ form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)OH, C₃₋₆-cycloaliphatic residue and 3 to 7 membered heterocycloaliphatic residue,
wherein the C₁₋₄-aliphatic residue in each case may be unsubstituted or mono- or polysubstituted with at least one substituent selected from the group consisting of F, Cl, Br, I, ═O, OH, OCF₃, CF₃ and unsubstituted O—C₁₋₄-aliphatic residue, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and
wherein the 3 to 10 membered heterocycloaliphatic residue formed by R³ and R⁴ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, 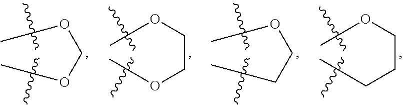 [SMILES: *OCO* ; *OCCO* ; *CCO* ; *CCCO*]
benzyl, phenyl, thienyl, pyridyl, furyl, thiazolyl and oxazolyl,
and wherein the 3 to 10 membered heterocycloaliphatic residue formed by R³ and R⁴ together with the nitrogen atom connecting them may optionally be condensed with a C₃₋₁₀-cycloaliphatic residue or a 3 to 10 membered heterocycloaliphatic residue,
wherein the C₃₋₁₀-cycloaliphatic residue or the 3 to 10 membered heterocycloaliphatic residue condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, ═O, OH, O—C₁₋₄-aliphatic residue, OCF₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl, pyridyl, furyl, thiazolyl and oxazolyl,
wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and
wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O)OH, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH.

3. The compound according to claim 1, wherein
n denotes 0 and the compound is represented by general formula (Ib), 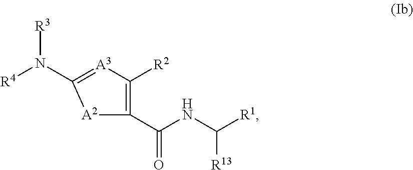
wherein
A² represents O and A³ represents CR⁸; or
A² represents S and A³ represents CR⁸; or
A² represents NR⁷ and A³ represents CR⁸; or
A² represents O and A³ represents N; or
A² represents S and A³ represents N; or
A² represents NR⁷ and A³ represents N.

4. The compound according to claim 1, wherein
n denotes 1 and the compound is represented by general formula (Ia), 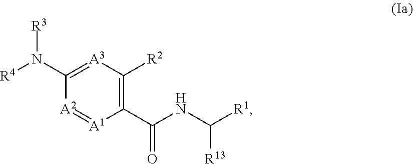
wherein
A¹ represents N and A² represents CR⁶ and A³ represents CR⁸; or
A¹ represents CR⁵ and A² represents N and A³ represents CR⁸; or
A¹ represents N and A² represents N and A³ represents CR⁸; or
A¹ represents N and A² represents CR⁶ and A³ represents N; or
A¹ represents CR⁵ and A² represents N and A³ represents N; or
A¹ represents N and A² represents N and A³ represents N.

5. The compound according to claim 1, wherein
R⁵ denotes F, Cl, CH₃, OCH₃ or CH₂CH₃; and/or
R⁶ denotes H; and/or
R⁷ denotes CH₃, CH₂CH₃ or cyclopropyl; and/or
R⁸ denotes H.

6. The compound according to claim 1, wherein
R¹ represents the partial structure (T1), 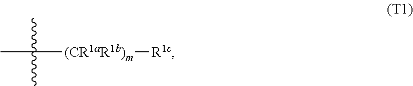
wherein
m denotes 0, 1, or 2,
R^{1a} and R^{1b} each independently of one another represent H, F, Cl, Br, I, O—C₁₋₄-aliphatic residue or C₁₋₄-aliphatic residue,
R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue,
wherein the C₁₋₄-aliphatic residue in each case may be unsubstituted or mono- or polysubstituted with at least one substituent selected from the group consisting of F, Cl, CF₃ and O—C₁₋₄-aliphatic residue,
or
denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue,
or
denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
wherein benzyl, phenyl, thienyl and pyridyl may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃C₁₋₄-aliphatic residue and C(═O)OH.

7. The compound according to claim 6, wherein
m denotes 1 or 2,
R^{1a} and R^{1b} represent H,
R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue,
or
denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
or
m denotes 0 and
R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃C₁₋₄-aliphatic residue and C(═O)OH.

8. The compound according to claim 1, wherein
R² is selected from the group consisting of
CH₃, C₂H₅, CH₂CH₂CH₃, CH(CH₃)₂, CH₂CH₂CH₂CH₃, CH₂CH(CH₃)₂, CH(CH₃)CH₂CH₃, C(CH₃)₃, CH₂-cyclopropyl, OCH₃, OC₂H₅, OCH₂CH₂CH₃, OCH(CH₃)₂, O-cyclopropyl, SCH₃, SC₂H₅, SCH₂CH₂CH₃, SCH(CH₃)₂, S-cyclopropyl, cyclopropyl, cyclobutyl, cyclopentyl, cyclohexyl, N(CH₃)₂, N(CH₃)C₂H₅, N(CH₃)CH₂CH₂CH₃, N(CH₃)CH(CH₃)₂, N(CH₃)-cyclopropyl, N(C₂H₅)₂, N(C₂H₅)CH₂CH₂CH₃, N(C₂H₅)CH(CH₃)₂, N(C₂H₅)-cyclopropyl, N-aziridinyl, N-azetidinyl, N-pyrrolidinyl, N-piperidinyl or N-morpholinyl,
in each case unsubstituted or mono- or polysubstituted with F, OH and/or OCH₃.

9. The compound according to claim 1, wherein
R³ represents the partial structure (T2), 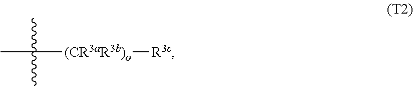
wherein
o denotes 0, 1, 2 or 3,
R^{3a} and R^{3b} each independently of one another represent H, F, Cl, Br, I, O—C₁₋₄-aliphatic residue or C₁₋₄-aliphatic residue or together denote ═O, and
R^{3c} denotes C₁₋₄-aliphatic residue, unsubstituted or mono- or polysubstituted with at least one substituent selected from the group consisting of F, Cl, Br, I, ═O, O—C₁₋₄-aliphatic residue, CF₃ and C₁₋₄-aliphatic residue,
wherein the C₁₋₄-aliphatic residue in each case may be unsubstituted or mono- or polysubstituted with at least one substituent selected from the group consisting of F, Cl, CF₃ and O—C₁₋₄-aliphatic residue,
or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue,
or denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
wherein benzyl, phenyl, thienyl and pyridyl, may in each case may be unsubstituted or mono- or polysubstituted, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃, C₁₋₄-aliphatic residue and C(═O)—OH,
and
R⁴ denotes H or unsubstituted C₁₋₄-aliphatic residue or C₁₋₄-aliphatic residue, monosubstituted with OCH₃,
or
R³ and R⁴ form together with the nitrogen atom connecting them a 3 to 10 membered heterocycloaliphatic residue, unsubstituted or mono- or polysubstituted with at least one substituent selected from the group consisting of F, Cl, Br, I, OH, ═O, C(═O)OH, O—C₁₋₄-aliphatic residue, OCF₃, SCF₃, S—C₁₋₄-aliphatic residue, CF₃, C₁₋₄-aliphatic residue, cyclopropyl, cyclobutyl and cyclopentyl,
wherein the C₁₋₄-aliphatic residue is in each case unsubstituted or mono- or polysubstituted with at least one substituent selected from the group consisting of F, Cl, OH, ═O, CF₃ and O—C₁₋₄-aliphatic residue,
and wherein the 3 to 10 membered heterocycloaliphatic residue formed by R³ and R⁴ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, O—C₁₋₄-aliphatic residue, OCF₃, SCF₃, S—C₁₋₄-aliphatic residue, CF₃, C₁₋₄-aliphatic residue, C(═O)OH and C₃₋₆-cycloaliphatic residue,
wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and
wherein the C₃₋₆-cycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, SCF₃, S—C₁₋₄-aliphatic residue, CF₃, C₁₋₄-aliphatic residue and C(═O)OH,
and wherein the 3 to 10 membered heterocycloaliphatic residue formed by R³ and R⁴ together with the nitrogen atom connecting them may optionally be condensed with a C₃₋₆-cycloaliphatic residue, or a 3 to 7 membered heterocycloaliphatic residue, wherein the C₃₋₆-cycloaliphatic residue or the 3 to 7 membered heterocycloaliphatic residue condensed in this way can for their part be respectively unsubstituted or mono- or polysubstituted with at least one substituent selected from the group consisting of F, Cl, Br, I, ═O, OH, O—C₁₋₄-aliphatic residue, OCF₃, SCF₃, CF₃, CN, C₁₋₄-aliphatic residue, C(═O)OH, C(═O)CH₃, C(═O)C₂H₆, C(═O)OCH₃ and C(═O)OC₂H₆.

10. The compound according to claim 1, wherein
R³ represents the partial structure (T2) 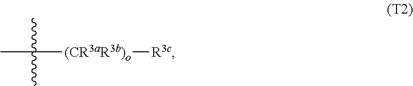
wherein
o denotes 0, 1, 2 or 3,
R^{3a} and R^{3b} each independently of one another represent H, F, CH₃ or OCH₃, or together denote ═O,
R^{3c} denotes C₁₋₄-aliphatic residue, unsubstituted or mono- or polysubstituted with at least one substituent selected from the group consisting of F, Cl, Br, I, ═O, O—C₁₋₄-aliphatic residue, and CF₃,
or denotes cyclopropyl, cyclobutyl, cyclopentyl or cyclohexyl, pyrrolidinyl, morpholinyl, piperazinyl, piperidinyl or tetrahydropyranyl, in each case unsubstituted or mono- or polysubstituted with at least one substituent selected from the group consisting of F, Cl, Br, I, O—C₁₋₄-aliphatic residue, CF₃ and C₁₋₄-aliphatic residue,
or denotes aryl or heteroaryl, in each case unsubstituted or mono- or polysubstituted with at least one substituent selected from the group consisting of F, Cl, Br, OH, O—C₁₋₄-aliphatic residue, OCF₃, CF₃, CN, and C₁₋₄-aliphatic residue,
and
R⁴ denotes H, CH₃, CH₂CH₃, CH₂CH₂OCH₃ or CH₂CH₂CH₂OCH₃,
or
R³ and R⁴ form together with the nitrogen atom connecting them a heteroaliphatic residue, selected from the group consisting of morpholinyl, piperidinyl, pyrrolidinyl, azetidinyl, oxazepanyl, tetrahydroquinolinyl, tetrahydroisoquinolinyl, thiomorpholinyl, azepanyl, tetrahydroimidazo[1,2-a]pyrazinyl, octahydropyrrolo[1,2-a]pyrazinyl, 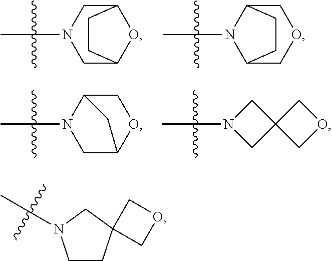
dihydroindolinyl, or dihydroisoindolyl, in each case unsubstituted or mono- or polysubstituted with at least one substituent selected from the group consisting of F, Cl, OH, ═O, C(═O)OH, OCH₃, OCH₂CH₃, OCF₃, SCF₃, CF₃, C(═O)CH₃, C(═O)OCH₃, CH₂CF₃, CH₂OH, CH₂OCH₃, CH₂CH₂OCH₃, CH₃, CH₂CH₃, CH₂CH₂CH₃, CH(CH₃)₂ and cyclopropyl.

11. The compound according to claim 1, wherein
A¹ represents CR⁵, N;
A² represents CR⁶, N, O, S or NR⁷;
A³ represents CR⁸ or N, and
n denotes 0 or 1,
on the condition, that
if n denotes 0, then A² represents O, S or NR⁷, or
if n denotes 1, then A² represents CR⁶ or N,
wherein
R⁵ denotes F, Cl, CH₃, OCH₃ or CH₂CH₃; and/or
R⁶ denotes H; and/or
R⁷ denotes CH₃, CH₂CH₃ or cyclopropyl; and/or
R⁸ denotes H;
with the proviso, that,
if n denotes 1, then at least one of A¹, A² and A³ denotes N,
with the proviso, that
if n denotes 1 and A³ denotes N, then A¹ and/or A² denotes N,
and with the proviso, that
if n denotes 1 and A² denotes N and A¹ denotes CR⁵ and A³ denotes CR⁸, then R⁵ denotes F, Cl, CH₃, CF₃, CHF₂ or CH₂F;
R¹³ represents H or CH₃;
R¹ represents the partial structure (T1), 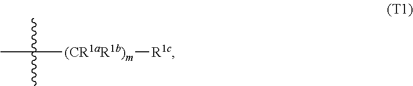
wherein
m denotes 1 or 2,
R^{1a} and R^{1b} represent H and
R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue,
or
denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue,
or wherein
m denotes 0 and
R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)—CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl,
wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, an O—C₁₋₄-aliphatic residue, OCF₃, CF₃C₁₋₄-aliphatic residue and C(═O)OH;
R² is selected from the group consisting of CH₃, C₂H₅, CH₂CH₂CH₃, CH(CH₃)₂, CH₂CH₂CH₂CH₃, CH₂CH(CH₃)₂, CH(CH₃)CH₂CH₃, C(CH₃)₃, CH₂-cyclopropyl, OCH₃, OC₂H₅, OCH₂CH₂CH₃, OCH(CH₃)₂, O-cyclopropyl, SCH₃, SC₂H₅, SCH₂CH₂CH₃, SCH(CH₃)₂, S-cyclopropyl, cyclopropyl, cyclobutyl, cyclopentyl, cyclohexyl, N(CH₃)₂, N(CH₃)C₂H₅, N(CH₃)CH₂CH₂CH₃, N(CH₃)CH(CH₃)₂, N(CH₃)-cyclopropyl, N(C₂H₅)₂, N(C₂H₅)CH₂CH₂CH₃, N(C₂H₅)CH(CH₃)₂, N(C₂H₅)-cyclopropyl, N-aziridinyl, N-azetidinyl, N-pyrrolidinyl, N-piperidinyl or N-morpholinyl,
in each case unsubstituted or mono- or polysubstituted with F, OH and/or OCH₃;
R³ represents the partial structure (T2) 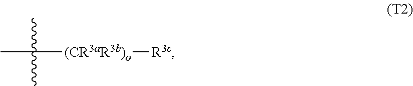
wherein
denotes 0, 1, 2 or 3,
R^{3a} and R^{3b} each independently of one another represent H, F, CH₃ or OCH₃, or together denote ═O,
R^{3c} denotes C₁₋₄-aliphatic residue, unsubstituted or mono- or polysubstituted with at least one substituent selected from the group consisting of F, Cl, Br, I, ═O, O—C₁₋₄-aliphatic residue, and CF₃,
or denotes cyclopropyl, cyclobutyl, cyclopentyl or cyclohexyl, pyrrolidinyl, morpholinyl, piperazinyl, piperidinyl or tetrahydropyranyl,
in each case unsubstituted or mono- or polysubstituted with at least one substituent selected from the group consisting of F, Cl, Br, I, O—C₁₋₄-aliphatic residue, CF₃ and C₁₋₄-aliphatic residue,
or denotes aryl or heteroaryl, in each case unsubstituted or mono- or polysubstituted with at least one substituent selected from the group consisting of F, Cl, Br, OH, O—C₁₋₄-aliphatic residue, OCF₃, CF₃, CN, and C₁₋₄-aliphatic residue,
and
R⁴ denotes H, CH₃, CH₂CH₃, CH₂CH₂OCH₃ or CH₂CH₂CH₂OCH₃,
or
R³ and R⁴ form together with the nitrogen atom connecting them a morpholinyl, piperidinyl, pyrrolidinyl, azetidinyl, oxazepanyl, tetrahydroquinolinyl, tetrahydroisoquinolinyl, thiomorpholinyl, azepanyl, tetrahydroimidazo[1,2-a]pyrazinyl, octahydropyrrolo[1,2-a]-pyrazinyl, 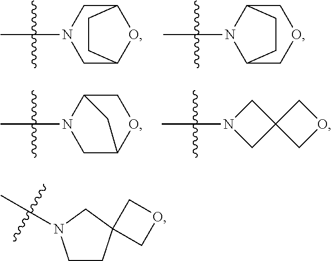
dihydroindolinyl, or dihydroisoindolyl, in each case unsubstituted or mono- or polysubstituted with at least one substituent selected from the group consisting of F, Cl, OH, ═O, C(═O)OH, OCH₃, OCH₂CH₃, OCF₃, SCF₃, CF₃, C(═O)CH₃, C(═O)OCH₃, CH₂CF₃, CH₂OH, CH₂OCH₃, CH₂CH₂OCH₃, CH₃, CH₂CH₃, CH₂CH₂CH₃, CH(CH₃)₂ and cyclopropyl.

12. The compound according to claim 1, wherein the compound is selected from the group consisting of:
1 N-[(4-Chlorophenyl)-methyl]-4-ethylsulfanyl-6-methyl-2-morpholin-4-yl-pyrimidine-5-carboxylic acid amide;
2 N-[(4-Chlorophenyl)-methyl]-4,6-dimethoxy-2-morpholin-4-yl-pyrimidine-5-carboxylic acid amide;
3 N-[(4-Chlorophenyl)-methyl]-4-ethylsulfanyl-2-methyl-6-morpholin-4-yl-pyridine-3-carboxylic acid amide;
4 N-[(4-Chlorophenyl)-methyl]-2-methyl-6-morpholin-4-yl-4-propyl-pyridine-3-carboxylic acid amide;
5 N-(4,4-Dimethyl-pentyl)-4-ethoxy-2-methyl-6-morpholin-4-yl-pyridine-3-carboxylic acid amide;
6 N-(4,4-Dimethyl-pentyl)-4-[(3R)-3-fluoro-pyrrolidin-1-yl]-2-methyl-6-morpholin-4-yl-pyridine-3-carboxylic acid amide;
7 N-[(4-Chlorophenyl)-methyl]-3-isopropyl-5-morpholin-4-yl-pyrazine-2-carboxylic acid amide;
8 N-[(4-Chlorophenyl)-methyl]-3-isopropyl-5-morpholin-4-yl-pyridine-2-carboxylic acid amide;
9 4-Isopropyl-2-(methyl-tetrahydro-pyran-4-yl-amino)-N-[1-[3-(trifluoromethyloxy)-phenyl]-ethyl]-thiazole-5-carboxylic acid amide;
10 4-Ethylsulfanyl-N-[(3-fluorophenyl)-methyl]-6-methyl-2-morpholin-4-yl-pyrimidine-5-carboxylic acid amide;
11 4-Ethylsulfanyl-N-[(3-fluorophenyl)-methyl]-2-methyl-6-morpholin-4-yl-pyridine-3-carboxylic acid amide;
12 4-Ethylsulfanyl-N-[(4-fluorophenyl)-methyl]-2-methyl-6-morpholin-4-yl-pyridine-3-carboxylic acid amide;
13 N-(4,4-Dimethyl-pentyl)-4-ethylsulfanyl-2-methyl-6-morpholin-4-yl-pyridine-3-carboxylic acid amide;
14 N-[(4-Chlorophenyl)-methyl]-4-ethylsulfanyl-2-methyl-6-[(3R)-3-methyl-morpholin-4-yl]-pyridine-3-carboxylic acid amide;
15 N-(4,4-Dimethyl-pentyl)-4-ethylsulfanyl-2-methyl-6-[(3R)-3-methyl-morpholin-4-yl]-pyridine-3-carboxylic acid amide;
16 N-[(4-Chlorophenyl)-methyl]-4-cyclopropyl-2-methyl-6-morpholin-4-yl-pyridine-3-carboxylic acid amide;
17 N-[(4-Chlorophenyl)-methyl]-4-isopropyl-2-methyl-6-morpholin-4-yl-pyridine-3-carboxylic acid amide;
18 N-[(4-Chlorophenyl)-methyl]-4-ethoxy-2-methyl-6-morpholin-4-yl-pyridine-3-carboxylic acid amide;
19 N-[(4-Chlorophenyl)-methyl]-4-dimethylamino-2-methyl-6-morpholin-4-yl-pyridine-3-carboxylic acid amide;
20 N-[(4-Chlorophenyl)-methyl]-3-(1-methyl-propyl)-5-morpholin-4-yl-pyrazine-2-carboxylic acid amide;
21 N-(2-Cyclopentyl-ethyl)-3-(1-methyl-propyl)-5-morpholin-4-yl-pyrazine-2-carboxylic acid amide;
22 4-Isopropyl-2-[methyl-(tetrahydro-pyran-4-yl-methyl)-amino]-N-[1-[3-(trifluoromethyloxy)-phenyl]-ethyl]-thiazole-5-carboxylic acid amide;
23 N-[(4-Chlorophenyl)-methyl]-4-isopropyl-2-(methyl-tetrahydro-pyran-4-yl-amino)-thiazole-5-carboxylic acid amide;
24 N-[(4-Chlorophenyl)-methyl]-4-isopropyl-2-[methyl-(tetrahydro-pyran-4-yl-methyl)-amino]-thiazole-5-carboxylic acid amide;
25 N-[(4-Chlorophenyl)-methyl]-4-isopropyl-2-morpholin-4-yl-thiazole-5-carboxylic acid amide;
26 N-[(4-Chlorophenyl)-methyl]-4-isopropyl-2-piperidin-1-yl-thiazole-5-carboxylic acid amide;
27 N-(4,4-Dimethyl-pentyl)-4-isopropyl-2-piperidin-1-yl-thiazole-5-carboxylic acid amide;
28 N-[(4-Chlorophenyl)-methyl]-4-isopropyl-2-([1,4]oxazepan-4-yl)-thiazole-5-carboxylic acid amide;
29 N-(4,4-Dimethyl-pentyl)-4-isopropyl-2-([1,4]oxazepan-4-yl)-thiazole-5-carboxylic acid amide;
30 N-[(3,4-Difluoro-phenyl)-methyl]-2-morpholin-4-yl-4-propyl-thiazole-5-carboxylic acid amide;
31 N-[(4-Chlorophenyl)-methyl]-4-isopropyl-2-morpholin-4-yl-oxazole-5-carboxylic acid amide;
32 N-(4,4-Dimethyl-pentyl)-4-isopropyl-2-morpholin-4-yl-oxazole-5-carboxylic acid amide;
33 N-(4,4-Dimethyl-pentyl)-5-isopropyl-3-methyl-2-morpholin-4-yl-3H-imidazole-4-carboxylic acid amide;
34 N-[(4-Chlorophenyl)-methyl]-5-isopropyl-3-methyl-2-morpholin-4-yl-3H-imidazole-4-carboxylic acid amide;
35 2-(Methyl-tetrahydro-pyran-4-yl-amino)-4-(trifluoromethyl)-N-[1-[3-(trifluoromethyloxy)-phenyl]-ethyl]-thiazole-5-carboxylic acid amide;
36 2-(Methyl-tetrahydro-pyran-4-yl-amino)-4-(trifluoromethyl)-N-[[3-(trifluoromethyloxy)-phenyl]-methyl]-thiazole-5-carboxylic acid amide;
37 N-[1-(4-Chlorophenyl)-ethyl]-2-(methyl-tetrahydro-pyran-4-yl-amino)-4-(trifluoromethyl)-thiazole-5-carboxylic acid amide;
38 N-[(4-Chlorophenyl)-methyl]-2-(methyl-tetrahydro-pyran-4-yl-amino)-4-(trifluoromethyl)-thiazole-5-carboxylic acid amide;
39 N-[(4-Chlorophenyl)-methyl]-2-[methyl-(tetrahydro-pyran-4-yl-methyl)-amino]-4-(trifluoromethyl)-thiazole-5-carboxylic acid amide;
40 2-Morpholin-4-yl-4-(trifluoromethyl)-N-[[3-(trifluoromethyloxy)-phenyl]-ethyl]-thiazole-5-carboxylic acid amide;
41 2-Morpholin-4-yl-4-(trifluoromethyl)-N-[1-[3-(trifluoromethyloxy)-phenyl]-ethyl]-thiazole-5-carboxylic acid amide;
42 N-[1-(4-Chlorophenyl)-ethyl]-2-morpholin-4-yl-4-(trifluoromethyl)-thiazole-5-carboxylic acid amide;
43 N-[(4-Chlorophenyl)-methyl]-2-morpholin-4-yl-4-(trifluoromethyl)-thiazole-5-carboxylic acid amide;
44 2-Morpholin-4-yl-4-(trifluoromethyl)-N-[1-[3-(trifluoromethyl)phenyl]-ethyl]-thiazole-5-carboxylic acid amide;
in the form of a free compound, a solvate and/or a physiologically acceptable salt.

13. A pharmaceutical composition comprising at least one compound according to claim 1
in the form of an individual single stereoisomer or a mixture of the stereoisomers in any mixing ratio, in the form of a free compound and/or in the form of a solvate and/or a physiologically acceptable salt,
and optionally at least one pharmaceutically acceptable auxiliary and/or optionally at least one further active ingredient.

14. A method for treatment and/or prophylaxis of disorders and/or diseases which are mediated, at least in part, by KCNQ2/3 K⁺ channels in a patient in need thereof, said method comprising administering to said patient an effective amount therefor of a compound according to claim 1.

15. The method according to claim 14, wherein the disorders and/or diseases are selected from the group consisting of pain, acute pain, chronic pain, neuropathic pain, muscular pain, visceral pain, inflammatory pain, epilepsy, urinary incontinence, anxiety, dependency, mania, bipolar disorders, migraine, cognitive diseases and dystonia-associated dyskinesias.
